(19) United States
(12) Patent Application Publication
(10) Pub. No.: US 2007/0010526 A1
(43) Pub. Date: Jan. 11, 2007

(54) Therapeutic isoquinoline compounds

(76) Inventors: Markus Haeberlein, Sodertalje (SE); Christof Angst, Wilmington, DE (US); Daniel Hill, Wilmington, DE (US); Robert Jacobs, Wilmington, DE (US); Gary Moore, Wilmington, DE (US); Edward Pierson, Wilmington, DE (US); Ashokkumar Bhikkappa Shenvi, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/494,424

(22) PCT Filed: Nov. 1, 2002
(86) PCT No.: PCT/SE02/01988

§ 371 Date: Mar. 6, 2006

(30) Foreign Application Priority Data
Nov. 1, 2001 (SE) 0103644-1

(51) Int. Cl. A61K 31/496; A61K 31/4709; C07D 403/02; C07D 401/02

(52) U.S. Cl. 514/253.05; 514/310; 544/363; 546/148

(57) ABSTRACT

Provided herein is a compound of the formula (I), wherein said compounds are useful for the treatment of psychiatric disorders including but not limited to depression, generalized anxiety, eating disorders, dementia, panic disorder, and sleep disorders. The compounds may also be useful in the treatment of gastrointestinal disorders, cardiovascular regulation, motor disorders, endocrine disorders, vasospasm and sexual dysfunction. The compounds are 5HT_{1B} and 5HT_{1D} antagonists.

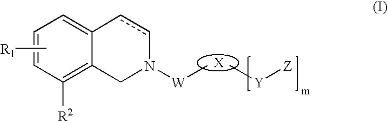

### FIELD OF THE INVENTION

This invention relates to novel isoquinoline derivatives, methods for their preparation, pharmaceutical compositions containing them and their use in therapy.

### BACKGROUND OF THE INVENTION

Serotonin (5-HT) has been implicated in many psychiatric disorders including but not limited to depression, generalized anxiety, eating disorders, dementia, panic disorder, and sleep disorders. Furthermore serotonin has been implicated in gastrointestinal disorders, cardiovascular regulation, motor disorders, endocrine disorders, vasospasm and sexual dysfunction. Serotonin receptors have been subdivided into at least 14 subtypes, see Barnes and Sharp, Neuropharmacology, 1999, 38, 1083-1152, incorporated herein by reference. These various subtypes are responsible for serotonin's action in many pathophysicogical conditions. The 5-HT₁ family of receptors has high affinity for serotonin and comprises five related receptors. This family includes the 5-HT_{1B} and 5-HT_{1D} receptor subtypes. Compounds that interact with the 5-HT₁ family are known to have therapeutic potential in the above mentioned disorders and diseases. In particular, compounds that are 5HT_{1B} and 5HT_{1D} antagonist have been known to be fast acting antidepressants. Compounds that are 5HT_{1B} and 5HT_{1D} agonists have been used in the treatment of migraine.

### SUMMARY OF THE INVENTION

Provided herein is a compound having the formula (I): 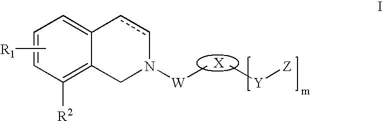
wherein
X is aryl, substituted aryl, heterocyclic or substituted heterocyclic;
W is —(C═O)—, —C(═O)NR^{a}—, —NR^{a}C(═O)—, —C(═O)(CH₂)ₙNR⁸C(═O)—, —C(═S)NR^{a}—, —C(═O)CH₂O—, —SO₂NRⁿ—, —NR^{a}SO₂—, —CH₂NR^{a}—, —C(═O)CH₂—, —CH₂C(═O)— or 5-membered heterocyclic;
R^{a} is —H, alkyl or substituted alkyl;
n is an integer selected from 0, 1, 2, 3 and 4;
Y is —CH₂—, —O—, —S—, —S(═O)—, —C(═O)—, —SO₂—, —N(R)—, —N(R^{b})SO₂—, —SO₂NR^{b}— or a single bond;
Z is —R^{b}, aryl, substituted aryl, heterocyclic, substituted heterocyclic, aryl(C₁-C₄)alkyl, substituted aryl(C₁-C₄)alkyl, —C(═O)OR^{a}, —C(═O)NR^{a}₂, —NHR^{b}, (R^{a})₂N(C₁-C₆)alkyl or —SO₂R^{c};
R^{b} is —H, alkyl, alkanoyl, (C₁-C₆)alkylsulfanyl, aryl, aryl(C₁-C₄)alkyl or aryl(C₁-C₃)alkoxy(C₁-C₄)alkyl;
R^{c} is alkyl, aryl or heterocyclic;
m is an integer selected from 0 and 1;
R¹ is alkyl, halogen, —OR^{a}, —SOₚR^{a}, —NR^{a}₂ or —CN;
p is an integer selected from 0, 1 and 2;
R² is aryl, heterocyclic or a carboxamide wherein the two substituents of the carboxamide nitrogen form a heterocycle containing said amide nitrogen; [symbol]
indicates that the bond represented includes single bonds and double bonds.
Particular compounds of the present invention are those in accord with structural diagram I wherein; R² is represented by the formula II; 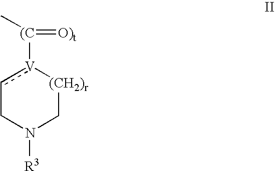
wherein V is N or C;
t is an integer selected from 0 and 1;
r is an integer selected from 1, 2 and 3; — [symbol]
indicates that the bond represented includes single bonds and double bonds; and
R³ is —H, alkyl, substituted alkyl, cycloalkyl, substituted cycloalkyl, aryl(C₁-C₄)alkyl or substituted aryl(C₁-C₄)alkyl.

The term “hydrocarbyl” refers to any structure comprising only carbon and hydrogen atoms up to 14 carbon atoms.

The term “alkyl” used alone or as a suffix or prefix, refers to straight or branched chain hydrocarbyl radicals comprising 1 to about 12 carbon atoms.

The term “alkenyl” refers to straight or branched chain hydrocarbyl radicals having at least one carbon-carbon double bond and comprising at least 2 up to about 12 carbon atoms.

The term “alkynyl” refers to straight or branched chain hydrocarbyl radicals having at least one carbon-carbon triple bond and comprising at least 2 up to about 12 carbon atoms.

The term “cycloalkyl” refers to ring-containing hydrocarbyl radicals comprising at least 3 up to about 12 carbon atoms.

The term “cycloalkenyl” refers to ring-containing hydrocarbyl radicals having at least one carbon-carbon double bond and comprising at least 3 up to about 12 carbon atoms.

The term “cycloalkynyl” refers to ring-containing hydrocarbyl radicals having at least one carbon-carbon triple bond and comprising about 7 up to about 12 carbon atoms.

The term “aromatic” refers to hydrocarbyl radicals having one or more polyunsaturated carbon rings having aromatic character, (e.g., 4n+2 delocalized electrons) and comprising 6 up to about 14 carbon atoms.

The term “aryl” refers to aromatic radicals including both monocyclic aromatic radicals comprising 6 carbon atoms and polycyclic aromatic radicals comprising up to about 14 carbon atoms.

The term “alkylene” refers to divalent alkyl moieties, wherein said moiety serves to link two structures together.

The term “heterocycle” or “heterocyclic” or “heterocyclic moiety” refers to ring-containing monovalent and divalent radicals having one or more heteroatoms, independently selected from N, O and S, as part of the ring structure and comprising at least 3 and up to about 20 atoms in the rings. Heterocyclic moieties may be saturated or unsaturated, containing one or more double bonds, and heterocyclic moieties may contain more than one ring.

The term “heteroaryl” refers to heterocyclic monovalent and divalent radicals having aromatic character.

Heterocyclic moieties include for example monocyclic moieties such as: aziridine, oxirane, thiirane, azetidine, oxetane, thietane, pyrrolidine, pyrroline, imidazolidine, pyrazolidine, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide. In addition heterocyclic moieties include heteroaryl rings such as: pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl. Additionally, heterocyclic moieties encompass polycyclic moieties such as: indole, indoline, quinoline, tetrahydroquinoline, isoquinoline, tetrahydroisoquinoline, 1,4-benzodioxan, coumarin, dihydrocoumarin, benzofuran, 2,3-dihydrobenzofuran, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocyclic moieties include polycyclic heterocyclic moieties wherein the ring fusion between two or more rings comprises more than one bond common to both rings and more than two atoms common to both rings. Examples of such bridged heterocycles include quinuclidine, diazabicyclo[2.2.1]heptane and 7-oxabicyclo[2.2.1]heptane.

The term “halo” or “halogen” refers to fluorine, chlorine, bromine and iodine radicals.

The term “alkoxy” refers to radicals of the general formula —O—R, wherein R is selected from a hydrocarbyl radical. Alkoxy moieties include methoxy, ethoxy, propoxy, isopropoxy, butoxy, t-butoxy, isobutoxy, cyclopropylmethoxy, allyloxy, and propargyloxy.

The term amine or amino refers to radicals of the general formula —NRR′, wherein R and R′ are independently selected from hydrogen or a hydrocarby radical.

### DETAILED DESCRIPTION OF THE INVENTION

Therefore, in a further aspect of the invention, alkyl, alkenyl, alkynyl, cycloalkyl, aryl and heterocyclyl components of X, Z, R¹, R², R³, R^{b} and R^{c} may optionally be substituted with halogen, perhalo(C₁-C₆)alkyl such as trifluoromethyl, mercapto, hydroxy, carboxy, (C₁-C₆)alkoxy, (C₁-C₆) alkylthio, (C₁-C₆)alkylsulfone, (C₁-C₆)alkylsulfoxide, (C₁-C₆)alkoxycarbonyl, (C₁-C₆)alkanoyloxy, (C₁-C₆)alkanoyl, sulfamoyl, carboxamido, mono- or di-(C₁-C₆)alkyl carboxamido, (C₁-C₆)alkanoyl, carbamoyl, —N(C₁-C₆)carbamoyl, —N(C₁-C₆)₂carbamoyl, aryl, heterocyclic, (C₂-C₆)alkenyloxy, (C₂-C₆)alkynyloxy, (C₁-C₆)alkoxy(C₁-C₆)alkoxy, aryloxy, cyano, nitro, amino, mono- or di-(C₁-C₆)alkyl amino, oxo (═O), sulfo (═S), imino (═NH), alkylimino (═N(C₁-C₆)alkyl), amidino or oximino (═N—OH) moieties.

Alkyl, alkenyl and alkynyl components of X, Z, R¹, R², R^{a}, R^{b} and R^{c} each may be straight, particularly having 1-6 carbon atoms or branched or cyclic; particularly having 3-6 carbon atoms.

W represents a linking group. W is suitably selected from —(C═O)—, —C(═O)NR^{a}—, —NR^{a}C(═O)—, —C(═O)(CH₂)ₙNR^{a}C(═O)—, C(═S)NR^{a}—, —C(═O)CH₂O—, —SO₂NR^{a}—, —NR^{a}SO₂—, —CH₂NR^{a}—, —C(═O)CH₂—, —CH₂C(═O)— or 5-membered heterocycles;

When W is a five-membered heterocycle, it may be for example, pyrrole, thiophene, furan, imidazole, thiazole, oxazole, pyrazole, isothiazole, isoxazole, 1,2,3-triazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole or 1,3,4-oxadiazole.

Particularly, W is selected from —C(═O)NR^{a}—, —C(═O)(CH₂)ₙNR^{a}C(═O)—, and —C(═O)CH₂—. Particularly R^{a} is —H. n is an integer selected from 0, 1, 2, 3 and 4.

Y represents a second linking group. Y is suitably selected from —H₂—, —O—, —S—, —S(═O)—, —C(═O)—, —SO₂—, —N(R)—, —N(R^{b})SO₂—, —SO₂NR^{b}—, or a single bond;

Particular compounds of the present invention include, but are not limited to, the following compositions:
1-[5-Methoxy-8-(4-methyl-piperazin-1-yl)-3,4-dihydro-1H-isoquinolin-2-yl]-2-(4-piperidin-1-ylmethyl-phenyl)-ethanone;
2-(4-Isopropyl-phenyl)-1-[8-(4-methyl-piperazin-1-yl)-3,4-dihydro-1H-isoquinolin-2-yl]-ethanone;
8-(4-Methyl-piperazin-1-yl)-3,4-dihydro-1H-isoquinoline-2-carboxylic acid (4-morpholin-4-yl-phenyl)-amide;
5-Methoxy-8-phenyl-3,4-dihydro-1H-isoquinoline-2-carboxylic acid (4-morpholin-4-yl-phenyl)-amide;
1-[5-Benzyloxy-8-(4-methyl-piperazin-1-yl)-3,4-dihydro-1H-isoquinolin-2-yl]-2-(4-isopropyl-phenyl)-ethanone;
1-[5-Hydroxy-8-(4-methyl-piperazin-1-yl)-3,4-dihydro-1H-isoquinolin-2-yl]-2-(4-isopropyl-phenyl)-ethanone;
2-(4-Isopropyl-phenyl)-1-(5-methoxy-8-pyridin-4-yl-3,4-dihydro-1H-isoquinolin-2-yl)-ethanone;
2-(4-Isopropyl-phenyl)-1-[5-methoxy-8-(1-methyl-1,2,3,6-tetrahydro-pyridinyl)-3,4-dihydro-1H-isoquinolin-2-yl]-ethanone;
4-{2-[5-Methoxy-8-(4-methyl-piperazin-1-yl)-6-propyl-3,4-dihydro-1H-isoquinolin-2-yl]-2-oxo-ethyl}-N-propyl-benzenesulfonamide;
4-{2-[5-Methoxy-8-(4-methyl-piperazin-1-yl)-3,4-dihydro-1H-isoquinolin-2-yl]-2-oxo-ethyl}-N-propyl-benzenesulfonamide;
N-Isopropyl-4-{2-[5-methoxy-8-(4-methyl-piperazin-1-yl)-3,4-dihydro-1H-isoquinolin-2-yl]-2-oxo-ethyl}-benzenesulfonamide;
N-tert-Butyl-4-{2-[5-methoxy-8-(4-methyl-piperazin-1-yl)-3,4-dihydro-1H-isoquinolin-2-yl]-2-oxo-ethyl}-benzenesulfonamide;
N-Benzyl-4-{2-[5-methoxy-8-(4-methyl-piperazin-1-yl)-3,4-dihydro-1H-isoquinolin-2-yl]-2-oxo-ethyl}-benzenesulfonamide;
N-(2-Methoxy-benzyl)-4-{2-[5-methoxy-8-(4-methyl-piperazin-1-yl)-3,4-dihydro-1H-isoquinolin-2-yl]-2-oxo-ethyl}-benzenesulfonamide;
N-(3-Methoxy-benzyl)₄-{2-[5-methoxy-8-(4-methyl-piperazin-1-yl)-3,4-dihydro-1H-isoquinolin-2-yl]-2-oxo-ethyl}-benzenesulfonamide;
N-(4-Methoxy-benzyl)₄-{2-[5-methoxy-8-(4-methyl-piperazin-1-yl)-3,4-dihydro-1H-isoquinolin-2-yl]-2-oxo-ethyl}-benzenesulfonamide;
4-{2-[5-Methoxy-8-(4-methyl-piperazin-1-yl)-3,4-dihydro-1H-isoquinolin-2-yl]-2-oxo-ethyl}-H-(2-methoxy-phenyl)-benzenesulfonamide;
4-{2-[5-Methoxy-8-(4-methyl-piperazin-1-yl)-3,4-dihydro-1H-isoquinolin-2-yl]-2-oxo-ethyl}-H-(3-methoxy-phenyl)-benzenesulfonamide;
4-{2-[5-Methoxy-8-(4-methyl-piperazin-1-yl)-3,4-dihydro-1H-isoquinolin-2-yl]-2-oxo-ethyl}-H-(4-methoxy-phenyl)-benzenesulfonamide;
N-Cyclopropyl-4-{2-[5-methoxy-8-(4-methyl-piperazin-1-yl)-3,4-dihydro-1H-isoquinolin-2-yl]-2-oxo-ethyl}-benzenesulfonamide;
N-Cyclobutyl-4-{2-[5-methoxy-8-(4-methyl-piperazin-1-yl)-3,4-dihydro-1H-isoquinolin-2-yl]-2-oxo-ethyl}-benzenesulfonamide;
4-{2-[5-Methoxy-8-(4-methyl-piperazin-1-yl)-3,4-dihydro-1H-isoquinolin-2-yl]-2-oxo-ethyl}-benzenesulfonamide;
4-{2-[5-Methoxy-8-(4-methyl-piperazin-1-yl)-3,4-dihydro-1H-isoquinolin-2-yl]-2-oxo-ethyl}-N-methyl-benzenesulfonamide;
4-{2-[5-Methoxy-8-(4-methyl-piperazin-1-yl)-3,4-dihydro-1H-isoquinolin-2-yl]-2-oxo-ethyl}-N-ethyl-benzenesulfonamide;
4-{2-[S-Methoxy-8-(4-methyl-piperazin-1-yl)-3,4-dihydro-1H-isoquinolin-2-yl]-2-oxo-ethyl}-N,N-dimethyl-benzenesulfonamide;
N-Ethyl-4-{2-[5-methoxy-8-(4-methyl-piperazin-1-yl)-3,4-dihydro-1H-isoquinolin-2-yl]-2-oxo-ethyl}-N-methyl-benzenesulfonamide;
N,N-Diethyl-4-{2-[S-methoxy-8-(4-methyl-piperazin-1-yl)-3,4-dihydro-1H-isoquinolin-2-yl]-2-oxo-ethyl}-benzenesulfonamide;
N,N-Dipropyl-4-{2-[5-methoxy-8-(4-methyl-piperazin-1-yl)-3,4-dihydro-1H-isoquinolin-2-yl]-2-oxo-ethyl}-benzenesulfonamide;
N-Benzyl-4-{2-[S-methoxy-8-(4-methyl-piperazin-1-yl)-3,4-dihydro-1H-isoquinolin-2-yl]-2-oxo-ethyl}-N-methyl-benzenesulfonamide;
N-Benzyl-4-{2-[5-methoxy-8-(4-methyl-piperazin-1-yl)-3,4-dihydro-1H-isoquinolin-2-yl]-2—oxo-ethyl}-N-ethyl-benzenesulfonamide;
N-Benzyl-4-{2-[5-methoxy-8-(4-methyl-piperazin-1-yl)-3,4-dihydro-1H-isoquinolin-2-yl]-2-oxo-ethyl}-N-isopropyl-benzenesulfonamide;
2-[4-(Azetidine-1-sulfonyl)-phenyl]-1-[5-methoxy-8-(4-methyl-piperazin-1-yl)-3,4-dihydro-1H-isoquinolin-2-yl]-ethanone;
1-[5-Methoxy-8-(4-methyl-piperazin-1-yl)-3,4-dihydro-1H-isoquinolin-2-yl]-2-[4-(pyrrolidine-1-sulfonyl)-phenyl]-ethanone;
N-{2-[S-Methoxy-8-(4-methyl-piperazin-1-yl)-3,4-dihydro-1H-isoquinolin-2-yl]-2-oxo-ethyl}-isonicotinamide;
N-{4-[5-Methoxy-8-(4-methyl-piperazin-1-yl)-3,4-dihydro-1H-isoquinolin-2-yl]-4-oxo-butyl}-isonicotinamide;
N-{5-[5-Methoxy-8-(4-methyl-piperazin-1-yl)-3,4-dihydro-1H-isoquinolin-2-yl]-5-oxo-pentyl}-isonicotinamide;
Quinoline-5-carboxylic acid {2-[5-methoxy-8-(4-methyl-piperazin-1-yl)-3,4-dihydro-1H-isoquinolin-2-yl]-2-oxo-ethyl}-amide;
Quinoline-5-carboxylic acid {4-[5-methoxy-8-(4-methyl-piperazin-1-yl)-3,4-dihydro-1H-isoquinolin-2-yl]-4-oxo-butyl}-amide;
Quinoline-5-carboxylic acid {5-[5-methoxy-8-(4-methyl-piperazin-1-yl)-3,4-dihydro-1H-isoquinolin-2-yl]-5-oxo-pentyl}-amide;
N-{2-[5-Methoxy-8-(4-methyl-piperazin-1-yl)-3,4-dihydro-1H-isoquinolin-2-yl]-2-oxo-ethyl}-benzamide;
N-{3-[5-Methoxy-8-(4-methyl-piperazin-1-yl)-3,4-dihydro-1H-isoquinolin-2-yl]-3-oxo-propyl}-benzamide;
N-{4-[5-Methoxy-8-(4-methyl-piperazin-1-yl)-3,4-dihydro-1H-isoquinolin-2-yl]-4-oxo-butyl}-benzamide;
N-{5-[5-Methoxy-8-(4-methyl-piperazin-1-yl)-3,4-dihydro-1H-isoquinolin-2-yl]-5-oxo-pentyl}-benzamide;
4-Methoxy-N-{2-[5-methoxy-8-(4-methyl-piperazin-1-yl)-3,4-dihydro-1H-isoquinolin-2-yl]-2-oxo-ethyl}-benzamide;
4-Methoxy-N-{4-[5-methoxy-8-(4-methyl-piperazin-1-yl)-3,4-dihydro-1H-isoquinolin-2-yl]-4-oxo-butyl}-benzamide;
4-Methoxy-N-{5-[5-methoxy-8-(4-methyl-piperazin-1-yl)-3,4-dihydro-1H-isoquinolin-2-yl]-5-oxo-pentyl}-benzamide;
(4-Butylamino-phenyl)-[5-methoxy-8-(4-methyl-piperazin-1-yl)-3,4-dihydro-1H-isoquinolin-2-yl]-methanone;
(4-Cyclohexyl-phenyl)-[5-methoxy-8-(4-methyl-piperazin-1-yl)-3,4-dihydro-1H-isoquinolin-2-yl]-methanone;
(4-Benzyl-phenyl)-[5-methoxy-8-(4-methyl-piperazin-1-yl)-3,4-dihydro-1H-isoquinolin-2-yl]-methanone;
(4′-Ethyl-biphenyl-4-yl)-[5-methoxy-8-(4-methyl-piperazin-1-yl)-3,4-dihydro-1H-isoquinolin-2-yl]-methanone;
(4′-Hydroxy-biphenyl-4-yl)-[5-methoxy-8-(4-methyl-piperazin-1-yl)-3,4-dihydro-1H-isoquinolin-2-yl]-methanone;
[5-Methoxy-8-(4-methyl-piperazin-1-yl)-3,4-dihydro-1H-isoquinolin-2-yl]-(4-phenoxy-phenyl)-methanone;
(4-Benzoyl-phenyl)-[5-methoxy-8-(4-methyl-piperazin-1-yl)-3,4-dihydro-1H-isoquinolin-2-yl]-methanone;
5-Methoxy-8-(4-methyl-piperazin-1-yl)-3,4-dihydro-1H-isoquinoline-2-carboxylic acid [4-(4-methoxy-phenylsulfamoyl)-phenyl]-amide;
5-Methoxy-8-(4-methyl-piperazin-1-yl)-3,4-dihydro-1H-isoquinoline-2-carboxylic acid (4-phenylsulfamoyl-phenyl)-amide;
5-Methoxy-8-(4-methyl-piperazin-1-yl)-3,4-dihydro-1H-isoquinoline-2-carboxylic acid [4-(2-methoxy-phenylsulfamoyl)-phenyl]-amide;
5-Methoxy-8-(4-methyl-piperazin-1-yl)-3,4-dihydro-1H-isoquinoline-2-carboxylic acid [4-(3-methoxy-phenylsulfamoyl)-phenyl]-amide;
5-Methoxy-8-(4-methyl-piperazin-1-yl)-3,4-dihydro-1H-isoquinoline-2-carboxylic acid (4-benzylsulfamoyl-phenyl)-amide;
5-Methoxy-8-(4-methyl-piperazin-1-yl)-3,4-dihydro-1H-isoquinoline-2-carboxylic acid [4-(2-methoxy-benzylsulfamoyl)-phenyl]-amide;
5-Methoxy-8-(4-methyl-piperazin-1-yl)-3,4-dihydro-1H-isoquinoline-2-carboxylic acid [4-(3-methoxy-benzylsulfamoyl)-phenyl]-amide;
5-Methoxy-8-(4-methyl-piperazin-1-yl)-3,4-dihydro-1H-isoquinoline-2-carboxylic acid [4-(4-methoxy-benzylsulfamoyl)-phenyl]-amide;
5-Methoxy-8-(4-methyl-piperazin-1-yl)-3,4-dihydro-1H-isoquinoline-2-carboxylic acid (4-propylsulfamoyl-phenyl)-amide;
5-Methoxy-8-(4-methyl-piperazin-1-yl)-3,4-dihydro-1H-isoquinoline-2-carboxylic acid (4-isopropylsulfamoyl-phenyl)-amide;
5-Methoxy-8-(4-methyl-piperazin-1-yl)-3,4-dihydro-1H-isoquinoline-2-carboxylic acid (4-cyclopropylsulfamoyl-phenyl)-amide,
5-Methoxy-8-(4-methyl-piperazin-1-yl)-3,4-dihydro-1H-isoquinoline-2-carboxylic acid (4-tert-butylsulfamoyl-phenyl)-amide;
5-Methoxy-8-(4-methyl-piperazin-1-yl)-3,4-dihydro-1H-isoquinoline-2-carboxylic acid (4-methylsulfamoyl-phenyl)-amide;
5-Methoxy-8-(4-methyl-piperazin-1-yl)-3,4-dihydro-1H-isoquinoline-2-carboxylic acid (4-ethylsulfamoyl-phenyl)-amide;
5-Methoxy-8-(4-methyl-piperazin-1-yl)-3,4-dihydro-1H-isoquinoline-2-carboxylic acid (4-cyclobutylsulfamoyl-phenyl)-amide;
5-Methoxy-8-(4-methyl-piperazin-1-yl)-3,4-dihydro-1H-isoquinoline-2-carboxylic acid [4-(thiazol-2-ylsulfamoyl)-phenyl]-amide;
5-Methoxy-8-(4-methyl-piperazin-1-yl)-3,4-dihydro-1H-isoquinoline-2-carboxylic acid (4-acetylsulfamoyl-phenyl)-amide;
5-Methoxy-8-(4-methyl-piperazin-1-yl)-3,4-dihydro-1H-isoquinoline-2-carboxylic acid (4-butyrylsulfamoyl-phenyl)-amide;
5-Methoxy-8-(4-methyl-piperazin-1-yl)-3,4-dihydro-1H-isoquinoline-2-carboxylic acid [4-(methyl-phenyl-sulfamoyl)-phenyl]-amide;
5-Methoxy-8-(4-methyl-piperazin-1-yl)-3,4-dihydro-1H-isoquinoline-2-carboxylic acid [4-(acetyl-methyl-sulfamoyl)-phenyl]-amide;
1-[5-Methoxy-8-(4-methyl-piperazin-1-yl)-3,4-dihydro-1H-isoquinolin-2-yl]-2-(4-morpholin-4-yl-phenyl)-ethanone;
2-(4-Bromo-phenyl)-1-[5-methoxy-8-(4-methyl-piperazin-1-yl)-3,4-dihydro-1H-isoquinolin-2-yl]-ethanone;
2-(4-Dimethylamino-phenyl)-1-[5-methoxy-8-(4-methyl-piperazin-1-yl)-3,4-dihydro-1H-isoquinolin-2-yl]-ethanone;
1-[5-Methoxy-8-(4-methyl-piperazin-1-yl)-3,4-dihydro-1H-isoquinolin-2-yl]-2-(3-morpholin-4-yl-phenyl)-ethanone;
1-[5-Methoxy-8-(4-methyl-piperazin-1-yl)-3,4-dihydro-1H-isoquinolin-2-yl]-2-(4-piperidin-1-yl-phenyl)-ethanone;
1-[5-Methoxy-8-(4-methyl-piperazin-1-yl)-3,4-dihydro-1H-isoquinolin-2-yl]-2-[4-(4-methyl-piperazin-1-yl)-phenyl]-ethanone;
1-[5-Methoxy-8-(4-methyl-piperazin-1-yl)-3,4-dihydro-1H-isoquinolin-2-yl]-2-[4-(4-propyl-piperidin-1-yl)-phenyl]-ethanone;
2-{4-[4-(2-Methoxy-ethyl)-piperidin-1-yl]-phenyl}-1-[5-methoxy-8-(4-methyl-piperazin-1-yl)-3,4-dihydro-1H-isoquinolin-2-yl]-ethanone;
1-[5-Methoxy-8-(4-methyl-piperazin-1-yl)-3,4-dihydro-1H-isoquinolin-2-yl]-2-[4-(4-methyl-piperidin-1-yl)-phenyl]-ethanone;
2-[4-(4-Hydroxy-piperidin-1-yl)-phenyl]-1-[5-methoxy-8-(4-methyl-piperazin-1-yl)-3,4-dihydro-1H-isoquinolin-2-yl]-ethanone;
2-{4-[4-(2-Hydroxy-ethyl)-piperazin-1-yl]-phenyl}-1-[5-methoxy-8-(4-methyl-piperazin-1-yl)-3,4-dihydro-1H-isoquinolin-2-yl]-ethanone;
2-(4-Amino-phenyl)-1-[5-methoxy-8-(4-methyl-piperazin-1-yl)-3,4-dihydro-1H-isoquinolin-2-yl]-ethanone;
2-(4-Isopropyl-phenoxy)-1-[5-methoxy-8-(4-methyl-piperazin-1-yl)-3,4-dihydro-1H-isoquinolin-2-yl]-ethanone;
2-[4-(4-Benzyl-piperazin-1-yl)-phenyl]-1-[5-methoxy-8-(4-methyl-piperazin-1-yl)-3,4-dihydro-1H-isoquinolin-2-yl]-ethanone;
2-(4-Isopropyl-phenyl)-1-[S-methoxy-8-(4-methyl-piperazin-1-yl)-3,4-dihydro-1H-isoquinolin-2-yl]-ethanone;
5-Methoxy-8-(4-methyl-piperazin-1-yl)-3,4-dihydro-1H-isoquinoline-2-carboxylic acid (4-thiomorpholin-4-yl-phenyl)-amide;
4-Amino-N-(4-{2-[5-methoxy-8-(4-methyl-piperazin-1-yl)-3,4-dihydro-1H-isoquinolin-2-yl]-2-oxo-ethyl}-phenyl)-butyramide;
2-(4-Dibutylamino-phenyl)-1-[5-methoxy-8-(4-methyl-piperazin-1-yl)-3,4-dihydro-1H-isoquinolin-2-yl]-ethanone;
2-(4-Butylamino-phenyl)-1-[5-methoxy-8-(4-methyl-piperazin-1-yl)-3,4-dihydro-1H-isoquinolin-2-yl)-ethanone;
2-(4-Diphenethylamino-phenyl)-1-[5-methoxy-8-(4-methyl-piperazin-1-yl)-3,4-dihydro-1H-isoquinolin-2-yl]-ethanone;
1-[5-Methoxy-8-(4-methyl-piperazin-1-yl)-3,4-dihydro-1H-isoquinolin-2-yl]-2-(4-phenethylamino-phenyl)-ethanone;
2-{4-[Bis-(2-benzyloxy-ethyl)-amino]-phenyl}-1-[5-methoxy-8-(4-methyl-piperazin-1-yl)-3,4-dihydro-1H-isoquinolin-2-yl]-ethanone;
2-[4-(2-Benzyloxy-ethylamino)-phenyl]-1-[5-methoxy-8-(4-methyl-piperazin-1-yl)-3,4-dihydro-1H-isoquinolin-2-yl]-ethanone;
Biphenyl-4-yl-[5-methoxy-8-(4-methyl-piperazin-1-yl)-3,4-dihydro-1H-isoquinolin-2-yl]-methanone;
2-Biphenyl-4-yl-1-[5-methoxy-8-(4-methyl-piperazin-1-yl)-3,4-dihydro-1H-isoquinolin-2-yl]-ethanone;
1-(5-Methoxy-8-(4-methyl-piperazin-1-yl)-3,4-dihydro-1H-isoquinolin-2-yl]-2-(4-methoxy-phenyl)-ethanone;
2-Benzo[1,3]dioxol-5-yl-1-[5-methoxy-8-(4-methyl-piperazin-1-yl)-3,4-dihydro-1H-isoquinolin-2-yl]-ethanone;
2-(3,4-Dimethoxy-phenyl)-1-[5-methoxy-8-(4-methyl-piperazin-1-yl)-3,4-dihydro-1H-isoquinolin-2-yl]-ethanone;
2-(4-Fluoro-phenyl)-1-[5-methoxy-8-(4-methyl-piperazin-1-yl)-3,4-dihydro-1H-isoquinolin-2-yl]-ethanone;
2-(4-Chloro-phenyl)-1-[5-methoxy-8-(4-methyl-piperazin-1-yl)-3,4-dihydro-1H-isoquinolin-2-yl]-ethanone;
2-(4-methyl-phenyl)-1-[5-methoxy-8-(4-methyl-piperazin-1-yl)-3,4-dihydro-1H-isoquinolin-2-yl]-ethanone;
2-Phenyl-1-[5-methoxy-8-(4-methyl-piperazin-1-yl)-3,4-dihydro-1H-isoquinolin-2-yl]-ethanone;
1-[5-Methoxy-8-(4-methyl-piperazin-1-yl)-3,4-dihydro-1H-isoquinolin-2-yl]-2-(4-methylsulfanyl-phenyl)-ethanone;
2-(4-Methanesulfinyl-phenyl)-1-[5-methoxy-8-(4-methyl-piperazin-1-yl)-3,4-dihydro-1H-isoquinolin-2-yl]-ethanone;
N-(4-{2-[5-Methoxy-8-(4-methyl-piperazin-1-yl)-3,4-dihydro-1H-isoquinolin-2-yl]-2-oxo-ethyl}-phenyl)-methanesulfonamide;
2-[4-(2-Methoxy-benzylamino)-phenyl]-1-[5-methoxy-8-(4-methyl-piperazin-1-yl)-3,4-dihydro-1H-isoquinolin-2-yl]-ethanone;
2-(4-Benzylamino-phenyl)-1-[5-methoxy-8-(4-methyl-piperazin-1-yl)-3,4-dihydro-1H-isoquinolin-2-yl]-ethanone;
2-[4-(3-Methoxy-benzylamino)-phenyl]-1-[5-methoxy-8-(4-methyl-piperazin-1-yl)-3,4-dihydro-1H-isoquinolin-2-yl]-ethanone;
2-[4-(4-Methoxy-benzylamino)-phenyl]-1-[5-methoxy-8-(4-methyl-piperazin-1-yl)-3,4-dihydro-1H-isoquinolin-2-yl]-ethanone;
2-(4-Isopropyl-phenyl)-1-[5-methoxy-8-(4-methyl-piperazine-1-carbonyl)-3,4-dihydro-1H-isoquinolin-2-yl]-ethanone;
5-Methoxy-8-(4-methyl-piperazin-1-yl)-3′,4-dihydro-1H-isoquinoline-2-carboxylic acid (4-isopropyl-phenyl)-amide;
5-Methoxy-8-(4-methyl-piperazin-1-yl)-3,4-dihydro-1H-isoquinoline-2-carboxylic acid (4-cyclohexyl-phenyl)-amide;
5-Methoxy-8-(4-methyl-piperazin-1-yl)-3,4-dihydro-1H-isoquinoline-2-carboxylic acid [4-(5-methoxy-pyrimidin-2-ylsulfamoyl)-phenyl]-amide;
(4-{[5-Methoxy-8-(4-methyl-piperazin-1-yl)-3,4-dihydro-1H-isoquinoline-2-carbonyl]-amino)-benzyl)-phosphonic acid diethyl ester;
5-Methoxy-8-(4-methyl-piperazin-1-yl)-3,4-dihydro-1H-isoquinoline-2-carboxylic acid [4-(3,4-dimethyl-isoxazol-5-ylsulfamoyl)-phenyl]-amide;
5-Methoxy-8-(4-methyl-piperazin-1-yl)-3,4-dihydro-1H-isoquinoline-2-carboxylic acid [4-(6-methyl-benzothiazol-2-yl)-phenyl]-amide;
5-Methoxy-8-(4-methyl-piperazin-1-yl)-3,4-dihydro-1H-isoquinoline-2-carboxylic acid (4-#tert!-butyl-phenyl)-amide;
5-Methoxy-8-(4-methyl-piperazin-1-yl)-3,4-dihydro-1H-isoquinoline-2-carboxylic acid (4-sulfamoyl-phenyl)-amide;
5-Methoxy-8-(4-methyl-piperazin-1-yl)-3,4-dihydro-1H-isoquinoline-2-carboxylic acid [4-(2-phenyl-2H-pyrazol-3-ylsulfamoyl)-phenyl]-amide;
5-Methoxy-8-(4-methyl-piperazin-1-yl)-3,4-dihydro-1H-isoquinoline-2-carboxylic acid [4-(pyrrolidine-1-sulfonyl)-phenyl]-amide;
5-Methoxy-8-(4-methyl-piperazin-1-yl)-3,4-dihydro-1H-isoquinoline-2-carboxylic acid [4-(5-methyl-[1,3,4]thiadiazol-2-ylsulfamoyl)-phenyl]-amide;
5-Methoxy-8-(4-methyl-piperazin-1-yl)-3,4-dihydro-1H-isoquinoline-2-carboxylic acid [4-(4,5-dimethyl-oxazol-2-ylsulfamoyl)-phenyl]-amide;
5-Methoxy-8-(4-methyl-piperazin-1-yl)-3,4-dihydro-1H-isoquinoline-2-carboxylic acid [4-(2-phenyl-2H-pyrazol-3-ylsulfamoyl)-phenyl]-amide;
5-Methoxy-8-(4-methyl-piperazin-1-yl)-3,4-dihydro-1H-isoquinoline-2-carboxylic acid [4-(4-methyl-pyrimidin-2-ylsulfamoyl)-phenyl]-amide;
5-Methoxy-8-(4-methyl-piperazin-1-yl)-3,4-dihydro-1H-isoquinoline-2-carboxylic acid [4-(2,6-dimethyl-pyrimidin-4-ylsulfamoyl)-phenyl]-amide;
5-Methoxy-8-(4-methyl-piperazin-1-yl)-3,4-dihydro-1H-isoquinoline-2-carboxylic acid [4-(pyrimidin-2-ylsulfamoyl)-phenyl]-amide;
5-Methoxy-8-(4-methyl-piperazin-1-yl)-3,4-dihydro-1H-isoquinoline-2-carboxylic acid [4-(2,6-dimethoxy-pyrimidin-4-ylsulfamoyl)-phenyl]-amide;
5-Methoxy-8-(4-methyl-piperazin-1-yl)-3,4-dihydro-1H-isoquinoline-2-carboxylic acid [4-(6-methoxy-pyridazin-3-ylsulfamoyl)-phenyl)-amide;
5-Methoxy-8-(4-methyl-piperazin-1-yl)-3,4-dihydro-1H-isoquinoline-2-carboxylic acid [4-(4,6-dimethyl-pyrimidin-2-ylsulfamoyl)-phenyl]-amide;
5-Methoxy-8-(4-methyl-piperazin-1-yl)-3,4-dihydro-1H-isoquinoline-2-carboxylic acid [4-(6-methoxy-pyrimidin-4-ylsulfamoyl)-phenyl]-amide;
5-Methoxy-8-(4-methyl-piperazin-1-yl)-3,4-dihydro-1H-isoquinoline-2-carboxylic acid [4-(pyridin-2-ylsulfamoyl)-phenyl]-amide;
4-{2-[5-Methoxy-8-(4-methyl-piperazin-1-yl)-3,4-dihydro-1H-isoquinolin-2-yl]-2-oxo-ethyl}-benzoic acid methyl ester;
4-{2-[5-Methoxy-8-(4-methyl-piperazin-1-yl)-3,4-dihydro-1H-isoquinolin-2-yl]-2-oxo-ethyl}-N-methyl-benzamide;
8-(4-Ethyl-piperazin-1-yl)-5-methoxy-3,4-dihydro-1H-isoquinoline-2-carboxylic acid (4-propylsulfamoyl-phenyl)-amide;
8-(4-Cyclohexyl-piperazin-1-yl)-5-methoxy-3,4-dihydro-1H-isoquinoline-2-carboxylic acid (4-propylsulfamoyl-phenyl)-amide;
2-(4-Isopropyl-phenyl)-1-(5-methoxy-8-piperazin-1-yl-3,4-dihydro-1H-isoquinolin-2-yl)-ethanone;
N-(4-{2-[5-Methoxy-8-(4-methyl-piperazin-1-yl)-3,4-dihydro-1H-isoquinolin-2-yl]-2-oxo-ethyl}-phenyl)-2-phenyl-acetamide;
N-(4-{2-[5-Methoxy-8-(4-methyl-piperazin-1-yl)-3,4-dihydro-1H-isoquinolin-2-yl]-2-oxo-ethyl}-phenyl)-3-phenyl-propionamide;
N-(4-{2-[5-Methoxy-8-(4-methyl-piperazin-1-yl)-3,4-dihydro-1H-isoquinolin-2-yl]-2-oxo-ethyl}-phenyl)-benzamide;
N-(4-{2-[5-Methoxy-8-(4-methyl-piperazin-1-yl)-3,4-dihydro-1H-isoquinolin-2-yl]-2-oxo-ethyl}-phenyl)-benzenesulfonamide;
1-[5-Methoxy-8-(4-methyl-piperazin-1-yl)-3,4-dihydro-1H-isoquinolin-2-yl]-2-(4-phenylmethanesulfonyl-methyl-phenyl)-ethanone;
4-Chloro-N-(4-{2-[5-methoxy-8-(4-methyl-piperazin-1-yl)-3,4-dihydro-1H-isoquinolin-2-yl]-2-oxo-ethyl}-phenyl)-benzenesulfonamide;
4-tert-Butyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide;
N-Benzyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide;
1-(4-{2-[5-Methoxy-8-(4-methyl-piperazin-1-yl)-3,4-dihydro-1H-isoquinolin-2-yl]-2-oxo-ethyl}-phenyl)-3-(4-methoxy-phenyl)-urea;
1-(4-{2-[5-Methoxy-8-(4-methyl-piperazin-1-yl)-3,4-dihydro-1H-isoquinolin-2-yl]-2-oxo-ethyl}-phenyl)-3-(3-methoxy-phenyl)-urea;
[5-Methoxy-8-(4-methyl-piperazin-1-yl)-3,4-dihydro-1H-isoquinolin-2-yl]-[2′-methyl-4′-(5-methyl-[1,2,4]oxadiazol-3-yl)-biphenyl-4-yl]-methanone;
5-Methoxy-8-(4-methyl-piperazin-1-yl)-3,4-dihydro-1H-isoquinoline-2-carboxylic acid (4-piperidin-1-yl-phenyl)-amide; and
5-Methoxy-8-(4-methyl-piperazin-1-yl)-3,4-dihydro-1H-isoquinoline-2-carboxylic acid [4-(4-methyl-piperazin-1-yl)-phenyl]-amide.

The compounds provided herein are useful in the form as a free base, but may also be provided in the form of a pharmaceutically acceptable salt, and/or in the form of a pharmaceutically acceptable hydrate. For example, pharmaceutically acceptable salts of compounds of Formula I include those derived from mineral acids such as for example: hydrochloric acid, nitric acid, phosphoric acid, sulfuric acid, hydrobromic acid, hydroiodic acid, nitrous acid, and phosphorous acid. Pharmaceutically acceptable salts may also be developed with organic acids including aliphatic mono and dicarboxylates and aromatic acids. Other pharmaceutically-acceptable salts of compounds of the present invention include for example hydrochloride, sulfate, pyrosulfate, bisulfate, bisulfite, nitrate, and phosphate.

Processes for the manufacture of the compounds of Formula I are provided as further features of the invention. Many of the Compounds described herein can be made by processes known in the chemical arts for the production of structurally analogous compounds. Accordingly, the compounds of this invention may be prepared by employing procedures known in the literature starting from known compounds or readily prepared intermediates.

For compounds of the present invention that have W as an alkanoyl or aroyl moiety forming an amide bond with the isoquinoline nitrogen, the compounds are particularly made by the general procedure for amide coupling, that is by coupling an anime with an activated carboxylic acid such as an acid halide; for example an acid chloride.

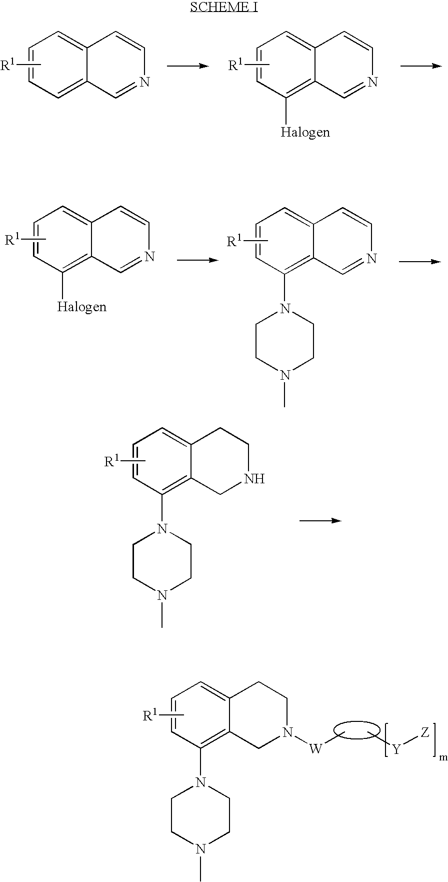

It will be appreciated by those skilled in the art that certain compounds of the present invention contain for example asymmetrically substituted carbon and/or sulfur atoms, and accordingly may exist in and be isolated in, optically-active and racemic forms. Some compounds may exhibit polymorphism, thus it is to be understood that the present invention encompasses racemic, optically-active, polymorphic or stereoisomeric forms, or mixtures thereof, which form, possess properties useful in the treatment of the disorders set forth below, it being well known in the art how to prepare optically-active forms (for example by resolution of the racemic forms by recrystallization techniques, by synthesis from optically-active starting materials, by chiral synthesis, or by chromatographic separation using a chiral stationary phase) and how to determine efficacy for the treatment of the disorder described above.

Compounds of Formula I have been found by the inventors to be useful as 5-HT_{1B} and 5HT_{1D} antagonists. The compounds of Formula I, and their pharmaceutically acceptable salts, may also be used in a method for the treatment of depression, generalized anxiety, eating disorders, dementia, panic disorder, sleep disorders, gastrointestinal disorders, motor disorders, endocrine disorders, vasospasm and sexual dysfunction. The treatment of these disorders comprises administering to a warm-blooded animal, particularly a mammal, more particularly a human, in need of such treatment, an effective amount of a compound of Formula I, or a pharmaceutically acceptable salt of said compound.

Compounds of formula I have also been found to be 5-HT_{1B} and 5HT_{1D} agonists. The compounds of Formula I, and their pharmaceutically acceptable salts, may also be used in a method for the treatment of migraine. The treatment of this disorder comprises administering to a warm-blooded animal, particularly a mammal, more particularly a human, in need of such treatment, an effective amount of a compound of Formula I or a pharmaceutically acceptable salt of said compound.

Further provided herein are compounds of Formula I, and their pharmaceutically acceptable salts, for use in the treatment of depression, generalized anxiety, eating disorders, dementia, panic disorder, sleep disorders, gastrointestinal disorders, motor disorders, endocrine disorders, vasospasm and sexual dysfunction of an animal, particularly a mammal, most particularly a human, in need of such therapy.

Further provided herein is a method of treatment of a warm-blooded animal, particularly a mammal, most particularly a human, suffering from depression, generalized anxiety, eating disorders, dementia, panic disorder, sleep disorders, gastrointestinal disorders, motor disorders, endocrine disorders, vasospasm or sexual dysfunction, comprising administering to such animal an effective amount of a compound of Formula I, or a pharmaceutically acceptable salt of the compound.

Further provided is the use of a compound of Formula I in the preparation of a medicant for the treatment of a disorder such as depression, generalized anxiety, eating disorders, dementia, panic disorder, sleep disorders, gastrointestinal disorders, motor disorders, endocrine disorders, vasospasm and sexual dysfunction in warm-blooded animal, particularly a mammal, most particularly a human, suffering from such disorder.

Further provided is the use of a compound of Formula I in the preparation of a medicament for the treatment of a disorder such as migraine in a warm-blooded animal, particularly a mammal, more particularly a human, suffering from such disorder.

The invention further provides a pharmaceutical composition suitable for the treatment of the above describe disorders comprising administering to a warm-blooded animal having such disorder an effective amount of a pharmaceutical composition of a compound of Formula I, a pharmaceutically acceptable salt.

The invention also provides a pharmaceutical composition comprising a compound of Formula I, as defined herein, or a pharmaceutically acceptable salt, in combination with a pharmaceutically acceptable carrier. Particular compounds of Formula I for use in the compositions of the invention are as described above.

The compounds described herein may be provided or delivered in a form suitable for oral use, for example in a tablet, lozenges, hard and soft capsule, aqueous solutions, oily solutions, emulsions, and suspensions. The compounds may be also be provided for topical administration, for example, as a cream, ointment, gel, spray, or aqueous solutions, oily solutions, emulsions or suspensions. The compounds described herein may also be provided in a form suitable for nasal administration for example, as a nasal spray, nasal drops, or dry powder. The compositions may also be administered to the vagina or rectum in the form of a suppository. The compounds described herein may also be administered parentally, for example by intravenous, intravesicular, subcutaneous, or intramuscular injection or infusion. The compounds may be administered by insufflation (for example as a finely divided powder). The compounds may also be administered transdermally or sublingually.

The compositions of the invention may accordingly be obtained by conventional procedures using conventional pharmaceutical excipients, well known in the art. Thus, compositions intended for oral use may contain, for example, one or more coloring, sweetening, flavoring and/or preservative agents.

The amount of active ingredient that is combined with one or more excipients to produce a single dosage form will necessarily vary depending upon the host treated and the particular route of administration. The size of the dose for therapeutic or prophylactic purposes of a compound of the Formula I, will naturally vary according to the nature and severity of the conditions, the age and sex of the animal or patient and the route of administration, according to well known principles of medicine. Various assays and in vivo tests are known for determining the utility of the compounds in the disorders noted above and specifically as agonists and antagonists of 5HT_{1B} and 5HT_{1D}

The utility of the compounds for example to treat depression may be shown via a learned helplessness test in guinea pigs. The learned helplessness test may be carried out as follows: Seventy male Hartley guinea pigs, each weighing about 350-425 gm are fed ad lib, and are housed under a 12-hour light/dark cycle. The procedure comprises two phases: The induction phase and the avoidance training phase. In the induction phase, subjects are placed into standard shuttle cages (20 L×16 W×21 centimeters H) which are fitted with a grid floor. Electrical stimulation (1.25 mA, 10 sec duration) is delivered to the floor of the cage every 90-sec during 1 hour daily sessions. Subjects have no opportunity to escape or to avoid shocks. Induction is conducted for two consecutive days.

In avoidance training, testing is also conducted in the shuttle cages, except that the subjects are not returned to the same chamber in which induction had occurred. Additionally, all cages are fitted with a partition with and arch in the center of the cage, through which animals can pass between the left and right halves of the cage. The procedure employed is a standard shuttle avoidance procedure in which a compound, conditioned stimulus (a 10-sec presentation of a tone and turning on of a lamp on the side of the cage that the guinea pig was occupying) serves to indicate presentation of electrical current to the floor of the cage. Shock is presented for a 5 sec period, 5 sec after initiation of the conditioned stimulus. Entry into the opposite side of the shuttle cage via the arched partition prior to shock onset results in the end of the trial (avoidance response). If shock is delivered, entry into the opposite side of the cage results in termination of the shock and CS (escape).

Avoidance training, 45-min in duration, is conducted on 2 consecutive days, beginning 48 hr after the final induction session. Seventy subjects are assigned to 1 of 6 groups of 11-12 animals. The groups are as follows:

1) No induction group. The subjects are placed into the shuttle cages but are not given inescapable shock, the animals are subsequently trained in the avoidance procedure and the vehicle is administered;

2) Induction vehicle control group;

3) imipramine 17.8 mg/kg;

4) 0.3 mg/kg compounds;

5) 1 mg/kg compounds; and

6) 5 mg/kg compounds.

Groups 2-6 are given induction and avoidance training sessions. Injections are administered immediately following induction sessions and 1 hour prior to avoidance training sessions. A second injection is administered 7-8 hours following the first injection, for a total of 9 injections administered over 5 days. No injections are administered following the final avoidance training session.

Compounds of the present invention may be administered in a volume of 1 mL/kg bwt. Imipramine is dissolved in DI water. The compounds are dissolved in DI water, to which was added a few drops of lactic acid (pH 5.5). The vehicle control is DI water prepared with lactic acid to the same pH as the-treated groups.

The primary dependent variable is escape failure during avoidance training. 2 way analysis of variance (ANOVA) is used to assess overall treatment effect, with Dunn's post hoc analysis used to compare the vehicle-treated group with the drug-treated groups. The no-induction group is used to gauge whether learned helplessness is established, by comparison to the vehicle treated group.

An alternative method for determining the utility of the compounds of the present invention is to investigate the in vivo activity of the compounds using a guinea pig hypothermia test. Compounds that bind to 5-HT_{1B} receptors are known to be useful in treating disorders described above (e.g., depression, generalized anxiety, eating disorders, dementia, panic disorder, sleep disorders, gastrointestinal disorders, motor disorders, endocrine disorders, vasospasm and sexual dysfunction. While not wishing to be bound to any theory, it is believed that 5-HT_{1B} receptors on nerve terminals control the amount of release of s5-ht into the synapse. Thus, it can be shown that compounds of Formula I, their pharmaceutically acceptable salts, are able to act as 5-HT_{1B} antagonists and block the agonist-induced effect as a method for assessing whether the novel compounds are effective in the treatment of said disorder.

The hypothermia test is conducted as follows: A tele-thermometer fitted with a flexible probe will be used. The tip of the probe is immersed in a test tube containing a lubrication agent between usage. Core temperature is measured by inserting the probe into the rectum and by waiting for the temperature to stabilize, which may occurs within 20-60 seconds. Core temperature is measured once (pretest) prior to administration of the test substance in order to establish a baseline temperature for all animals. Guinea pigs are then dosed with the test substance (candidate 5-ht_{1b} antagonist) either subcutaneously or intraperitoneally. In general, 30 min following dosing with antagonist, agonist is administered subcutaneously. The temperature is then recorded 30-, 60, 90-min following agonist. In some studies, in order to record time course of antagonist activity, up to 12 hours may be allowed to elapse between administration of antagonist and agonist. The drugs may either be injected subcutaneously, intraperitoneally or orally (using a flexible plastic gavage tube, or a stainless steel gavage tube). Animals may be observed on the days following drug administration in order to monitor for unexpected toxicity. The body temperature of the guinea pigs is recorded separately for each guinea pig at each test time point, and submitted to a ANOVA with one between subjects factor: dose, and one within subject factor: time. Following a significant two-way interaction (p<0.05), Dunnett's t-test is performed to compare the drug treatment with either the saline or the effects of treatment with the hypothermic agent.

Male Guinea Pig (Dunkin-Hartley), maximum 3 animals per cage, are used. The animals may be grouped in sets of 5 during testing. The animals will not be deprived of food or water during their time in the laboratory. The routes of administration are: S.C., I.P., P.O. The maximum dose (volume) is 2 mL/kg s.c. or i.p., 5 mL/kg P.O. three times daily.

This method may function as a primary in vivo screen for compounds having an affinity for 5-ht_{1b} receptors. Each experiment may comprise separate groups of 5 subjects per treatment level. One group is given vehicle prior to agonist administration and may serve as the control group. The other groups are administered different doses of antagonist prior to agonist administration, but no more than 5 groups are tested at a time. In order to determine full dose effect functions for compounds (to determine drug potency) 4-6 doses of each compound are evaluated. That results in about 25-35 animals per drug to be evaluated.

Other assays that may be used to measure for example affinity of compounds of the present invention for 5HT_{1B} and 5HT_{1D} receptors are described in J. Med. Chem 41:1218-1235, 1228 (1998) and J. Med. Chem 42:4981-5001, (1999) and incorporated by reference herein. These assays may be used with some modifications: Frozen membrane preparations of a stably transfected CHO cell line expressing 5-HT_{1B} receptors and 5-HT_{1D} receptors are thawed rapidly, briefly vortexed, and diluted in assay buffer (AB) containing 50 mM Tris-HCl, 4 mM MgCl₂, 4 mM CaCl₂, 1 mM EDTA, and adjusted to pH 7.4 with NaOH. Final protein concentrations are—0.185 mg/mL for 5-HT_{1B}, and 0.4 mg/mL for 5-HT_{1D} membranes. Test compounds are evaluated in competition assays using [³H]-GR125743 (Amersham). The ligand concentration in both assays was 0.27 nM. Kd for [³H]-GR125743 may vary from 0.15 nM to 0.25 nM. The 5-HT_{1B} and 5-HT_{1D} assays are performed simultaneously on one 96-well assay plate, one drug/compound per plate. Ten serial dilutions (1 uM to 4 pM, final concentration) of compound are prepared in DMSO from 10 mM stock solutions. Incubation mixtures are prepared in quadruplicate in 96-deep well assay plates (Matrix 1 mL). Final assay volumes per well are 10 μl compound/nonspecific; 100 μl membranes; 100 μl [3H]-GR125743; and 790 μl AB. Specific binding is defined by using 10 uM Methiothepine. The assay plates are shaken for 5 min., and then incubated for an additional 55 min. Then the assay plates are filtered through-Beckman GF/B filters (soaked>2 hrs. in PEI) using a Packard Filtermate 196. Filters are washed 2× with 1 mL ice-cold wash buffer (5 mM Tris-HCl-pH7.4 with NaOH). After the filters are dried, 35 μl of Microscint20 is added to each well. The plates are then counted on a Packard TopCount to determine CPM's per well. Ki values are determined for each test compound utilizing the graphic and analytical software package, GraphPad Prism. Compounds are then ranked in order of potency, and selectivity for 5-HT_{1B} over 5-HT_{1D} receptors.

A method that may be used to determine a compound's affinity for 5-HT_{1B} and 5HT_{1D} receptors is a guinea pig cortical test. The test is carried out as follows:

Guinea pigs are decapitated and the cortici is dissected out, weighed and homogenized in 50 mM Tris-HCl, pH 7.7 with an Ultra-Turrax followed by centrifugation for 10 min at 48000×g and 5° C. The pellet is resuspended and recentrifuged. The final pellet is suspended in 0.32 M sucrose buffer to a concentration of 0.5 g original wet weight per mL and stored frozen at −70° C. The radioligand binding assay is carried out as follows: [³H]GR125743 saturation studies are tested in duplicate with 34 mg w.w. per tube in 5 mL buffer (50 mM Tris, 4 mM CaCl2, 4 mM MgCl2 and 1 mM EDTA at pH 7.7), and a concentration range of 0.012-2 nM (10-12 concentrations) for the radioligand. Non-specific binding is determined in the presence of 10 mM methiothepin. In competition experiments 4-8 mg w.w. per tube and a radioligand concentration of 0.2 nM are used with 10-12 concentrations of the competing drug. The assays are run for ₂₋₄ hours at 30° C. and terminated by rapid filtration through Whatman GF/B filters (pretreated with 0.1% polyethyleneimine) using a Brandel cell harvester. Bovine serum albumin (0.1%) is added to the washing buffer to reduce non-specific binding. Data from the experiments may be analyzed using the iterative non-linear curve-fitting program LIGAND. The K_{d} values obtained from the saturation studies are used in the calculation of the Ki values by the LIGAND program. The K_{d} value of [³H]GR125743 may result in a measurement of 46±4 pM and the Bₘₐₓ in a measurement of 4.9±0.2 pmol/g w.w.

A GTPγS binding assay may used to determine whether a compound is a 5HT_{1B} or 5HT_{1D} agonist. One assay available measures agonist stimulated GTP binding for example as set forth by Lazareno, S. (1999) Methods in Molecular Biology 106: 231-245. Membrane preparations of a stably transfected CHO cell line expressing human 5-HT_{1B} receptors are purchased for example from Unisyn, Hopkinton, Mass. Frozen membranes are thawed, briefly sonicated, and diluted to 167 μg/mL protein in assay buffer containing 20 mM HEPES, 100 mM NaCl, 1 mM MgCL₂ and 1 μM GDP, pH adjusted to 7.4 with NaOH. Diluted membranes are briefly homogenized with a Polytron and allowed to equilibrate at room temperature for at least 15 minutes before use. Serial dilutions (10 μM to 1 pM, final concentration) of test compounds are prepared in buffer with and without 100 nM 5-HT (final concentration) from 10 mM DMSO stock solutions. Incubation mixtures are prepared in quadruplicate in 96-well, deep-well plates and consisted of 180 μL of membranes (30 μg protein) and 40 μL of compound with or without 5-HT. After an incubation period of 15 minutes at room temperature, 20 μL of [³⁵S]GTPγS (NEN; 100 pM final concentration) is added to begin the assay. Mixtures are shaken for 2 minutes and incubated at room temperature for an additional 28 minutes. The reaction is stopped by rapid filtration through Beckman GF/B glass fiber filters using a 96-well Packard cell harvester. Filters are washed four times with 1 mL ice-cold water. The filter plates are nominally dried and 30 μL of scintillation cocktail (MicroScint 40, Packard) is added to each well. CPMs for each well is determined using a TopCount Scintillation Counter (Packard). Maximum stimulation of [³⁵S]GTPγS binding is defined in the presence of 100 nM 5-HT. Basal [³⁵S]GTPγS binding is defined in buffer alone. IC50 values are defined as the concentration of compound at which 50% of the 100 nM 5-HT response [was] obtained. Maximal intrinsic activity (IA) of a compound is defined as the percent maximal 5-HT-induced stimulation by 10 μM compound in the absence of 5-HT. As an inter-assay standard, a concentration response curve of 5-HT (1 μM to 1 pM final) in the absence of compounds was included in each assay and an EC₅₀ was determined.

### EXAMPLES

The following examples illustrate the synthesis of compounds of the present invention, and are not intend to limit the invention in any manner.

The following solvent and reagent abbreviations are employed.

DCM Dichloromethane

HATU O-(7-Azabenzotriazole-1-yl)-N,N,N,N′-tetramethyuronium hexafluorophosphate

Et₃N Triethylamine

MeCN Acetonitrile

HOAc Glacial acetic acid

DMF N,N-Dimethyformamide

EtOAc Ethyl acetate

Et₂O Diethyl ether

Triglyme Tri(ethylene glycol) dimethyl ether

TFA Trifluoroacetic acid

IPE Diisopropyl ether

PhCH₃ Toluene

Pd2(dba)₃ tris(dibezylideneacetone)dipalladium

BINAP rac-2,2′-bis(diphenylphosphino)-1,1′-binaphthyl

Notes:

1) In the case where bromine is present in the molecule the molecular ion reported incorporates the ⁷⁹Br isotope.

2) Unless otherwise stated Flash column chromatography (fcc) was performed using 10 gram packed polypropylene cartridges (Supelco part # 57134A) utilizing a step gradient of DCM:MeOH that contained 0.5% conc. NH_{3(aq)} (eluent-start with DCM then add MeOH, 100:1 [symbol] 50:1 [symbol] 20:1) unless otherwise noted.

3) Preparative Reverse Phase Chromatography-Research samples were purified* using a Gilson preparative chromatography system run by UniPoint® LC System Software installed on a Dell Optiplex GX200 computer (Microsoft Windows NT v.4.00.1381). Unless otherwise stated samples were purified using either a Hewlett Packard CombiHT SB-C18 semi-preparative column (5 μm, 21.2 mm×150 mm; part# 870150-902 KJ1018) or a Modcol C18 preparative column (10 μm, 50.8 mm×250 mm; part# PA000-050025). Flow rates; semi-preparative column (20 mL/min), preparative column (50-80 ml-min). Eluent consisted of a mixture of MeCN/H₂O modified w/0.1% TFA.

A typical sequence comprised:

1) An equilibration (for 3 min at starting gradient concentration)

2) A gradient (started at 40-50% MeCN and ran to 90% MeCN over 7-15 minutes)

3) A flush (for 5 min at 90% MeCN)

*Products were submitted as free bases after purification unless otherwise noted—To remove residual TFA purified products were dissolved in 20% K₂CO_{3(aq)} and extracted with DCM. Organic layer was layer was dried over Na₂SO₄, filtered, and solvent was evaporated under reduced pressure. Products were pumped down under high vacuum for 18 h.

### Example 1

### 1-[5-Methoxy-8-(4-methyl-piperazin-1-yl)-3,4-dihydro-1H-isoquinolin-2-Yl]-2-(4-piperidin-1-ylmethyl-phenyl)-ethanone

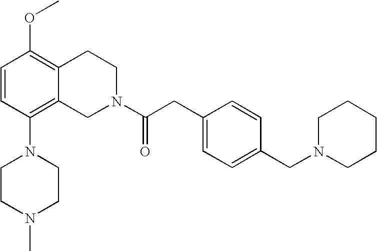

### Example 1a

### 5-methoxyisoquinoline

To a stirred solution of 5-hydroxyisoquinoline (15.01 g, 93.1 mmol) in 200 mL of anhydrous DMF was added sodium tert-butoxide (12.68 g, 137 mmol). The gray-brown mixture turned dark green upon addition of base. When all had dissolved phenyltrimethylammonium chloride was added (23.50 g, 132 mmol), mixture was heated to 153° C. and stirred for 2.5 h. Mixture was cooled to 0° C. and poured into a separatory funnel with 400 mL EtOAc and 500 mL 20% K₂CO_{3(aq)}. After agitation, aqueous layer was removed and extracted in a second separatory funnel containing 300 mL 1:1 EtOAc:Et₂O. Five washings (200 mL each) of 20% K₂CO_{3(aq)} were successively run through each funnel. Organic layers were combined, dried over Na₂SO₄, filtered, and evaporated. Crude product was purified by fcc on silica (eluent—CH₂Cl₂ [symbol] 20:1, CH₂Cl₂:EtOAc [symbol] 10:1, CH₂Cl₂:EtOAc) to give 12.57 g (85%) of a yellow oil. MS m/z 160 (M+1). This procedure is a modification of a method described by Baker, B. R. and McEvoy, F. J. in J. Org. Chem., 1955, 20, 136.

### Example 1b

### 5-Methoxy-8-bromo-isoquinoline

To a stirred solution of 5-methoxyisoquinoline (Example 1a) (11.8 g, 74.1 mmol) in 100 mL HOAc at room temperature was added a solution of bromine (13.6 g (85.4 mmol) dissolved in 50 mL HOAc) over ˜40 min. Dropping rate was adjusted to about 1 mL/min and when addition was complete mixture was stirred overnight. At this time the reaction mixture was slowly poured into a solution of K₂CO₃ (260 g in 1.2 L H₂O) with rapid stirring. After cooling to 0° C. the precipitated orange solid was collected, dissolved in 400 mL DCM, dried over Na₂SO₄, filtered, and evaporated. Crude product was purified by recrystallization from MeCN to give 8.38 g (47% yield, 96% purity) of a tan solid. MS m/z 238 (M+H).

### Example 1c

### 5-Methoxy-8-(-4-methyl-piperizin-1-yl)-isoquinoline

To a flask was added 5-methoxy-8-bromoisoquinoline (Example 1b) (6.72 g, 28.2 mmol), sodium tert-butoxide (3.72 g, 38.7 mmol), BINAP (0.92 g, 1.48 mmol), 110 mL PhCH₃, and 4.6 mL (41.5 mmol) N-methylpiperizine. Mixture was vacuum degassed (3 cycles) with rapid stirring. At this time tris(dibezylideneacetone)dipalladium (0.61 g, 0.67 mmol) was added and mixture was vacuum degassed (3 cycles). Reaction was heated to 112° C. for 18 h, mixed with 20% K₂CO_{3(aq)} (100 mL), and PhCH₃ was evaporated. Residue was extracted with DCM (4×100 mL). Organic layers were combined, dried over Na₂SO₄, filtered, and solvent was evaporated under reduced pressure. Product was purified by fcc on 200 g silica gel to 6.35 g of a dark tan solid. MS: m/z 258 (M+H).

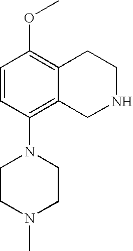

### Example 1d

### 5-Methoxy-8-(-4-methyl-piperizin-1-yl)-1,2,3,4-tetrahydro-isoquinoline

To a stirred solution of 5-methoxy-8-(4-methyl-piperizin-1-yl)-isoquinoline (Example 1c) (5.83 g, 22.7 mmol) in 360 mL of methanol cooled to 0° C. was added NaCNBH₃ (6.20 g, 98.7 mmol). Mixture was stirred for 10 min and then 12 mL (97.2 mmol) BF₃.Et₂O was slowly added (caution H₂ evolution) over 15 min. When addition was complete mixture was stirred for 1 h, ice bath was removed, and mixture was refluxed for 3.5 h. Mixture was cooled to r.t., additional BF₃.Et₂O was added, and reflux was continued for 1 h. Mixture was cooled to r.t., additional BF₃.Et₂O followed by NaCNBH₃ (6.20 g, 98.7 mmol) was added, and reflux was continued for 1 h. Mixture was slowly poured into 300 mL of 20% K₂CO_{3(aq)} and this was stirred for 30 min. Methanol was evaporated and result was extracted with CHCl₃ causing an emulsion to form which was filtered through a bed of diatomaceous earth. Filtrate was extracted with DCM (4×150 mL). Organic layers were combined, dried over Na₂SO₄, filtered, and solvent was evaporated under reduced pressure. Product was purified by fcc on silica to give 320 mg of a tan solid. MS: m/z 262 (M+H).

### Example 1e

### 4-(-1-Morpolinomethyl)phenylacetic acid

4-(Bromomethyl)phenylacetic acid (106 mg, 0.46 mmol) was dissolved in 3 mL of MeCN containing 200 mg (1.45 mmol) anhydrous K₂CO₃. To this was added 250 □L (2.87 mmol) morpholine and mixture was stirred for 3 d. The milky suspension was decanted away from excess K₂CO₃ which was then washed with additional MeCN. MeCN supernatant and washes were combined and evaporated under reduced pressure. Solid was pumped down for 18 h under high vacuum. MS: m/z 236 (M+H). Crude material was used in the next step assuming 0.46 mmol product.

### Example 1

### 1-[5-Methoxy-8-(4-methyl-piperazin-1-yl)-3,4-dihydro-1H-isoquinolin-2-yl]-2-(4-piperidin-1-ylmethyl-phenyl)-ethanone

4-(-1-Morpolinomethyl)phenylacetic acid (Example 1e) (0.46 mmol) and 5-methoxy-8-(-4-methyl-piperizin-1-yl)-1,2,3,4-tetrahydro-isoquinoline (Example 1d) (120 mg, 0.46 mmol) were combined in 5 mL DMF containing 250 □L (1.80 mmol) Et₃N. To this was added 200 mg (0.53 mmol) HATU. Mixture was stirred for 18 h and DMF was evaporated. Result was mixed with 20% K₂CO_{3(aq)} and extracted with DCM (3×20 mL). Organic layer was dried over Na₂SO₄, filtered, and solvent was evaporated under reduced pressure. Product was purified by fcc on silica to give 89 mg of a glass. MS: m/z 479 (M+H).

### Example 2

### 2-(4-Isopropyl-phenyl)-1-(8-(4-methyl-piperazin-1-yl)-3,4-dihydro-1H-isoquinolin-2-yl]-ethanone

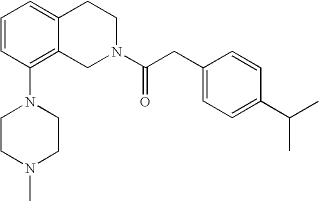

### Example 2a

### 8-Bromoisoquinoline

8-Bromoisoquinoline was prepared by the method described in Organic Reactions Volume VI, pp 200.

### Example 2b

### 8-(4-Methyl-piperazin-1-yl)-isoquinoline

To a flask was added 8-bromoisoquinoline (Example 2a) (480 mg, 2.31 mmol), sodium tert-butoxide (328 mg, 3.41 mmol), BINAP (75 mg, 0.12 mmol), 10 mL PhCH₃, and 380 □L (3.43 mmol) N-methylpiperizine. Mixture was vacuum degassed (3 cycles) with rapid stirring. At this time tris(dibezylideneacetone) dipalladium (52 mg, 0.057 mmol) was added and mixture was vacuum degassed (3 cycles). Reaction was heated to 120° C. for 18 h, mixed with 20% K₂CO_{3(aq)} (50 mL), and extracted with EtOAc (3×50 mL). Organic layers were combined, dried over Na₂SO₄, filtered, and solvent was evaporated under reduced pressure. Product was purified by fcc on silica to give 320 mg of a tan solid. MS: m/z 228 (M+H).

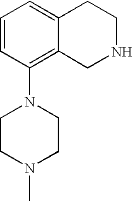

### Example 2c

### 8-(4-Methyl-piperizin-1-yl)-1,2,3,4-tetrahydro-isoquinoline

8-(-4-Methyl-piperizin-1-yl)-isoquinoline (Example 2b) (320 mg, 1.41 mmol) was reduced to 8-(4-methyl-piperizin-1-yl)-1,2,3,4-tetrahydro-isoquinoline using a procedure similar to that described in example 1d. Crude material was purified by fcc on silica to give 330 mg of product. MS: m/z 232 (M+H).

### Example 2

### 2-(4-Isopropyl-phenyl)-1-[8-(4-methyl-piperazin-1-yl)-3,4-dihydro-1H-isoquinolin-2-yl]-ethanone

4-Isopropylphenylacetic acid (96 mg, 0.54 mmol) was reacted with 8-(-4-methyl-piperizin-1-yl)-1,2,3,4-tetrahydro-isoquinoline (Example 2c) (128 mg, 0.55 mmol) using standard HATU coupling conditions (example 1e). Product was purified by fcc on silica to give 162 mg of an oil. MS: m/z 392 (M+H).

### Example 3

### 8-(4-Methyl-piperazin-1-yl)-3,4-dihydro-1H-isoquinoline-2-carboxylic acid (4-morpholin-4-yl-phenyl)-amide

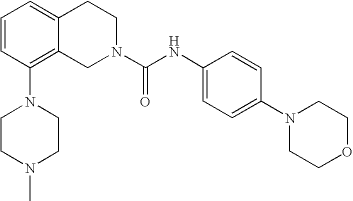

### Example 3

### 8-(4-Methyl-piperazin-1-yl)-3,4-dihydro-1H-isoquinoline-2-carboxylic acid (4-morpholin-4-yl-phenyl)-amide

To a stirred solution of 4-morpholinoaniline (100 mg, 0.56 mmol) in 10 mL DCM was added 1,1′-carbonyldiimidazole (91 mg, 0.56 mmol). Mixture was stirred for 3 h, then 8-(4-methyl-piperizin-1-yl)-1,2,3,4-tertrahydro-isoquinoline (Example 2c) (140 mg, 0.61 mmol) was added and stirring was continued for 18 h. Mixture was then diluted with DCM (40 mL) and this was extracted with 20% K₂CO₃ (2×15 mL). Organic layer was dried over Na₂SO₄, filtered, and evaporated. Product was purified by fcc on silica to give 68 mg of a foam. MS: m/z 436 (M+H).

### Example 4

### 5-Methoxy-8-phenyl-3,4-dihydro-1H-isoquinoline-2-carboxylic acid (4-morpholin-4-yl-phenyl)-amide

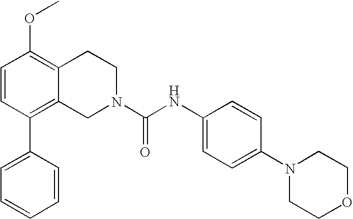

### Example 4b

### 5-Methoxy-8-phenyl-isoquinoline

To a flask was added 5-methoxy-8-bromoisoquinoline (Example 1b) (244 mg, 1.02 mmol), benzeneboronic acid (119 mg, 0.98 mmol), triphenylphosphine (17 mg, 0.065 mmol), 3 mL EtOH, and potassium carbonate (150 mg, 1.42 mmol) dissolved in 1 mL H₂O. Mixture was vacuum degassed (3 cycles) with rapid stirring. At this time palladium acetate (8 mg, 0.04 mmol) was added and mixture was vacuum degassed (3 cycles). Reaction was heated to 90° C. for 18 h, mixed with 20% K₂CO_{3(aq)} (25 mL), and extracted with DCM (3×20 mL). Organic layers were combined, dried over Na₂SO₄, filtered, and solvent was evaporated under reduced pressure. Crude material was purified by fcc on silica to give 230 mg of product. MS: m/z 236 (M+H).

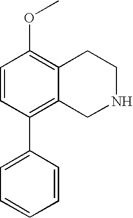

### Example 4a

### 5-Methoxy-8-phenyl-1,2,3,4-tetrahydro-isoquinoline

To a stirred solution of 5-methoxy-8-phenyl-isoquinoline (Example 4b) (230 mg, 0.98 mmol) in 30 mL of methanol cooled to 0° C. was added NaCNBH₃ (307 mg, 4.88 mmol). Mixture was stirred for 10 min and then 600 □L (4.80 mmol) BF₃.Et₂O was added (caution H₂ evolution). When addition was complete mixture was stirred for 1 h, ice bath was removed, and mixture was refluxed for 3.5 h. At this time reaction was mixed with 20% K₂CO_{3(aq)} (20 mL), methanol was evaporated, and result was extracted with DCM (3×20 mL). Organic layers were combined, dried over Na₂SO₄, filtered, and solvent was evaporated under reduced pressure. Product was purified by fcc on silica to give 230 mg of a white solid. MS: m/z 240 (M+H).

### Example 4

### 5-Methoxy-8-phenyl-3,4-dihydro-1H-isoquinoline-2-carboxylic acid (4-morpholin-4-yl-phenyl)-amide

5-Methoxy-8-phenyl-1,2,3,4-tetrahydro-isoquinoline was reacted with 4-morpholinoaniline (100 mg, 0.56 mmol and 1,1′-carbonyldiimidazole (91 mg, 0.56 mmol) using a standard method described in example 3, synthesis of 1. Product was purified by fcc on silica to give 109 mg of a white solid. MS: m/z 444 (M+H).

### Example 5

### 1-[5-Benzyloxy-8-(4-methyl-piperazin-1-yl)-3,4-dihydro-1H-isoquinolin-2-yl]-2-(4-isopropyl-phenyl)-ethanone

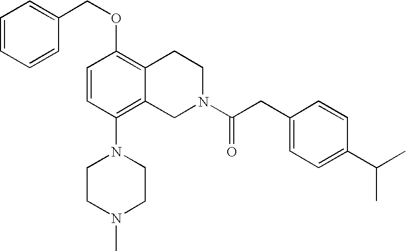

### Example 5a

### 5-Benzyloxyisoquinoline

The procedure for benzylation of 5-hydroxyisoquinoline has been published in Bioorg. Med. Chem. 1999, 7, 2647-2666.

### Example 5b

### 5-Benzyloxy-8-bromo-isoquinoline

To a stirred solution of 5-benzyloxyisoquinoline (Example 5a) (2.12 g, 9.01 mmol) and NaOAc (1.54 g, 18.8 mmol) in 30 mL HOAc at room temperature was added a solution of bromine (500 □L, 9.76 mmol) dissolved in 9 mL HOAc) over ˜10 min. When addition was complete mixture was stirred overnight, 20 mL 20% K₂CO_{3(aq)} was slowly added, and HOAc was evaporated under reduced pressure. Result was mixed with 20 mL 20% K₂CO_{3(aq)} and extracted with EtOAc. Extracts were combined, dried over Na₂SO₄, filtered, and evaporated. Crude product was purified by fcc on silica (eluent—CH₂Cl₂ [symbol] 10:1, CH₂Cl₂:EtOAc [symbol] 5:1, CH₂Cl₂:EtOAc, CH₂Cl₂:EtOAc [symbol] 1:1) to give 1.05 g of an oil. MS m/z 314 (M+H).

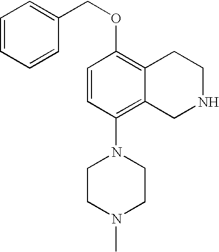

### Example 5c

### 5-Benzyloxy-8-(-4-methyl-piperizin-1-yl)-isoquinoline

5-Benzyloxy-8-bromo-isoquinoline (Example 5b) (1.05 g, 3.34 mmol) was coupled with N-methylpiperizine using conditions similar to that described in example 2b. Product was purified by fcc on silica to give 760 mg of a light gray solid. MS: m/z 334 (M+H).

### Example 5d

### 5-Benzyloxy-8-(4-methyl-piperizin-1-yl)-1,2,3,4-tetrahydro-isoquinoline

5-Benzyloxy-8-(-4-methyl-piperizin-1-yl)-isoquinoline (Example 5c) (250 mg, 0.75 mmol) was reduced to 5-benzyloxy-8-(-4-methyl-piperizin-1-yl)-1,2,3,4-tetrahydro-isoquinoline using a procedure similar to that described in example 1d. Product was purified by fcc on silica to give 230 mg of a light gray solid. MS: m/z 334 (M+H).

### Example 5

### 1-[5-Benzyloxy-8-(4-methyl-piperazin-1-yl)-3,4-dihydro-1H-isoquinolin-2-yl]-2-(4-isopropyl-phenyl)-ethanone

4-Isopropylphenylacetic acid (121 mg, 0.68 mmol) was reacted with 5-benzyloxy-8-(4-methyl-piperizin-1-yl)-1,2,3,4-tetrahydro-isoquinoline (Example 5d) (230 mg, 0.68 mmol) using standard HATU coupling conditions (example 1). Product was purified by fcc on silica to give 320 mg of an oil. MS: m/z 498 (M+H).

### Example 6

### 1-[5-Hydroxy-8-(4-methyl-piperazin-1-yl)-3,4-dihydro-1H-isoquinolin-2-yl]-2-(4-isopropyl-phenyl)-ethanone

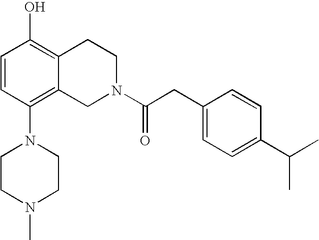

### Example 6

### 1-[5-Hydroxy-8-(4-methyl-piperazin-1-yl)-3,4-dihydro-1H-isoquinolin-2-yl]-2-(4-isopropyl-phenyl)-ethanone

To a Parr® hydrogenation flask was added 55 mg 10% Pd/C followed by 40 mL absolute EtOH. To this was added 1-[5-benzyloxy-8-(4-methyl-piperazin-1-yl)-3,4-dihydro-1H-isoquinolin-2-yl]-2-(4-isopropyl-phenyl)-ethanone (Example 5) (208 mg, 0.42 mmol) and 500 □L HOAc. Mixture was shaken under H₂ atmosphere (50 psi) for 18 h, filtered through diatomaceous earth, and solvent was evaporated. Product was purified by fcc on silica to give 98 mg of an oil. MS: m/z 408 (M+H).

### Example 7

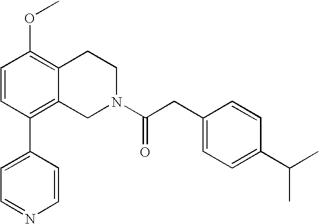

### Example 7a

### 5-Methoxy-8-bromo-1,2,3,4-tetrahydro-isoquinoline

5-Methoxy-8-bromo-isoquinoline (2.41 g, 10.1 mmol) was reduced to 5-methoxy-8-bromo-1,2,3,4-tetrahydro-isoquinoline using a procedure similar to that described in example 4b. Product was purified by fcc on silica to give 2.19 g of a brown solid. MS: m/z 242 (M+H).

### Example 7b

### 1-(8-Bromo-5-methoxy-3,4-dihydro-1H-isoquinolin-2-yl)-2-(4-isopropyl-phenyl)-ethanone

4-Isopropylphenylacetic acid (684 mg, 3.84 mmol) was reacted with 5-methoxy-8-bromo-1,2,3,4-tetrahydro-isoquinoline (930 mg, 3.84 mmol) using standard HATU coupling conditions (example 1). Product was purified by fcc on silica to give 1.42 g of an orange solid. MS: m/z 402 (M+H).

### Example 7

### 2-(4-Isopropyl-phenyl)-1-(5-methoxy-8-pyridin-4-yl-3,4-dihydro-1H-isoquinolin-2-yl)-ethanone

1-(8-Bromo-5-methoxy-3,4-dihydro-1H-isoquinolin-2-yl)-2-(4-isopropyl-phenyl)-ethanone (400 mg, 0.99 mmol) was reacted with pyridine-4-boronic acid (130 mg, 1.06 mmol) using coupling conditions similar to that described in example 4a. Product was purified by fcc on silica to give 155 mg of an off-white foam. MS: m/z 401 (M+).

### Example 8

### 2-(4-Isopropyl-phenyl)-1-[5-methoxy-8-(1-methyl-1,2,3,6-tetrahydro-pyridin-4-yl)-3,4-dihydro-1H-isoquinolin-2-yl]-ethanone

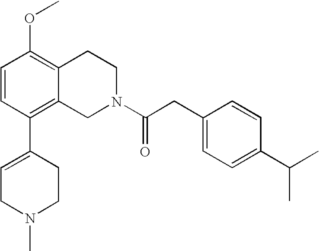

### Example 8a

### 5-Methoxy-8-bromo-3,4-dihydro-1H-isoquinoline-2-carboxylic acid tert-butyl ester

To a stirred solution of 5-methoxy-8-bromo-1,2,3,4-tetrahydro-isoquinoline (297 mg, 1.23 mmol) and Et₃N (320 □L, 2.30 mmol) in 10 mL of DCM was added di-tert-butyl dicarbonate (272 mg, 1.24 mmol). Mixture was stirred for [8 h, diluted with DCM (40 mL), and extracted with 20% K₂CO_{3(aq)} (2×20 mL). Organic layer was dried over Na₂SO₄, filtered, and solvent was evaporated under reduced pressure. Product was purified by fcc on silica (CH₂Cl₂:EtOAc 20:1 [symbol] 10:1) to give 230 mg of a white solid. MS: m/z 283 (M-59).

### Example 8b

### 5-Methoxy-8-pyridin-4-yl-3,4-dihydro-1H-isoquinoline-2-carboxylic acid tert-butyl ester

5-Methoxy-8-bromo-3,4-dihydro-1H-isoquinoline-2-carboxylic acid tert-butyl ester (Example 8a) (410 mg, 1.20 mmol) was reacted with pyridine-4-boronic acid (150 mg, 1.22 mmol) using coupling conditions similar to that described in example 4a. Crude material was purified by fcc on silica to give 254 mg of product. MS: m/z 341 (M+H).

### Example 8c

### 4-(2-tert-Butoxycarbonyl-5-methoxy-1,2,3,4-tetrahydro-isoquinolin-8-yl)-1-methyl-pyridinium iodide

5-Methoxy-8-pyridin-4-yl-3,4-dihydro-1H-isoquinoline-2-carboxylic acid tert-butyl ester (Example 8b) (0.58 g, 1.7 mmol) was dissolved in 10 mL MeCN containing a few pieces of copper wire (18 ga). To this was added methyl iodide (1.0 mL, 3.1 mmol). Flask was tightly stoppered, wrapped in aluminum foil, and stirred for 18 h. The resulting brown solution was filtered to remove copper wire and solvent was evaporated to give 0.82 g of product. MS: m/z 355 (M−100).

### Example 8d

### 4-(5-Methoxy-1,2,3,4-tetrahydro-isoquinolin-8-yl)-1-methyl-pyridinium iodide hydrochloride

4-(2-tert-Butoxycarbonyl-5-methoxy-1,2,3,4-tetrahydro-isoquinolin-8-yl)-1-methyl-pyridinium iodide (Example 8c) (0.78 g, 1.62 mmol) was dissolved in 30 mL DCM and to this was added 60 mL HCl solution (2.0 M HCl in Et₂O). Mixture was stirred for 1.5 h and filtered. Filter cake was washed with Et₂O (3×30 mL) and dried under high vacuum for 6 h to give 0.61 g of a light brown solid. MS: m/z 255 (M+).

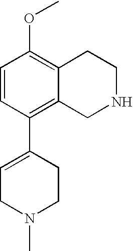

### Example 8e

### 5-Methoxy-8-(1-methyl-1,2,3,6-tetrahydro-pyridin-4-yl)-1,2,3,4-tetrahydro-isoquinoline

To a stirred solution of 4-(5-methoxy-1,2,3,4-tetrahydro-isoquinolin-8-yl)-1-methyl-pyridinium iodide hydrochloride (Example 8d) (313 mg, 0.75 mmol) in 10 mL of methanol cooled to 0° C. was added NaCNBH₃ (0.60 g, 9.5 mmol). Mixture was stirred for 10 min and then 0.9 mL (7.3 mmol) BF₃.Et₂O was slowly added (caution H₂ evolution). When addition was complete mixture was stirred for 1 h, ice bath was removed, and mixture was refluxed for 3.5 h. Mixture was cooled to r.t., additional BF₃.Et₂O (0.9 mL, 7.3 mmol) and NaCNBH₃ (0.60 g, 9.5 mmol) was added, and reflux was continued for 3 h. Mixture was cooled to r.t., additional BF₃.Et₂O (0.7 mL, 5.7 mmol) and NaCNBH₃ (0.40 g, 6.4 mmol) was added, and reflux was continued for 18 h. Mixture was slowly poured into 20 mL of 20% K₂CO_{3(aq)} and this was stirred for 10 min. Methanol was evaporated and result was extracted with DCM (3×20 mL). Organic layers were combined, dried over Na₂SO₄, filtered, and solvent was evaporated under reduced pressure. Product was purified by fcc on silica to give 78 mg of a tan solid. MS: m/z 259 (M+).

### Example 8

### 2-(4-Isopropyl-phenyl)-1-[5-methoxy-8-(1-methyl-1,2,3,6-tetrahydro-pyridin-4-yl)-3,4-dihydro-1H-isoquinolin-2-yl]-ethanone

4-Isopropylphenylacetic acid (54 mg, 0.30 mmol) and 5-methoxy-8-(1-methyl-1,2,3,6-tetrahydro-pyridin-4-yl)-1,2,3,4-tetrahydro-isoquinoline (Example 8e) (78 mg, 0.30 mmol) were combined in 10 mL DCM containing 130 □L (0.93 mmol) Et₃N. To this was added 120 mg (0.32 mmol) HATU. Mixture was stirred for 18 h, diluted with DCM (30 mL), and extracted with 20% K₂CO_{3(aq)}. Organic layer was dried over Na₂SO₄, filtered, and solvent was evaporated under reduced pressure. Product was purified by fcc on silica to give 68 mg of an oil. MS: m/z 419 (M+H).

### Example 9

### 4-{2-[5-Methoxy-8-(4-methyl-piperazin-1-yl)-6-propyl-3,4-dihydro-1H-isoquinolin-2-yl]-2-oxo-ethyl}-N-propyl-benzenesulfonamide

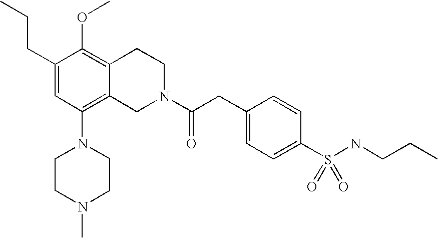

### Example 9a

### 5-Allyloxy-isoquinoline

5-Allyloxy-isoquinoline was prepared utilizing a procedure from Bioorg. Med. Chem. 1999, 7, 2647-2666 which describes the benzylation of 5-hydroxyisoquinoline (example 5a). In this example allyl bromide was substituted for benzyl bromide. ¹H NMR (CDCl₃) δ 9.21 (s, 1H), 8.53 (d, 1H), 8.05 (d, 1H), 7.55 (d, 1H), 7.49 (t, 1H), 7.00 (d, 1H), 6.16 (ddt, 1H), 5.52 (dd, 1H), 5.37 (dd, 1H), 4.80-4.70 (m, 2H).

### Example 9b

### 6-Allyl-isoquinolin-5-ol

A solution of 5-allyloxy-isoquinoline (Example 9a) (1.47 g, 7.96 mmol) in 10 mL triglyme was heated to 180° C. for 4 h. Solvent was distilled off under reduced pressure (50 mmHg, 80° C.), solid residue was dissolved in 60 mL Et₂O, and solvent was evaporated under reduced pressure. Tan-yellow solid was pumped down under high vacuum for 18 h to give 1.25 g of product. ¹H NMR (CDCl₃) δ 9.55 (s, 1H), 9.18 (s, 1H), 8.44 (d, 1H), 8.02 (d, 1H), 7.57 (d, 1H), 7.42 (d, 1H), 6.00 (ddt, 1H), 5.09 (dm, 1H), 5.04 (d, 1H), 3.57 (dm, 2H).

### Example 9c

### 6-Propyl-isoquinolin-5-ol

To a Parr® hydrogenation flask was added 20 mg 10% Pd/C followed by 20 mL absolute EtOH. To this was added 6-allyl-isoquinolin-5-ol (Example 9b) (530 mg, 2.81 mmol) and 500 □L HOAc. Mixture was shaken under H₂ atmosphere (48 psi, 55° C.) for 18 h, filtered through diatomaceous earth, and solvent was evaporated. This gave 450 mg crude product. MS: m/z 188 (M+H).

### Example 9d

### 8-Bromo-6-propyl-isoquinolin-5-ol

To a stirred solution of 6-propyl-isoquinolin-5-ol (Example 9c) (450 mg, 2.41 mmol) in 3 mL HOAc at room temperature was added a solution of bromine (130 □L (2.53 mmol) dissolved in 1 mL HOAc). Mixture was stirred overnight, quenched by the addition of 15 mL saturated NaHCO_{3(aq)} and 15 mL H₂O. The resulting precipitate was collected by filtration, washed with H₂O (2 mL), and pumped down under high vacuum to give 510 mg of product. MS: m/z 266 (M+H).

### Example 9e

### 8-Bromo-6-propyl-1,2,3,4-tetrahydro-isoquinolin-5-ol

To a stirred solution of 8-bromo-6-propyl-isoquinolin-5-ol (Example 9d) (510 mg, 1.92 mmol) in 25 mL of methanol cooled to 0° C. was added NaCNBH₃ (350 mg, 5.57 mmol). Mixture was stirred for 10 min and then 700 □L (5.67 mmol) BF₃.Et₂O was slowly added (caution H₂ evolution). When addition was complete mixture was stirred for 1 h, ice bath was removed, and mixture was refluxed for 3.5 h. Methanol was evaporated, residue was mixed with 20 mL saturated NaHCO_{3(aq)}/H₂O (1:1). Result was extracted with CHCl₃ (3×20 mL), organic layers were combined, dried over Na₂SO₄, filtered, and solvent was evaporated under reduced pressure. Residue was pumped down under high vacuum to give 450 mg of crude product. MS: m/z 270 (M+H).

### Example 9f

### 8-Bromo-5-hydroxy-6-propyl-3,4-dihydro-1H-isoquinoline-2-carboxylic acid tert-butyl ester

8-Bromo-6-propyl-1,2,3,4-tetrahydro-isoquinolin-5-ol (Example 9e) (450 mg, 1.67 mmol) was reacted with di-tert-butyl dicarbonate (730 mg, 3.34 mmol) using conditions similar to that described in example 8a. Product was purified by fcc on silica (CH₂C₂:EtOAc, 20:1 [symbol] 10:1) to give 340 mg of a white solid. MS: m/z 270 (M−100).

### Example 9g

### 8-Bromo-5-methoxy-6-propyl-3,4-dihydro-1H-isoquinoline-2-carboxylic acid tert-butyl ester

8-Bromo-5-hydroxy-6-propyl-3,4-dihydro-1H-isoquinoline-2-carboxylic acid tert-butyl ester (Example 9f) (340 mg, 0.91 mmol) was dissolved in 7 mL DMF containing anhydrous K₂CO₃ (640 mg, 4.63 mmol). To this was added methyl iodide (300 □L, 4.81 mmol). Flask was tightly stoppered, wrapped in aluminum foil, and stirred for 2 d. Reaction was diluted with 60 mL 1:1, Et₂O:EtOAc and extracted with H₂O (4×20 mL). Organic layer was dried over Na₂SO₄, filtered, and solvent was evaporated under reduced pressure. Product was purified by fcc on silica (Hexane:CH₂Cl₂:EtOAc, 55:45:5) to give 272 mg of a clear oil. MS: m/z 325 (M-59).

### Example 9h

### 5-Methoxy-8-(4-methyl-piperazin-1-yl)-6-propyl-3,4-dihydro-1H-isoquinoline-2-carboxylic acid tert-butyl ester

8-Bromo-5-methoxy-6-propyl-3,4-dihydro-1H-isoquinoline-2-carboxylic acid tert-butyl ester (Example 9g) (272 mg, 0.71 mmol) was coupled with N-methylpiperizine using conditions similar to that described in example 2b. For this reaction Cs₂CO₃ was used in place of sodium tert-butoxide. Crude material was purified by fcc on silica to give 120 mg of product. MS: m/z 404 (M+H).

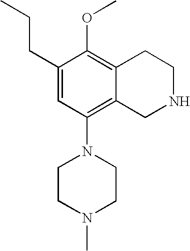

### Example 9i

### 5-Methoxy-8-(4-methyl-piperazin-1-yl)-6-propyl-1,2,3,4-tetrahydro-isoquinoline

5-Methoxy-8-(4-methyl-piperazin-1-yl)-6-propyl-3,4-dihydro-1H-isoquinoline-2-carboxylic acid tert-butyl ester (Example 9h) was dissolved in 15 mL DCM and to this was added 2 mL TFA. Mixture was stirred for 2 h and solvent/TFA was evaporated under reduced pressure. Residue was mixed with 15 mL 20% KOH_{(aq)} and extracted with CHCl₃ (4×20 mL). Extracts were combined, dried over Na₂SO₄, filtered, and evaporated under reduced pressure. Crude material was purified by fcc on silica to give 60 mg of product. MS: m/z 304 (M+H).

### Example 9

### 4-{2-[5-Methoxy-8-(4-methyl-piperazin-1-yl)-6-propyl-3,4-dihydro-1H-isoquinolin-2-yl]-2-oxo-ethyl}-N-propyl-benzenesulfonamide

(4-Propylsulfamoyl-phenyl)-acetic acid (47 mg, 0.18 mmol) was reacted with 5-methoxy-8-(4-methyl-piperazin-1-yl)-6-propyl-1,2,3,4-tetrahydro-isoquinoline (Example 9i) (55 mg, 0.18 mmol) using standard HATU coupling conditions (example 8). Product was purified by fcc on silica to give 53 mg of an off-white foam. MS: m/z 543 (M+H).

### Example 10

### 4-{2-[5-Methoxy-8-(4-methyl-piperazin-1-yl)-3,4-dihydro-1H-isoquinolin-2-yl]-2-oxo-ethyl}-N-propyl-benzenesulfonamide

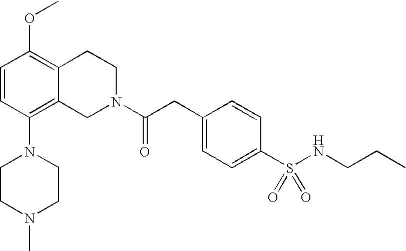

### Example 10a

### (4-Chlorosulfonyl-phenyl)-acetic acid

Prepared by the method of Hornby, R. and Cremlyn, R. J. in J. Chem. Soc. C, 1969, 1341-1345.

### Example 10b

### (4-Propylsulfamoyl-phenyl)-acetic acid

A solution of N-propylamine (500 □L, 6.1 mmol) in 5 mL methanol was added to solid (4-chlorosulfonyl-phenyl)-acetic acid (Example 10a) (120 mg, 0.51). The mixture was stirred at r.t for 3 h, methanol was evaporated, reside was dissolved in 20 mL DCM, and extracted with 1N HCl (4×8 mL). Organic layer was dried over-Na₂SO₄, filtered, and evaporated under reduced pressure to give 147 mg of product. MS: m/z 258 (M+H).

### Example 10

### 4-{2-[5-Methoxy-8-(4-methyl-piperazin-1-yl)-3,4-dihydro-1H-isoquinolin-2-yl]-2-oxo-ethyl}-N-propyl-benzenesulfonamide

(4-Propylsulfamoyl-phenyl)-acetic acid (Example 10b) (147 mg, 0.57 mmol) was reacted with 5-methoxy-8-(-4-methyl-piperizin-1-yl)-1,2,3,4-tetrahydro-isoquinoline (150 mg, 0.57 mmol) using standard HATU coupling conditions (example 8). Product was purified by fcc on silica to give 102 mg of an off-white foam. MS: m/z 501 (M+H).

### Example 11

### N-Isopropyl-4-{2-[5-methoxy-8-(4-methyl-piperazin-1-yl)-3,4-dihydro-1H-isoquinolin-2-yl]-2-oxo-ethyl}-benzenesulfonamide

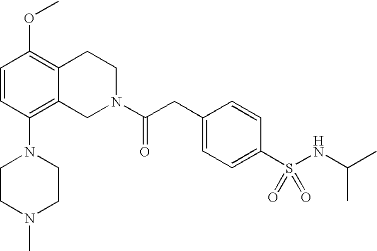

### Example 11a

### (4-Isopropylsulfamoyl-phenyl)-acetic acid

Isopropylamine (500 □L, 5.9 mmol) was reacted with (4-chlorosulfonyl-phenyl)-acetic acid (120 mg, 0.51) to give 101 mg of product using a procedure like that described in example 10b. MS: m/z 258 (M+H).

### Example 11

### N-Isopropyl-4-{2-[5-methoxy-8-(4-methyl-piperazin-1-yl)-3,4-dihydro-1H-isoquinolin-2-yl]-2-oxo-ethyl}-benzenesulfonamide

(4-Isopropylsulfamoyl-phenyl)-acetic acid (101 mg, 0.39 mmol) was reacted with 5-methoxy-8-(4-methyl-piperizin-1-yl)-1,2,3,4-tetrahydro-isoquinoline (103 mg, 0.39 mmol) using standard HATU-coupling conditions (example 8). Product was purified by fcc on silica to give 160 mg of an off-white foam. MS: m/z 501 (M+H).

### Example 12

### N-tert-Butyl-4-{2-[5-methoxy-8-(4-methyl-piperazin-1-yl)-3,4-dihydro-1H-isoquinolin-2-yl]-2-oxo-ethyl}-benzenesulfonamide

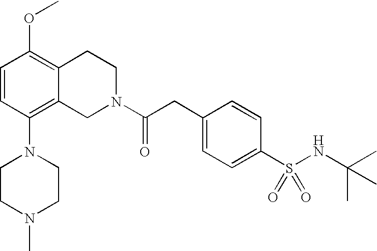

### Example 12a

### (4-tert-Butylsulfamoyl-phenyl)-acetic acid

tert-Butylamine (500 □L, 4.8 mmol) was reacted with (4-chlorosulfonyl-phenyl)-acetic acid (120 mg, 0.51) to give 46 mg of product using a procedure like that described in example 10b. MS: m/z 257 (M−15+H).

### Example 12

### N-tert-Butyl-4-{2-[5-methoxy-8-(4-methyl-piperazin-1-yl)-3,4-dihydro-1H-isoquinolin-2-yl]-2-oxo-ethyl}-benzenesulfonamide

(4-tert-Butylsulfamoyl-phenyl)-acetic acid (46 mg, 0.17 mmol) was reacted with 5-methoxy-8-(4-methyl-piperizin-1-yl)-1,2,3,4-tetrahydro-isoquinoline (44 mg, 0.17 mmol) using standard HATU coupling conditions (example 8). Product was purified by fcc on silica to give 66 mg of an off-white foam. MS: m/z 515 (M+H).

### Example 13

### N-Benzyl-4-{2-[5-methoxy-8-(4-methyl-piperazin-1-yl)-3,4-dihydro-1H-isoquinolin-2-yl]-2-oxo-ethyl}-benzenesulfonamide

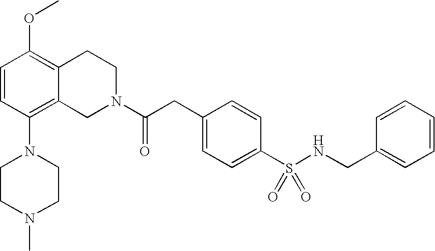

### Example 13a

### (4-Benzylsulfamoyl-phenyl)-acetic acid

Benzylamine (600 □L, 6.4 mmol) was reacted with (4-chlorosulfonyl-phenyl)-acetic acid (120 mg, 0.51) to give 96 mg of product using a procedure like that described in example 10b. MS: m/z 306 (M+H).

### Example 13

### N-Benzyl-4-{2-[5-methoxy-8-(4-methyl-piperazin-1-yl)-3,4-dihydro-1H-isoquinolin-2-yl]-2-oxo-ethyl}-benzenesulfonamide

(4-Benzylsulfamoyl-phenyl)-acetic acid (96 mg, 0.33 mmol) was reacted with 5-methoxy-8-(-4-methyl-piperizin-1-yl)-1,2,3,4-tetrahydro-isoquinoline (85 mg, 0.33 mmol) using standard HATU coupling conditions (example 8). Product was purified by fcc on silica to give 134 mg of an off-white foam. MS: m/z 549 (M+H).

### Example 14

### N-(2-Methoxy-benzyl)-4-{2-[5-methoxy-8-(4-methyl-piperazin-1-yl)-3,4-dihydro-1H-isoquinolin-2-yl]-2-oxo-ethyl}-benzenesulfonamide

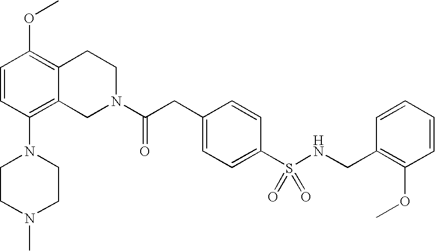

### Example 14a

### [4-(2-Methoxy-benzylsulfamoyl)-phenyl]-acetic acid

2-Methoxybenzylamine (800 □L, 6.2 mmol) was reacted with (4-chlorosulfonyl-phenyl)-acetic acid (120 mg, 0.51) to give 101 mg of product (after purification by fcc on silica) using a procedure like that described in example 10b. MS: m/z 358 (M+23).

### Example 14

### N-(2-Methoxy-benzyl)-4-{2-[5-methoxy-8-(4-methyl-piperazin-1-yl)-3,4-dihydro-1H-isoquinolin-2-yl]-2-oxo-ethyl}-benzenesulfonamide

[4-(2-Methoxy-benzylsulfamoyl)-phenyl]-acetic acid (101 mg, 0.30 mmol) was reacted with 5-methoxy-8-(4-methyl-piperizin-1-yl)-1,2,3,4-tetrahydro-isoquinoline (80 mg, 0.31 mmol) using standard HATU coupling conditions (example 8). Product was purified by fcc on silica to give 139 mg of an off-white foam. MS: m/z 579 (M+H).

### Example 15

### N-(3-Methoxy-benzyl)₄-{2-[5-methoxy-8-(4-methyl-piperazin-1-yl)-3,4-dihydro-1H-isoquinolin-2-yl]-2-oxo-ethyl}-benzenesulfonamide

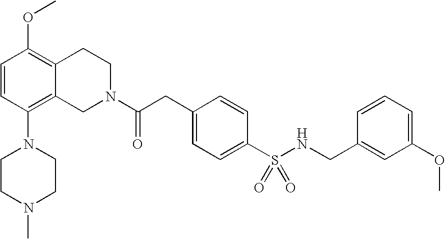

### Example 15a

### [4-(3-Methoxy-benzylsulfamoyl)-phenyl]-acetic acid

3-Methoxybenzylamine (800 □L, 6.2 mmol) was reacted with (4-chlorosulfonyl-phenyl)-acetic acid (120 mg, 0.51) to give 157 mg of product (after purification by fcc on silica) using a procedure like that described in example 10b. MS: m/z 336 (M+H).

### Example 15

### N-(3-Methoxy-benzyl)₄-{2-[5-methoxy-8-(4-methyl-piperazin-1-yl)-3,4-dihydro-1H-isoquinolin-2-yl]-2-oxo-ethyl}-benzenesulfonamide

(4-(3-Methoxy-benzylsulfamoyl)-phenyl]-acetic acid (157 mg, 0.47 mmol) was reacted with 5-methoxy-8-(4-methyl-piperizin-1-yl)-1,2,3,4-tetrahydro-isoquinoline (122 mg, 0.47 mmol) using standard HATU coupling conditions (example 8). Product was purified by fcc on silica to give 248 mg of an off-white foam. MS: m/z 579 (M+H).

### Example 16

### N-(4-Methoxy-benzyl)₄-{2-[5-methoxy-8-(4-methyl-piperazin-1-yl)-3,4-dihydro-1H-isoquinolin-2-yl]-2-oxo-ethyl}-benzenesulfonamide

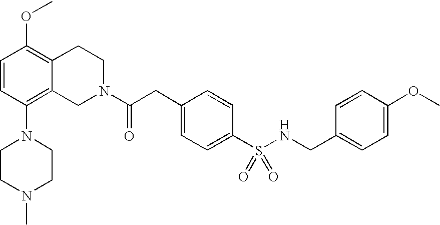

### Example 16a

### [4-(4-Methoxy-benzylsulfamoyl)-phenyl]-acetic acid

4-Methoxybenzylamine (800 □L, 6.2 mmol) was reacted with (4-chlorosulfonyl-phenyl)-acetic acid (120 mg, 0.51) to give 129 mg of product (after purification by fcc on silica) using a procedure like that described in example 10b. MS: m/z 358 (M+23).

### Example 16

### N-(4-Methoxy-benzyl)-4-{2-[5-methoxy-8-(4-methyl-piperazin-1-yl)-3,4-dihydro-1H-isoquinolin-2-yl]-2-oxo-ethyl}-benzenesulfonamide

[4-(4-Methoxy-benzylsulfamoyl)-phenyl]-acetic acid (129 mg, 0.38 mmol) was reacted with 5-methoxy-8-(4-methyl-piperizin-1-yl)-1,2,3,4-tetrahydro-isoquinoline (100 mg, 0.38 mmol) using standard HATU coupling conditions (example 8). Product was purified by fcc on silica to give 186 mg of an off-white foam. MS: m/z 579 (M+H).

### Example 17

### 4-{2-[5-Methoxy-8-(4-methyl-piperazin-1-yl)-3,4-dihydro-1H-isoquinolin-2-yl]-2-oxo-ethyl}-H-(2-methoxy-phenyl)-benzenesulfonamide

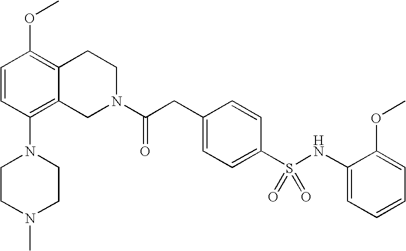

### Example 17a

### [4-(2-Methoxy-phenylsulfamoyl)-phenyl]-acetic acid

o-Anisidine (800 □L, 7.09 mmol) was reacted with (4-chlorosulfonyl-phenyl)-acetic acid (240 mg, 1.02) to give 123 mg of product (after purification by fcc on silica) using a procedure like that described in example 10b. MS: m/z 322 (M+H).

### Example 17

### 4-{2-[5-Methoxy-8-(4-methyl-piperazin-1-yl)-3,4-dihydro-1H-isoquinolin-2-yl]-2-oxo-ethyl}-H-(2-methoxy-phenyl)-benzenesulfonamide

[4-(2-Methoxy-phenylsulfamoyl)-phenyl]-acetic acid (123 mg, 0.38 mmol) was reacted with 5-methoxy-8-(4-methyl-piperizin-1-yl)-1,2,3,4-tetrahydro-isoquinoline (108 mg, 0.41 mmol) using standard HATU coupling conditions (example 8). Product was purified by fcc on silica to give 214 mg of an off-white foam. MS: m/z 565 (M+H).

### Example 18

### 4-{2-[5-Methoxy-8-(4-methyl-piperazin-1-yl)-3,4-dihydro-1H-isoquinolin-2-yl]-2-oxo-ethyl}-H-(3-methoxy-phenyl)-benzenesulfonamide

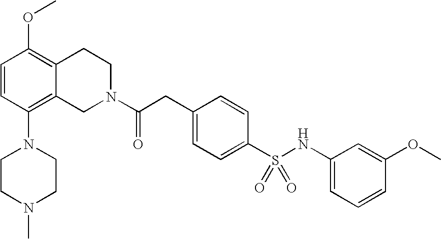

### Example 18a

### [4-(3-Methoxy-phenylsulfamoyl)-phenyl]-acetic acid

m-Anisidine (800 □L, 7.09 mmol) was reacted with (4-chlorosulfonyl-phenyl)-acetic acid (240 mg, 1.02) to give 101 mg of product (after purification by fcc on silica) using a procedure like that described in example 10b. MS: m/z 365 (M+41).

### Example 18

### 4-{2-[5-Methoxy-8-(4-methyl-piperazin-1-yl)-3,4-dihydro-1H-isoquinolin-2-yl]-2-oxo-ethyl}-H-(3-methoxy-phenyl)-benzenesulfonamide

[4-(3-Methoxy-phenylsulfamoyl)-phenyl]-acetic acid (101 mg, 0.31 mmol) was reacted with 5-methoxy-8-(4-methyl-piperizin-1-yl)-1,2,3,4-tetrahydro-isoquinoline (82 mg, 0.31 mmol) using standard HATU coupling conditions (example 8). Product was purified by fcc on silica to give 140 mg of an off-white foam. MS: m/z 565 (M+H).

### Example 19

### 4-{2-[5-Methoxy-8-(4-methyl-piperazin-1-yl)-3,4-dihydro-1H-isoquinolin-2-yl]-2-oxo-ethyl}-H-(4-methoxy-phenyl)-benzenesulfonamide

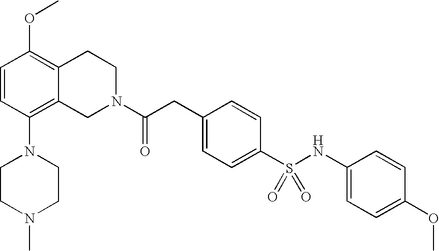

### Example 19a

### [4-(4-Methoxy-phenylsulfamoyl)-phenyl]-acetic acid

p-Anisidine (800 □L, 7.09 mmol) was reacted with (4-chlorosulfonyl-phenyl)-acetic acid (240 mg, 1.02) to give 118 mg of product (after purification by fcc on silica) using a procedure like that described in example 10b. MS: m/z 322 (M+H).

### Example 19

### 4-{2-[5-Methoxy-8-(4-methyl-piperazin-1-yl)-3,4-dihydro-1H-isoquinolin-2-yl]-2-oxo-ethyl}-H-(4-methoxy-phenyl)-benzenesulfonamide

[4-(4-Methoxy-phenylsulfamoyl)-phenyl]-acetic acid (118 mg, 0.37 mmol) was reacted with 5-methoxy-8-(-4-methyl-piperizin-1-yl)-1,2,3,4-tetrahydro-isoquinoline (100 mg, 0.38 mmol) using standard HATU coupling conditions (example 8). Product was purified by fcc on silica to give 205 mg of an off-white foam. MS: m/z 565 (M+H).

### Example 20

### N-Cyclopropyl-4-{2-[5-methoxy-8-(4-methyl-piperazin-1-yl)-3,4-dihydro-1H-isoquinolin-2-yl]-2-oxo-ethyl}-benzenesulfonamide

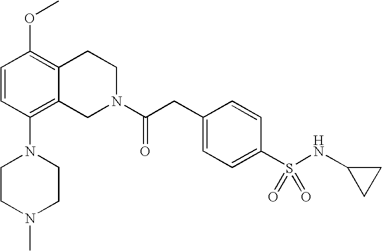

### Example 20a

### (4-Cyclopropylsulfamoyl-phenyl)-acetic acid

Cyclopropylamine (500 □L, 7.21 mmol) was reacted with (4-chlorosulfonyl-phenyl)-acetic acid (200 mg, 0.85 mmol) to give 116 mg of product using the procedure described in example 10b. MS: m/z 256 (M+H).

### Example 20

### N-Cyclopropyl-4-{2-[5-methoxy-8-(4-methyl-piperazin-1-yl)-3,4-dihydro-1H-isoquinolin-2-yl]-2-oxo-ethyl}-benzenesulfonamide

(4-Cyclopropylsulfamoyl-phenyl)-acetic acid (116 mg, 0.46 mmol) was reacted with 5-methoxy-8-(4-methyl-piperizin-1-yl)-1,2,3,4-tetrahydro-isoquinoline (134 mg, 0.51 mmol) using standard HATU coupling conditions (example 8). Product was purified by fcc on silica to give 157 mg of an off-white foam. MS: m/z 499 (M+H).

### Example 21

### N-Cyclobutyl-4-{2-[5-methoxy-8-(4-methyl-piperazin-1-yl)-3,4-dihydro-1H-isoquinolin-2-yl]-2-oxo-ethyl}-benzenesulfonamide

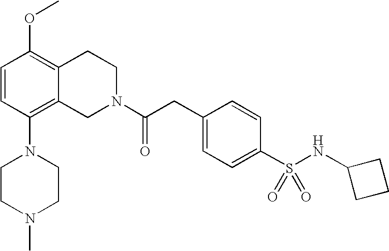

### Example 21a

### (4-Cyclobutylsulfamoyl-phenyl)-acetic acid

Cyclobutylamine (500 □L, 5.87 mmol) was reacted with (4-chlorosulfonyl-phenyl)-acetic acid (200 mg, 0.85 mmol) to give 170 mg of product using the procedure described in example 10b. MS: m/z 270 (M+NH).

### Example 21

### N-Cyclobutyl-4-{2-[5-methoxy-8-(4-methyl-piperazin-1-yl)-3,4-dihydro-1H-isoquinolin-2-yl]-2-oxo-ethyl}-benzenesulfonamide

(4-Cyclobutylsulfamoyl-phenyl)-acetic acid (170 mg, 0.62 mmol) was reacted with 5-methoxy-8-(4-methyl-piperizin-1-yl)-1,2,3,4-tetrahydro-isoquinoline (183 mg, 0.70 mmol) using standard HATU coupling conditions (example 8). Product was purified by fcc on silica to give 239 mg of an off-white foam. MS: m/z 513 (M+H).

### Example 22

### 4-{2-[5-Methoxy-8-(4-methyl-piperazin-1-yl)-3,4-dihydro-1H-isoquinolin-2-yl]-2-oxo-ethyl}-benzenesulfonamide

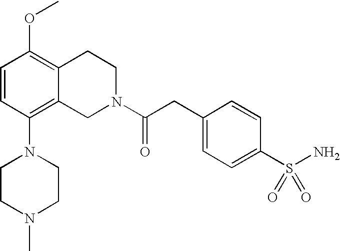

### Example 22

### 4-{2-[5-Methoxy-8-(4-methyl-piperazin-1-yl)-3,4-dihydro-1H-isoquinolin-2-yl]-2-oxo-ethyl}-benzenesulfonamide

(4-Sulfamoyl-phenyl)-acetic acid (134 mg, 0.62 mmol) was reacted with 5-methoxy-8-(4-methyl-piperizin-1-yl)-1,2,3,4-tetrahydro-isoquinoline (162 mg, 0.62 mmol) using standard HATU coupling conditions (example 8). Product was purified by fcc on silica to give 50 mg of an white foam. MS: m/z 459 (M+H).

### Example 22a

### (4-Sulfamoyl-phenyl)-acetic acid

Methanolic ammonia (5 mL saturated solution) was reacted with (4-chlorosulfonyl-phenyl)-acetic acid (241 mg, 1.03 mmol) to give 134 mg of product using a procedure like that described in example 10b. ¹H NMR (DMSO-d₆) δ 12.39 (br s, 1H), 7.76 (d, 2H), 7.44 (d, 2H), 7.28 (s, 2H), 3.67 (s, 2H).

### Example 23

### 4-{2-[5-Methoxy-8-(4-methyl-piperazin-1-yl)-3,4-dihydro-1H-isoquinolin-2-yl]-2-oxo-ethyl}-N-methyl-benzenesulfonamide

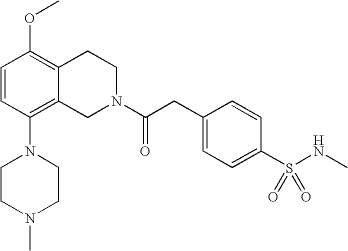

### Example 23a

### (4-Methylsulfamoyl-phenyl)-acetic acid

Methanolic methylamine (10 mL, 2.0 M) was reacted with (4-chlorosulfonyl-phenyl)-acetic acid (239 mg, 1.02 mmol) to give 126 mg of product using the procedure described in example 10b. MS: m/z 230 (M+H).

### Example 23

### 4-{2-[5-Methoxy-8-(4-methyl-piperazin-1-yl)-3,4-dihydro-1H-isoquinolin-2-yl]-2-oxo-ethyl}-N-methyl-benzenesulfonamide

(4-Methylsulfamoyl-phenyl)-acetic acid (126 mg, 0.55 mmol) was reacted with 5-methoxy-8-(-4-methyl-piperizin-1-yl)-1,2,3,4-tetrahydro-isoquinoline (143 mg, 0.55 mmol) using standard HATU coupling conditions (example 8). Product was purified by reverse phase HPLC to give 125 mg of a white foam. MS: m/z 473 (M+H).

### Example 24

### 4-{2-[5-Methoxy-8-(4-methyl-piperazin-1-yl)-3,4-dihydro-1H-isoquinolin-2-yl]-2-oxo-ethyl}-N-ethyl-benzenesulfonamide

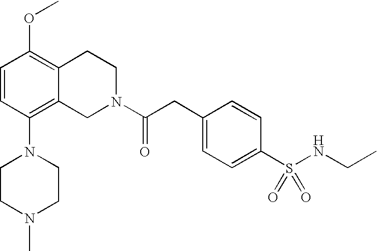

### Example 24a

### (4-Ethylsulfamoyl-phenyl)-acetic acid

Methanolic ethylamine (5 mL, 2.0 M) was reacted with (4-chlorosulfonyl-phenyl)-acetic acid (120 mg, 0.51 mmol) to give 61 mg of product using the procedure described in example 10b. MS: m/z 244 (M+H).

### Example 24

### 4-{2-[5-Methoxy-8-(4-methyl-piperazin-1-yl)-3,4-dihydro-1H-isoquinolin-2-yl]-2-oxo-ethyl}-N-ethyl-benzenesulfonamide

(4-Ethylsulfamoyl-phenyl)-acetic acid (60 mg, 0.25 mmol) was reacted with 5-methoxy-8-(4-methyl-piperizin-1-yl)-1,2,3,4-tetrahydro-isoquinoline (60 mg, 0.23 mmol) using standard HATU coupling conditions (example 8). Product was purified by reverse phase HPLC to give 85 mg of a white foam. MS: m/z 487 (M+H).

### Example 25

### 4-{2-[5-Methoxy-8-(4-methyl-piperazin-1-yl)-3,4-dihydro-1H-isoquinolin-2-yl]-2-oxo-ethyl}-N,N-dimethyl-benzenesulfonamide

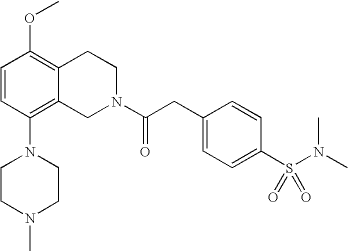

### Example 25a

### (4-Dimethylsulfamoyl-phenyl)-acetic acid

Methanolic dimethylamine (5 mL, 2.0 M) was reacted with (4-chlorosulfonyl-phenyl)-acetic acid (234 mg, 1.00 mmol) to give 211 mg of product using the procedure described in example 10b. MS: m/z 244 (M+H).

### Example 25

### 4-{2-[5-Methoxy-8-(4-methyl-piperazin-1-yl)-3,4-dihydro-1H-isoquinolin-2-yl]-2-oxo-ethyl}-N,N-dimethyl-benzenesulfonamide

(4-Dimethylsulfamoyl-phenyl)-acetic acid (144 mg, 0.59 mmol) was reacted with 5-methoxy-8-(4-methyl-piperizin-1-yl)-1,2,3,4-tetrahydro-isoquinoline (155 mg, 0.59 mmol) using standard HATU coupling conditions (example 8). Product was purified by fcc on silica to give 220 mg of an white foam. MS: m/z 487 (M+H).

### Example 26

### N-Ethyl-4-{2-[5-methoxy-8-(4-methyl-piperazin-1-yl)-3,4-dihydro-1H-isoquinolin-2-yl]-2-oxo-ethyl}-N-methyl-benzenesulfonamide

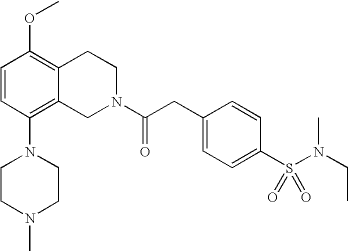

### Example 26a

### [4-(Ethyl-methyl-sulfamoyl)-phenyl]-acetic acid

N-Ethylmethylamine (430 □L, 5.01 mmol) was reacted with (4-chlorosulfonyl-phenyl)-acetic acid (234 mg, 1.00 mmol) to give 237 mg of product using the procedure described in example 10b. MS: m/z 258 (M+H).

### Example 26

### N-Ethyl-4-{2-[5-methoxy-8-(4-methyl-piperazin-1-yl)-3,4-dihydro-1H-isoquinolin-2-yl]-2-oxo-ethyl}-N-methyl-benzenesulfonamide

[4-(Ethyl-methyl-sulfamoyl)-phenyl]-acetic acid (137 mg, 0.53 mmol) was reacted with 5-methoxy-8-(-4-methyl-piperizin-1-yl)-1,2,3,4-tetrahydro-isoquinoline (140 mg, 0.54 mmol) using standard HATU coupling conditions (example 8). Product was purified by fcc on silica to give 214 mg of an off-white foam. MS: m/z 501 (M+H).

### Example 27

### N,N-Diethyl-4-{2-[5-methoxy-8-(4-methyl-piperazin-1-yl)-3,4-dihydro-1H-isoquinolin-2-yl]-2-oxo-ethyl}-benzenesulfonamide

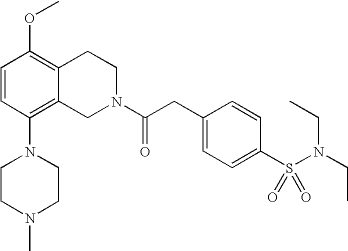

### Example 27a

### [4-(Diethylsulfamoyl)-phenyl]-acetic acid

Diethylamine (520 □L, 5.03 mmol) was reacted with (4-chlorosulfonyl-phenyl)-acetic acid (234 mg, 1.00 mmol) to give 229 mg of product using the procedure described in example 10b. MS: m/z 272 (M+H).

### Example 27

### N,N-Diethyl-4-{2-[5-methoxy-8-(4-methyl-piperazin-1-yl)-3,4-dihydro-1H-isoquinolin-2-yl]-2-oxo-ethyl}-benzenesulfonamide

[4-(Diethylsulfamoyl)-phenyl]-acetic acid (138 mg, 0.51 mmol) was reacted with 5-methoxy-8-(-4-methyl-piperizin-1-yl)-1,2,3,4-tetrahydro-isoquinoline (140 mg, 0.51 mmol) using standard HATU coupling conditions (example 8). Product was purified by fcc on silica to give 200 mg of an off-white foam. MS: m/z 515 (M+H).

### Example 28

### N,N-Dipropyl-4-{2-[5-methoxy-8-(4-methyl-piperazin-1-yl)-3,4-dihydro-1H-isoquinolin-2-yl]-2-oxo-ethyl}-benzenesulfonamide

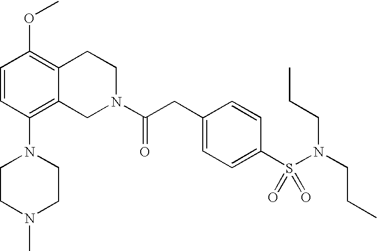

### Example 28a

### [4-(Dipropylsulfamoyl)-phenyl]-acetic acid

Dipropylamine (700 □L, 5.11 mmol) was reacted with (4-chlorosulfonyl-phenyl)-acetic acid (234 mg, 1.00 mmol) to give 234 mg of product using the procedure described in example 10b. MS: m/z 300 (M+H).

### Example 28

### N,N-Dipropyl-4-{2-[5-methoxy-8-(4-methyl-piperazin-1-yl)-3,4-dihydro-1H-isoquinolin-2-yl]-2-oxo-ethyl}-benzenesulfonamide

[4-(Dipropylsulfamoyl)-phenyl)-acetic acid (173 mg, 0.58 mmol) was reacted with 5-methoxy-8-(4-methyl-piperizin-1-yl)-1,2,3,4-tetrahydro-isoquinoline (152 mg, 0.58 mmol) using standard HATU coupling conditions (example 8). Product was purified by fcc on silica to give 240 mg of an off-white foam. MS: m/z 543 (M+H).

### Example 29

### N-Benzyl-4-{2-[5-methoxy-8-(4-methyl-piperazin-1-yl)-3,4-dihydro-1H-isoquinolin-2-yl]-2-oxo-ethyl}-N-methyl-benzenesulfonamide

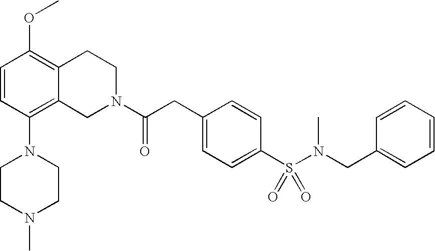

### Example 29a

### (4-(Benzyl-methyl-sulfamoyl)-phenyl]-acetic acid

Benzylmethylamine (650 μL, 5.04 mmol) was reacted with (4-chlorosulfonyl-phenyl)-acetic acid (234 mg, 1.00 mmol) to give 306 mg of product via the procedure described in example 10b. MS: m/z 320+(M+H).

### Example 29

### N-Benzyl-4-{2-[5-methoxy-8-(4-methyl-piperazin-1-yl)-3,4-dihydro-1H-isoquinolin-2-yl]-2-oxo-ethyl}-N-methyl-benzenesulfonamide

[4-(Benzyl-methyl-sulfamoyl)-phenyl]-acetic acid (176 mg, 0.55 mmol) was reacted with 5-methoxy-8-(4-methyl-piperizin-1-yl)-1,2,3,4-tetrahydro-isoquinoline (145 mg, 0.55 mmol) using standard HATU coupling conditions (example 8). Product was purified by fcc on silica to give 207 mg of an off-white foam. MS: m/z 563 (M+H).

### Example 30

### N-Benzyl-4-{2-[5-methoxy-8-(4-methyl-piperazin-1-yl)-3,4-dihydro-1H-isoquinolin-2-yl]-2-oxo-ethyl}-N-ethyl-benzenesulfonamide

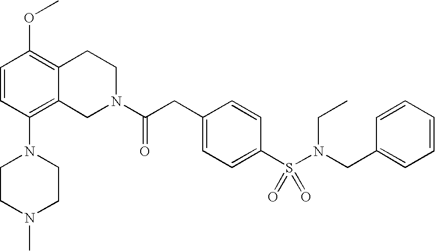

### Example 30a

### [4-(Benzyl-ethyl-sulfamoyl)-phenyl]-acetic acid

Benzylethylamine (750 μL, 5.04 mmol) was reacted with (4-chlorosulfonyl-phenyl)-acetic acid (234 mg, 1.00 mmol) to give 295 mg of product via the procedure described in example 10b. MS: m/z 334 (M+H).

### Example 30

### N-Benzyl-4-{2-[5-methoxy-8-(4-methyl-piperazin-1-yl)-3,4-dihydro-1H-isoquinolin-2-yl]-2-oxo-ethyl}-N-ethyl-benzenesulfonamide

[4-(Benzyl-ethyl-sulfamoyl)-phenyl]-acetic acid (190 mg, 0.57 mmol) was reacted with 5-methoxy-8-(-4-methyl-piperizin-1-yl)-1,2,3,4-tetrahydro-isoquinoline (150 mg, 0.57 mmol) using standard HATU coupling conditions (example 8). Product was purified by fcc on silica to give 240 mg of an off-white foam. MS: m/z 577 (M+H).

### Example 31

### N-Benzyl-4-{2-[5-methoxy-8-(4-methyl-piperazin-1-yl)-3,4-dihydro-1H-isoquinolin-2-yl]-2-oxo-ethyl}-N-isopropyl-benzenesulfonamide

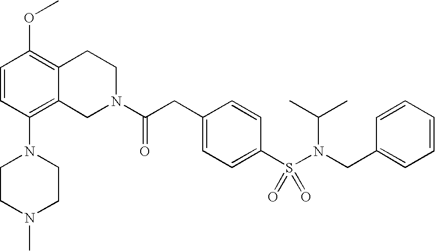

### Example 31a

### [4-(Benzyl-isopropyl-sulfamoyl)-phenyl)-acetic acid

Isopropylbenzylamine (850 □L, 5.08 mmol) was reacted with (4-chlorosulfonyl-phenyl)-acetic acid (234 mg, 1.00 mmol) to give 92 mg of product via the procedure described in example 10b. MS: m/z 348 (M+H).

### Example 31

### N-Benzyl-4-{2-[5-methoxy-8-(4-methyl-piperazin-1-yl)-3,4-dihydro-1H-isoquinolin-2-yl]-2-oxo-ethyl}-N-isopropyl-benzenesulfonamide

[4-(Benzyl-isopropyl-sulfamoyl)-phenyl]-acetic acid (92 mg, 0.26 mmol) was reacted with 5-methoxy-8-(-4-methyl-piperizin-1-yl)-1,2,3,4-tetrahydro-isoquinoline (69 mg, 0.26 mmol) using standard HATU coupling conditions (example 8). Product was purified by fcc on silica to give 83 mg of an off-white foam. MS: m/z 591 (M+H).

### Example 32

### 2-[4-(Azetidine-1-sulfonyl)-phenyl]-1-[5-methoxy-8-(4-methyl-piperazin-1-yl)-3,4-dihydro-1H-isoquinolin-2-yl]-ethanone

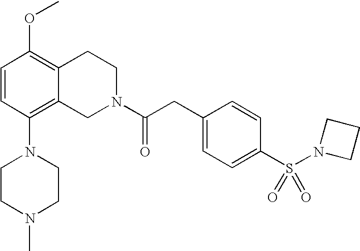

### Example 32a

### [4-(Azetidine-1-sulfonyl)-phenyl]-acetic acid

Azetidine (350 □L, 5.19 mmol) was reacted with (4-chlorosulfonyl-phenyl)-acetic acid (234 mg, 1.00 mmol) to give. 195 mg of product using the procedure described in example 10b. MS: m/z 256 (M+H).

### Example 32

### 2-[4-(Azetidine-1-sulfonyl)-phenyl]-1-[5-methoxy-8-(4-methyl-piperazin-1-yl)-3,4-dihydro-1H-isoquinolin-2-yl]-ethanone

[4-(Azetidine-1-sulfonyl)-phenyl]-acetic acid (119 mg, 0.47 mmol) was reacted with 5-methoxy-8-(4-methyl-piperizin-1-yl)-1,2,3,4-tetrahydro-isoquinoline (122 mg, 0.47 mmol) using standard HATU coupling conditions (example 8). Product was purified by fcc on silica to give 162 mg of an off-white foam. MS: m/z 499 (M+H).

### Example 33

### 1-[5-Methoxy-8-(4-methyl-piperazin-1-yl)-3,4-dihydro-1H-isoquinolin-2-yl]-2-[4-(pyrrolidine-1 sulfonyl)-phenyl]-ethanone

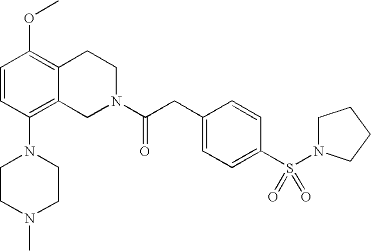

### Example 33a

### [4-(Pyrrolidine-1-sulfonyl)-phenyl]-acetic acid

Pyrrolidine (420 □L, 5.02 mmol) was reacted with (4-chlorosulfonyl-phenyl)-acetic acid (234 mg, 1.00 mmol) to give 186 mg of product using a procedure like that described in example 10b. MS: m/z 270 (M+H).

### Example 33

### 1-[5-Methoxy-8-(4-methyl-piperazin-1-yl)-3,4-dihydro-1H-isoquinolin-2-yl]-2-[4-(pyrrolidine-1-sulfonyl)-phenyl]-ethanone

[4-(Pyrrolidine-1-sulfonyl)-phenyl]-acetic acid (186 mg, 0.69 mmol) was reacted with 5-methoxy-8-(4-methyl-piperizin-1-yl)-1,2,3,4-tetrahydro-isoquinoline (181 mg, 0.69 mmol) using standard HATU coupling conditions (example 8). Product was purified by fcc on silica to give 115 mg of an off-white foam. MS: m/z 513 (M+H).

### Example 34

### N-{2-[5-Methoxy-8-(4-methyl-piperazin-1-yl)-3,4-dihydro-1H-isoquinolin-2-yl]-2-oxo-ethyl}-isonicotinamide

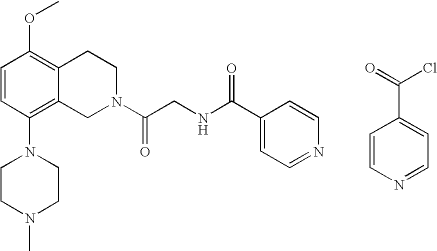

### Example 34a

### Isonicotinoyl chloride hydrochloride

To a slurry of isonicotinic acid (170 mg, 1.38 mmol) in 10 mL DCM containing 2 drops of DMF was added oxalyl chloride (180 μL, 2.10 mmol). Mixture was stirred for 2 h and all solvent was evaporated under reduced pressure. Product was then pumped down under high vacuum for 1.5 h, dissolved in DCM, and used in the next step without purification.

### Example 34b

### {2-[5-Methoxy-8-(4-methyl-piperazin-1-yl)-3,4-dihydro-1H-isoquinolin-2-yl]-2-oxo-ethyl}-carbamic acid tert-butyl ester

5-Methoxy-8-(4-methyl-piperizin-1-yl)-1,2,3,4-tetrahydro-isoquinoline (5.28 g, 20.2 mmol) and N-(tert-butoxycarbonyl)-glycine (3.50 g, 20.0 mmol) were combined in 150 mL DCM containing (4.5 mL, 32 mmol) Et₃N. To this was added 7.60 g (20.0 mmol) HATU. Mixture was stirred for 18 h, diluted with DCM (200 mL), and extracted with 1N HCl (150 mL) then with 20% K₂CO_{3(aq)} (2×150 mL). Organic layer was dried over Na₂SO₄, filtered, and solvent was evaporated under reduced pressure. Product was triturated in IPE (50 mL) for 18 h, filtered, and washed with cold IPE to give 6.99 g of a white powder. MS: m/z 419 (M+H).

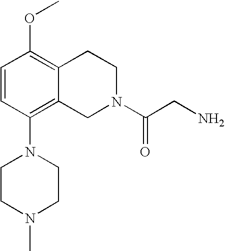

### Example 34c

### 2-Amino-1-[5-methoxy-8-(4-methyl-piperazin-1-yl)-3,4-dihydro-1H-isoquinolin-2-yl]-ethanone

{2-[5-Methoxy-8-(4-methyl-piperazin-1-yl)-3,4-dihydro-1H-isoquinolin-2-yl]-2-oxo-ethyl}-carbamic acid tert-butyl ester (6.99 g, 16.7 mmol) was dissolved in 100 mL DCM and to this was added 20 mL TFA. Mixture was stirred for 6 h and solvent/A was evaporated under reduced pressure. Residue was mixed with 50 mL 25% KOH_{(aq)} (pH 14) and extracted with CHCl₃ (4×75 mL). Extracts were combined, dried over Na₂SO₄, filtered, and evaporated under reduced pressure. This gave 5.40 g (quantitative yield) of crude material which was used without purification. MS: m/z 319 (M+H).

### Example 34

### N-{2-[5-Methoxy-8-(4-methyl-piperazin-1-yl)-3,4-dihydro-1H-isoquinolin-2-yl]-2-oxo-ethyl}-isonicotinamide

To a stirred solution of 2-amino-1-[5-methoxy-8-(4-methyl-piperazin-1-yl)-3,4-dihydro-1H-isoquinolin-2-yl]-ethanone (108 mg, 0.34 mmol) in 10 mL DCM containing 150 μL (1.08 mmol) Et₃N was added isonicotinoyl chloride hydrochloride (61 mg, 0.34 mmol). Mixture was stirred for 18 h, diluted with DCM (20 mL), and extracted with 20% K₂CO_{3(aq)} (2×10 mL). Organic layer was dried over Na₂SO₄, filtered, and solvent was evaporated under reduced pressure. Product was purified by preparative reverse phase chromatography to give 68 mg of a foam. MS: m/z 424 (M+H).

### Example 35

### N-{4-[5-Methoxy-8-(4-methyl-piperazin-1-yl)-3,4-dihydro-1H-isoquinolin-2-yl]-4-oxo-butyl}-isonicotinamide

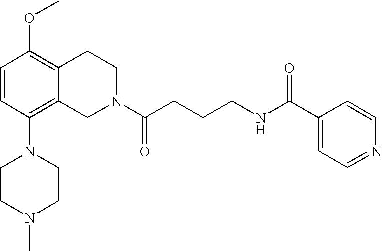

### Example 35a

### {4-[5-Methoxy-8-(4-methyl-piperazin-1-yl)-3,4-dihydro-1H-isoquinolin-2-yl)-4-oxo-butyl}-carbamic acid tert-butyl ester

5-Methoxy-8-(-4-methyl-piperizin-1-yl)-1,2,3,4-tetrahydro-isoquinoline (5.28 g, 20.2 mmol) was coupled with 4-(tert-butoxycarbonylamino)-butyric acid (4.06 g, 20.0 mmol) using HATU following a procedure outlined in example 34b. Product was purified by fcc on silica to give 8.69 g of an oil. MS: m/z 447 (M+H).

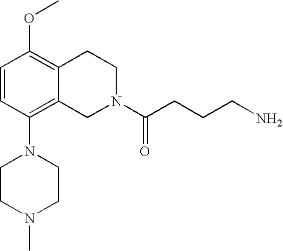

### Example 35b

### 4-Amino-1-[5-methoxy-8-(4-methyl-piperazin-1-yl)-3,4-dihydro-1H-isoquinolin-2-yl]-butan-1-one

{4-[5-Methoxy-8-(4-methyl-piperazin-1-yl)-3,4-dihydro-1H-isoquinolin-2-yl]-4-oxo-butyl}-carbamic acid tert-butyl ester (8.69 g, 19.5 mmol) was deprotected using a procedure similar to that described in example 34c. This gave 6.59 g of crude material which was used without purification. MS: m/z 347 (M+H).

### Example 35

### N-(4-[5-Methoxy-8-(4-methyl-piperazin-1-yl)-32,4-dihydro-1H-isoquinolin-2-yl]-4-oxo-butyl}-isonicotinamide

4-Amino-1-[5-methoxy-8-(4-methyl-piperazin-1-yl)-3,4-dihydro-1H-isoquinolin-2-yl]-butan-1-one (117 mg, 0.34 mmol) was reacted with isonicotinoyl chloride hydrochloride (61 mg, 0.34 mmol) using a procedure similar to that described in example 34. Product was purified by preparative reverse phase chromatography to give 88 mg of a foam. MS: m/z 452 (M+H).

### Example 36

### N-{5-[5-Methoxy-8-(4-methyl-piperazin-1-yl)-3,4-dihydro-1H-isoquinolin-2-yl]-5-oxo-pentyl}-isonicotinamide

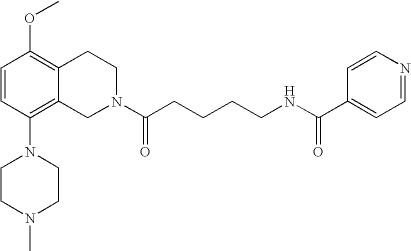

### Example 36a

### {5-[5-Methoxy-8-(4-methyl-piperazin-1-yl)-3,4-dihydro-1H-isoquinolin-2-yl]-5-oxo-pentyl}-carbamic acid tert-butyl ester

5-Methoxy-8-(4-methyl-piperizin-1-yl)-1,2,3,4-tetrahydro-isoquinoline (5.28 g, 20.2 mmol) was coupled with 5-(tert-Butoxycarbonylamino)-valeric acid (4.34 g, 20.0 mmol) using HATU following a procedure outlined in example 34b. Product was purified by fcc on silica to give 7.41 g of an oil. MS: m/z 461 (M+H).

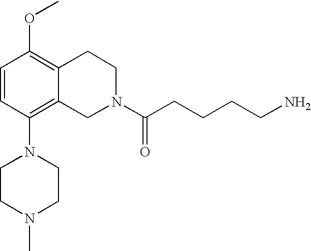

### Example 36b

### 5-Amino-1-[5-methoxy-8-(4-methyl-piperazin-1-yl)-3,4-dihydro-1H-isoquinolin-2-yl]-pentan-1-one

{5-[5-Methoxy-8-(4-methyl-piperazin-1-yl)-3,4-dihydro-1H-isoquinolin-2-yl]-5-oxo-pentyl}-carbamic acid tert-butyl ester (7.41 g, 16.1 mmol) was deprotected using a procedure similar to that described in example 34c. This gave 5.85 g of crude material which was used without purification. MS: m/z 361 (M+H).

### Example 36

### N-{5-[5-Methoxy-8-(4-methyl-piperazin-1-yl)-3,4-dihydro-1H-isoquinolin-2-yl]-5-oxo-pentyl}-isonicotinamide

5-Amino-1-[5-methoxy-8-(4-methyl-piperazin-1-yl)-3,4-dihydro-1H-isoquinolin-2-yl]-pentan-1-one (122 mg, 0.34 mmol) was reacted with isonicotinoyl chloride hydrochloride (61 mg, 0.34 mmol) using a procedure similar to that described in example 34. Product was purified by preparative reverse phase chromatography to give 113 mg of a gum. MS: m/z 466 (M+H).

### Example 37

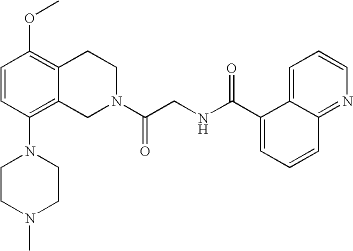

### Example 37a

### Trifluoro-methanesulfonic acid quinolin-5-yl ester

To a solution of 5-hydroxyquinoline (2.05 g, 14.1 mmol) in DCM (40 mL) was added triethylamine (4.0 mL, 29 mmol) followed by trifluoromethanesulfonic anhydride (2.4 mL, 14 mmol) at 0° C. The reaction mixture was stirred at room temperature for 48 h, diluted with DCM (40 mL), extracted with saturated NaHCO₃ (3×30 mL). The combined organic extracts were dried over NaSO₄, filtered and concentrated. The crude material was purified by fcc (10:1, DCM:EtOAc) to give 1.26 g of product. MS m/z 278 (M+H).

### Example 37b

### Quinoline-5-carboxylic acid methyl ester

To a mixture of DMSO (20 mL) and MeOH (20 mL) was added BINAP (187 mg, 0.46 mmol), Pd(OAc)₂ (101 mg, 0.45 mmol), trifluoro-methanesulfonic acid quinolin-5-yl ester (1.26 g, 4.46 mmol), and 640 μL (4.59 mmol) Et₃N. Mixture was purged with CO (via 18 ga. needle and balloon) for 40 minutes and heated to 70° C. Mixture was kept under CO atmosphere (atmospheric pressure) for 20 h at 70° C. At this time mixture was poured into 100 mL of 1:1 EtOAc:Et₂O, extracted with H₂O (3×50 mL), dried over NaSO₄, filtered, and concentrated. Residue was purified by chromatography (10:1, DCM:EtOAc) to give 0.52 g of product. MS m/z 188 (M+H).

### Example 37c

### Quinoline-5-carboxylic acid hydrochloride

Quinoline-5-carboxylic acid methyl ester (1.02 g, 5.43 mmol) was suspended in 10 mL 6N HCl and heated to 110° C. for 18 h. The hot solution was cooled 0° C. for 1 h, filtered, and product was dried under high vacuum for 18 h to give 0.90 g of a tan powder. MS m/z 174 (M+H).

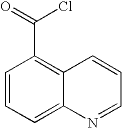

### Example 37d

### Quinoline-5-carbonyl chloride hydrochloride

Quinoline-5-carboxylic acid hydrochloride (280 mg, 1.34 mmol) was reacted with oxalyl chloride (180 μL, 2.10 mmol) using a procedure similar to that described in example 34a. Product was then pumped down under high vacuum for 1.5 h, dissolved in DCM, and used in the next step without purification

### Example 37

### Quinoline-5-carboxylic acid {2-[5-methoxy-8-(4-methyl-piperazin-1-yl)-3,4-dihydro-1H-isoquinolin-2-yl]-2-oxo-ethyl}-amide

2-Amino-1-[5-methoxy-8-(4-methyl-piperazin-1-yl)-3,4-dihydro-1H-isoquinolin-2-yl]-ethanone (107 mg, 0.34 mmol) was reacted with quinoline-5-carbonyl chloride hydrochloride (78 mg, 0.34 mmol) using a procedure similar to that described in example 34. Product was purified by preparative reverse phase chromatography to give 83 mg of a foam. MS: m/z 474 (M+H).

### Example 38

### Quinoline-5-carboxylic acid {4-[5-methoxy-8-(4-methyl-piperazin-1-yl)-3,4-dihydro-1H-isoquinolin-2-yl]-4-oxo-butyl}-amide

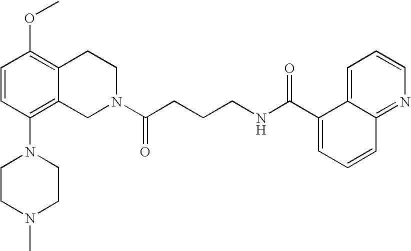

### Example 38

### Quinoline-5-carboxylic acid {4-[5-methoxy-8-(4-methyl-piperazin-1-yl)-3,4-dihydro-1H-isoquinolin-2-yl]-4-oxo-butyl}-amide

4-Amino-1-[5-methoxy-8-(4-methyl-piperazin-1-yl)-3,4-dihydro-1H-isoquinolin-2-yl]-butan-1-one (117 mg, 0.34 mmol) was reacted with quinoline-5-carbonyl chloride hydrochloride (78 mg, 0.34 mmol) using a procedure similar to that described in example 34. Product was purified by preparative reverse phase chromatography to give 89 mg of a foam. MS: m/z 502 (M+H).

### Example 39

### Quinoline-5-carboxylic acid {5-[5-methoxy-8-(4-methyl-piperazin-1-yl)-3,4-dihydro-1H-isoquinolin-2-yl]-5-oxo-pentyl}-amide

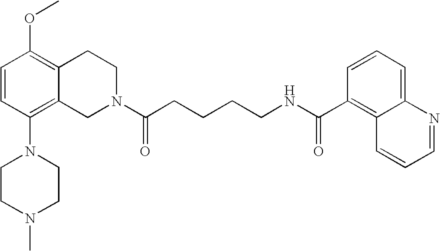

### Example 39

### Quinoline-5-carboxylic acid {5-[5-methoxy-8-(4-methyl-piperazin-1-yl)-3,4-dihydro-1H-isoquinolin-2-yl]-5-oxo-pentyl}-amide

5-Amino-1-[5-methoxy-8-(4-methyl-piperazin-1-yl)-3,4-dihydro-1H-isoquinolin-2-yl]-pentan-1-one (122 mg, 0.34 mmol) was reacted with quinoline-5-carbonyl chloride hydrochloride (78 mg, 0.34 mmol) using a procedure similar to that described in example 34. Product was purified by preparative reverse phase chromatography to give 122 mg of a foam. MS: m/z 516 (M+H).

### Example 40

### N-{2-[5-Methoxy-8-(4-methyl-piperazin-1-yl)-3,4-dihydro-1H-isoquinolin-2-yl]-2-oxo-ethyl}-benzamide

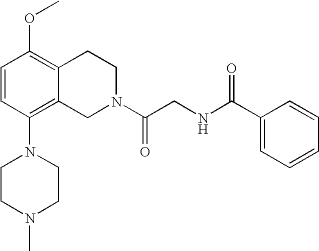

### Example 40

### N-{2-[5-Methoxy-8-(4-methyl-piperazin-1-yl)-3,4-dihydro-1H-isoquinolin-2-yl]-2-oxo-ethyl}-benzamide

2-Amino-1-[5-methoxy-8-(4-methyl-piperazin-1-yl)-3,4-dihydro-1H-isoquinolin-2-yl]-ethanone (107 mg, 0.34 mmol) was reacted with benzoyl chloride (45 μL, 0.39 mmol) using a procedure similar to that described in example 34. Product was purified by preparative reverse phase chromatography to give 77 mg of a foam. MS: m/z 423 (M+H).

### Example 41

### N-{3-[5-Methoxy-8-(4-methyl-piperazin-1-yl)-3,4-dihydro-1H-isoquinolin-2-yl]-3-oxo-propyl}-benzamide

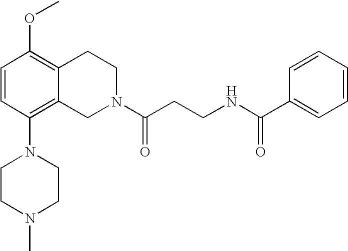

### Example 41a

### {3-[5-Methoxy-8-(4-methyl-piperazin-1-yl)-3,4-dihydro-1H-isoquinolin-2-yl]-3-oxo-propyl}-carbamic acid tert-butyl ester

5-Methoxy-8-(4-methyl-piperizin-1-yl)-1,2,3,4-tetrahydro-isoquinoline (5.28 g, 20.2 mmol) was coupled with N-(tert-butoxycarbonyl)-β-alanine (3.78 g, 20.0 mmol) using HATU following a procedure outlined in example 34b. Product was triturated in IPE (50 mL) for 18 h, filtered, and washed with cold IPE to give 5.23 g of a white powder. MS: m/z 433 (M+H).

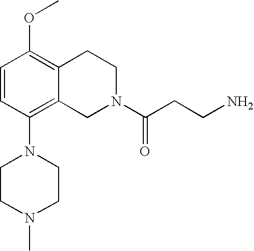

### Example 41b

### 3-Amino-1-[5-methoxy-8-(4-methyl-piperazin-1-yl)-3,4-dihydro-1H-isoquinolin-2-yl]-propan-1-one

{3-[5-Methoxy-8-(4-methyl-piperazin-1-yl)-3,4-dihydro-1H-isoquinolin-2-yl]-3-oxo-propyl}-carbamic acid tert-butyl ester (5.23 g, 12.1 mmol) was deprotected using a procedure similar to that described in example 34c. This gave 4.04 g (quantitative yield) of crude material which was used without purification. MS: m/z 333 (M+H).

### Example 41

### N-{3-(5-Methoxy-8-(4-methyl-piperazin-1-yl)-3,4-dihydro-1H-isoquinolin-2-yl]-3-oxo-propyl}-benzamide

3-Amino-1-[5-methoxy-8-(4-methyl-piperazin-1-yl)-3,4-dihydro-1H-isoquinolin-2-yl]-propan-1-one (112 mg, 0.34 mmol) was reacted with benzoyl chloride (45 μL, 0.39 mmol) using a procedure similar to that described in example 34. Product was purified by preparative reverse phase chromatography to give 65 mg of a foam. MS: m/z 437 (M+H).

### Example 42

### N-{4-(5-Methoxy-8-(4-methyl-piperazin-1-yl)-3,4-dihydro-1H-isoquinolin-2-yl]-4-oxo-butyl}-benzamide

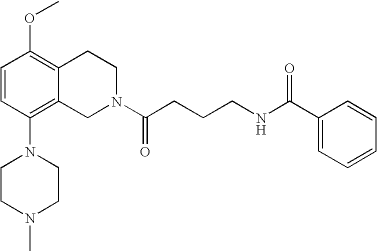

### Example 42

### N-{4-[5-Methoxy-8-(4-methyl-piperazin-1-yl)-3,4-dihydro-1H-isoquinolin-2-yl]-4-oxo-butyl}-benzamide

4-Amino-1-[5-methoxy-8-(4-methyl-piperazin-1-yl)-3,4-dihydro-1H-isoquinolin-2-yl]-butan-1-one (117 mg, 0.34 mmol) was reacted with benzoyl chloride (45 μL, 0.39 mmol) using a procedure similar to that described in example 34. Product was purified by preparative reverse phase chromatography to give 77 mg of a gum. MS: m/z 451 (M+H).

### Example 43

### N-{5-[5-Methoxy-8-(4-methyl-piperazin-1-yl)-3,4-dihydro-1H-isoquinolin-2-yl]-5-oxo-pentyl}-benzamide

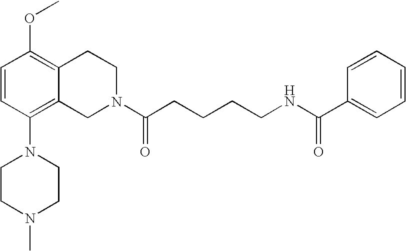

### Example 43

### N-{5-[5-Methoxy-8-(4-methyl-piperazin-1-yl)-3,4-dihydro-1H-isoquinolin-2-yl]-5-oxo-pentyl}-benzamide

5-Amino-1-[5-methoxy-8-(4-methyl-piperazin-1-yl)-3,4-dihydro-1H-isoquinolin-2-yl]-pentan-1-one (122 mg, 0.34 mmol) was reacted with benzoyl chloride (45 μL, 0.39 mmol) using a procedure similar to that described in example 34. Product was purified by preparative reverse phase chromatography to give 24 mg of a foam. MS: m/z 465 (M+H).

### Example 44

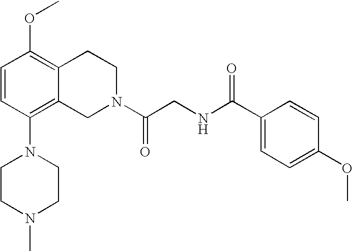

### Example 44

### 4-Methoxy-N-{2-[5-methoxy-8-(4-methyl-piperazin-1-yl)-3,4-dihydro-1H-isoquinolin-2-yl]-2-oxo-ethyl}-benzamide

2-Amino-1-[5-methoxy-8-(4-methyl-piperazin-1-yl)-3,4-dihydro-1H-isoquinolin-2-yl]-ethanone (107 mg, 0.34 mmol) was reacted with p-anisoyl chloride (50 μL, 0.37 mmol) using a procedure similar to that described in example 34. Product was purified by preparative reverse phase chromatography to give 87 mg of a foam. MS: m/z 453 (M+H).

### Example 45

### 4-Methoxy-N-{4-[5-methoxy-8-(4-methyl-piperazin-1-yl)-3,4-dihydro-1H-isoquinolin-2-yl]-4-oxo-butyl}-benzamide

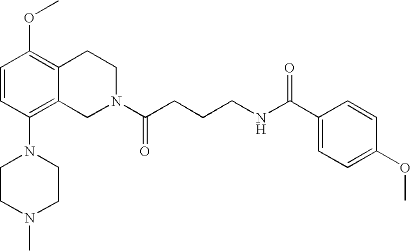

### Example 45

### 4-Methoxy-N-{4-[5-methoxy-8-(4-methyl-piperazin-1-yl)-3,4-dihydro-1H-isoquinolin-2-yl]-4-oxo-butyl}-benzamide

4-Amino-1-[5-methoxy-8-(4-methyl-piperazin-1-yl)-3,4-dihydro-1H-isoquinolin-2-yl]-butan-1-one (117 mg, 0.34 mmol) was reacted with p-anisoyl chloride (50 μL, 0.37 mmol) using a procedure similar to that described in example 34. Product was purified by preparative reverse phase chromatography to give 71 mg of a gum. MS: m/z 481 (M+H).

### Example 46

### 4-Methoxy-N-{5-[5-methoxy-8-(4-methyl-piperazin-1-yl)-3,4-dihydro-1H-isoquinolin-2-yl]-5-oxo-pentyl}-benzamide

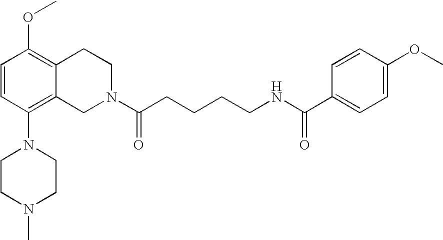

### Example 46

### 4-Methoxy-N-{5-[5-methoxy-8-(4-methyl-piperazin-1-yl)-3,4-dihydro-1H-isoquinolin-2-yl]-5′-oxo-pentyl}-benzamide

5-Amino-1-[5-methoxy-8-(4-methyl-piperazin-1-yl)-3,4-dihydro-1H-isoquinolin-2-yl]-pentan-1-one (122 mg, 0.34 mmol) was reacted with p-anisoyl chloride (50 μL-0.37 mmol) using a procedure similar to that described in example 34. Product was purified by preparative reverse phase chromatography to give 81 mg of a foam. MS: m/z 495 (M+H).

### Example 47

### (4-Butylamino-phenyl)-[5-methoxy-8-(4-methyl-piperazin-1-yl)-3,4-dihydro-1H-isoquinolin-2-yl]-methanone

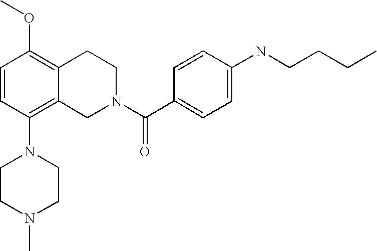

### Example 47

### (4-Butylamino-phenyl)-[5-methoxy-8-(4-methyl-piperazin-1-yl)-3,4-dihydro-1H-isoquinolin-2-yl]-methanone

4-(Butylamino)benzoic acid (116 mg, 0.600 mmol, Aldrich) was treated with a solution of previously prepared (see 11427-50-2) 5-methoxy-8-(4-methyl-piperazin-1-yl)-1,2,3,4-tetrahydro-isoquinoline (131 mg, 0.500 mmol) in anhydrous dichloromethane (2.5 mL). Triethylamine (0.174 mL, 0.126 g, 1.25 mmol) was introduced via pipet followed by the addition of HATU (247 mg, 0.650 mmol), and the entire mixture was diluted with dichloromethane to a total volume of 12 mL. The reaction mixture was agitated at room temperature 18 h; then diluted to 30 mL with dichloromethane. An equal volume of 20% aqueous potassium carbonate was added. The organic phase was removed, and the aqueous portion was extracted with dichloromethane (30 mL). The combined organics were dried over sodium sulfate, filtered, and concentrated leaving the crude product, which was purified by fcc on a 5 g silica gel column. The desired fractions were collected, concentrated under vacuum, and dried under high vacuum overnight leaving 217.6 mg (>99%) of orange foam.

LC/MS (M+1) m/z=437.

### Example 48

### (4-Cyclohexyl-phenyl)-[5-methoxy-8-(4-methyl-piperazin-1-yl)-3,4-dihydro-1H-isoquinolin-2-yl]-methanone

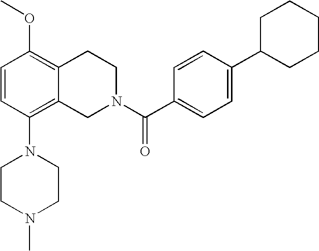

### Example 48

### (4-Cyclohexyl-phenyl)-[5-methoxy-8-(4-methyl-piperazin-1-yl)-3,4-dihydro-1H-isoquinolin-2-yl]-methanone

4-Cyclohexylbenzoic acid (123 mg, 0.602 mmol, Lancaster) was treated with a solution of previously prepared (see 11427-50-2) 5-methoxy-8-(4-methyl-piperazin-1-yl)-1,2,3,4-tetrahydro-isoquinoline (131 mg, 0.503 mmol) in anhydrous dichloromethane (2.5 mL). Triethylamine (0.117 mL, 85.2 mg, 0.842 mmol) was introduced via pipet followed by the addition of HATU (247 mg, 0.650 mmol), and the entire mixture was diluted with dichloromethane (7.5 mL). The reaction mixture was agitated at room temperature 18 h; then diluted to 30 mL with dichloromethane. An equal volume of 20% aqueous potassium carbonate was added. The organic phase was removed, and the aqueous portion was extracted with dichloromethane (20 mL). The combined organics were dried over sodium sulfate, filtered, and concentrated leaving the crude product, which was purified fcc on 5 g silica gel. The desired fractions were collected, concentrated under vacuum, and dried under high vacuum overnight leaving 174 mg (78%) of yellow foam. LC/MS (M+1) m/z=448.

### Example 49

### (4-Benzyl-phenyl)-[5-methoxy-8-(4-methyl-piperazin-1-yl)-3,4-dihydro-1H-isoquinolin-2-yl]-methanone

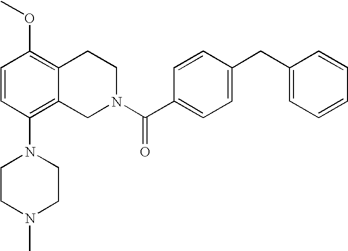

### Example 49

### (4-Benzyl-phenyl)-[5-methoxy-8-(4-methyl-piperazin-1-yl)-3,4-dihydro-1H-isoquinolin-2-yl]-methanone

This compound was synthesized from 5-methoxy-8-(4-methyl-piperazin-1-yl)-1,2,3,4-tetrahydro-isoquinoline (11427-50-2) and diphenylmethane-4-carboxylic acid (Trans World Chemicals), using the same synthetic procedures, scale, and stoichiometry as demonstrated in Example 2 above. Yield: 170 mg (75%), yellow foam. LC/MS (M+1) m/z=456.

### Example 50

### (4′-Ethyl-biphenyl-4-yl)-[5-methoxy-8-(4-methyl-piperazin-1-yl)-3,4-dihydro-1H-isoquinolin-2-yl]-methanone

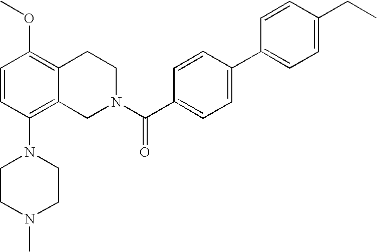

### Example 50

### (4′-Ethyl-biphenyl-4-yl)-[5-methoxy-8-(4-methyl-piperazin-1-yl)-3,4-dihydro-1H-isoquinolin-2-yl]-methanone

This compound was synthesized from 5-methoxy-8-(4-methyl-piperazin-1-yl)-1,2,3,4-tetrahydro-isoquinoline (11427-50-2) and 4-ethylbiphenyl-4′-carboxylic acid (Acros), using the same synthetic procedures, scale, and stoichiometry as demonstrated in Example 2 above. Yield: 177 mg (76%), yellow foam. LC/MS (M+1) m/z=470.

### Example 51

### (4′-Hydroxy-biphenyl-4-yl)-[5-methoxy-8-(4-methyl-piperazin-1-yl)-3,4-dihydro-1H-isoquinolin-2-yl]-methanone

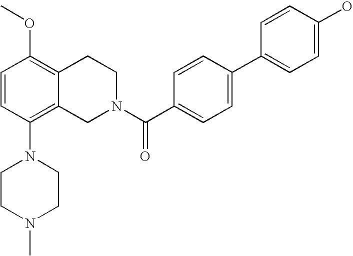

### Example 51

### (4′-Hydroxy-biphenyl-4-yl)-[5-methoxy-8-(4-methyl-piperazin-1-yl)-3,4-dihydro-1H-isoquinolin-2-yl]-methanone

This compound was synthesized from 5-methoxy-8-(4-methyl-piperazin-1-yl)-1,2,3,4-tetrahydro-isoquinoline (11427-50-2) and 4′-hydroxy-4-biphenylcarboxylic acid (Aldrich), using the same synthetic procedures, scale, and stoichiometry as demonstrated in Example 2 above. Yield: 115 mg (50%), pale orange foam.

LC/MS (M+1) m/z=458.

### Example 52

### [5-Methoxy-8-(4-methyl-piperazin-1-yl)-3,4-dihydro-1H-isoquinolin-2-yl]-(4-phenoxy-phenyl)-methanone

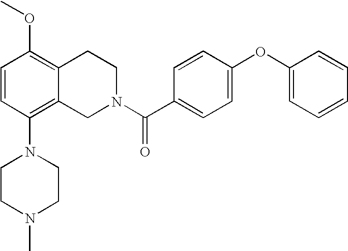

### Example 52

### [5-Methoxy-8-(4-methyl-piperazin-1-yl)-3,4-dihydro-1H-isoquinolin-2-yl]-(4-phenoxy-phenyl)-methanone

4-Phenoxybenzoic acid (129 mg, 0.602 mmol, Trans World Chemicals) was treated with a solution of previously prepared (11427-50-2) 5-methoxy-8-(4-methyl-piperazin-1-yl)-1,2,3,4-tetrahydro-isoquinoline (131 mg, 0.503 mmol) in anhydrous dichloromethane (2.5 mL). Triethylamine (0.117 mL, 85.2 mg, 0.842 mmol) was introduced via pipet followed by the addition of HATU (247 mg, 0.650 mmol), and the entire mixture was diluted with dichloromethane (7.5 mL). The reaction mixture was agitated at room temperature 18 h, then diluted to 30 mL with dichloromethane. An equal volume of 20% aqueous potassium carbonate was added. The organic phase was removed, and the aqueous portion was extracted with dichloromethane (20 mL). The combined organics were dried over sodium sulfate, filtered, and concentrated leaving the crude product, which was purified by fcc. The desired fractions were collected, concentrated under vacuum, and dried under high vacuum overnight leaving 189 mg (83%) of orange gum. LC/MS (M+1) m/z=458.

### Example 53

### (4-Benzoyl-phenyl)-[5-methoxy-8-(4-methyl-piperazin-1-yl)-3,4-dihydro-1H-isoquinolin-2-yl]-methanone

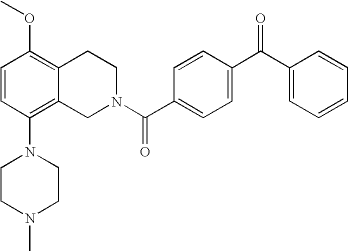

### Example 53

### (4-Benzoyl-phenyl)-[5-methoxy-8-(4-methyl-piperazin-1-yl)-3,4-dihydro-1H-isoquinolin-2-yl]-methanone

4-Benzoylbenzoic acid (136 mg, 0.601 mmol, Aldrich) was treated with a solution of previously prepared (11427-50-2) 5-methoxy-8-(4-methyl-piperazin-1-yl)-1,2,3,4-tetrahydro-isoquinoline (131 mg, 0.503 mmol) in anhydrous dichloromethane (2.5 mL). Triethylamine (0.117 mL, 85.2 mg, 0.842 mmol) was introduced via pipet followed by the addition of HATU (247 mg, 0.650 mmol), and the entire mixture was diluted with dichloromethane (7.5 mL). The reaction mixture was agitated at room temperature 18 h, then diluted to 30 mL with dichloromethane. An equal volume of 20% aqueous potassium carbonate was added. The organic phase was removed, and the aqueous portion was extracted with dichloromethane (20 mL). The combined organics were dried over sodium sulfate, filtered, and concentrated leaving the crude product, which was purified by fcc. The desired fractions were collected, concentrated under vacuum, and dried under high vacuum overnight leaving 211 mg (90%) of pale yellow foam. LC/MS (M+1) m/z=470.

### Example 54

### 5-Methoxy-8-(4-methyl-piperazin-1-yl)-3,4-dihydro-1H-isoquinoline-2-carboxylic acid [4-(4-methoxy-phenylsulfamoyl)-phenyl]-amide

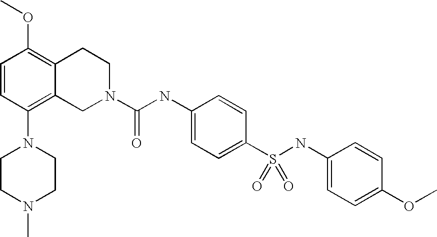

### Example 54a

### N-(4-Methoxy-phenyl)-4-nitro-benzenesulfonamide

N-4-Methoxy-phenyl)-4-nitro-benzenesulfonamide (6.15 g, 19.9 mmol, as prepared in Example 9) was suspended in ethyl acetate (50 mL) and ethanol (50 mL). This suspension was treated with stannous chloride dihydrate (24.2 g, 107 mmol), and the mixture was subsequently heated to reflux for 35 min at which time the reaction was complete. The mixture was cooled to room temperature then poured into ice and treated with 10% aqueous sodium hydroxide until basic. After standing 2 h, the mixture was filtered through diatomaceous earth (washing with aqueous saturated sodium bicarbonate and ethyl acetate). The biphasic mixture was separated, the aqueous portion was extracted with ethyl acetate (1×100 mL), and the combined organics were washed with brine, dried (sodium sulfate), filtered and concentrated leaving 4.04 g (73%) pale purple solid, which required no further purification. LC/MS (M+1) m/z=279.

### Example 54b

### N-(4-Methoxy-phenyl)-4-nitro-benzenesulfonamide

4-Nitrobenzenesulfonyl chloride (5.01 g, 22.6 mmol, Acros) was treated with p-anisidine (25.15 g, 204.2 mmol, Aldrich) in methanol (100 mL). After 1 h, the reaction was complete, and the mixture was concentrated under reduced pressure leaving a purplish-brown solid. The solid was recrystallized from ethanol leaving 5.59 g (80%) silver-black, flaky solid.

¹H NMR (CDCl₃) δ 8.27 (d, 2H), 7.86 (d, 2H), 6.97 (d, 2H), 6.79 (d, 2H), 6.45 (s, 1H), 3.77 (s, 3H).

### Example 54

### 5-Methoxy-8-(4-methyl-piperazin-1-yl)-3,4-dihydro-1H-isoquinoline-2-carboxylic acid [4-(4-methoxy-phenylsulfamoyl)-phenyl]-amide

4-Amino-4′-methoxybenzenesulfonanilide (0.139 g, 0.500 mmol, see example 10, 11837-31-1,) was suspended in dichloromethane (3.0 mL); then DMF (about 1-2 mL) was added until all solids dissolved. The solution was treated with 1,1′carbonyldiimidazole (0.123 g, 0.760 mmol, Aldrich) and stirred at room temperature 16 h. The previously prepared (11427-50-2) 5-methoxy-8-(4-methyl-piperazin-1-yl)-1,2,3,4-tetrahydro-isoquinoline (0.134 g, 0.513 mmol) was added, and the solution was stirred 1 h. The reaction mixture was diluted with ethyl acetate (20 mL) and washed with 20% aqueous potassium carbonate (3×25 mL). The organic portion was dried over sodium sulfate, filtered and concentrated leaving a yellow-white semi-solid. The crude product was purified by fcc leaving 211 mg (75%) white solid.

LC/MS (M+1) m/z=566.

### Example 55

### 5-Methoxy-8-(4-methyl-piperazin-1-yl)-3,4-dihydro-1H-isoquinoline-2-carboxylic acid (4-phenylsulfamoyl-phenyl)-amide

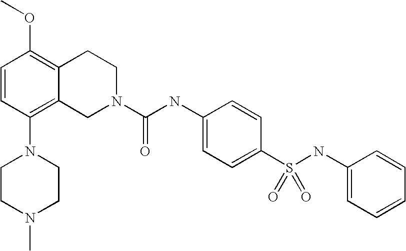

### Example 55

### 5-Methoxy-8-(4-methyl-piperazin-1-yl)-3,4-dihydro-1H-isoquinoline-2-carboxylic acid (4-phenylsulfamoyl-phenyl)-amide

4-(Chlorosulfonyl)phenyl isocyanate (111 mg, 0.510 mmol, Aldrich) was suspended in toluene (2.5 mL), then cooled to 0° C. A solution of 5-methoxy-8-(4-methyl-piperazin-1-yl)-1,2,3,4-tetrahydro-isoquinoline (122 mg, 0.466 mmol, 11427-50-2) in dichloromethane (1.0 mL) was added dropwise via syringe, and then the reaction mixture was stirred at 0° C. for 45 min. To this was added aniline (2.00 mL, 2.04 g, 21.9 mmol, Aldrich), and the mixture was brought to room temperature. After 15 min, the reaction mixture was diluted with 1:9 methanol/dichloromethane (25 mL) and poured into an equal volume of 20% aqueous potassium carbonate. The phases were separated, and the aqueous portion was extracted with 1:19 methanol/dichloromethane (3×25 mL). The combined extracts were washed with brine (75 mL), dried (sodium sulfate), filtered and concentrated leaving an orange oil. The crude product was purified by fcc to afford 100 mg (40%) white solid. LC/MS (M+1) m/z=536.

### Example 56

### 5-Methoxy-8-(4-methyl-piperazin-1-yl)-3,4-dihydro-1H-isoquinoline-2-carboxylic acid [4-(2-methoxy-phenylsulfamoyl)-phenyl]-amide

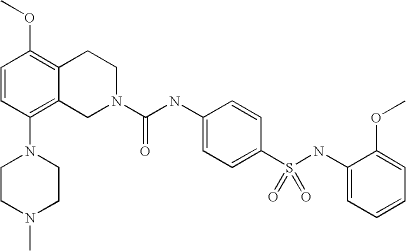

### Example 56

### 5-Methoxy-8-(4-methyl-piperazin-1-yl)-3,4-dihydro-1H-isoquinoline-2-carboxylic acid [4-(2-methoxy-phenylsulfamoyl)-phenyl]-amide

This compound was synthesized from 4-(chlorosulfonyl)phenyl isocyanate (116 mg, 0.533 mmol), 5-methoxy-8-(4-methyl-piperazin-1-yl)-1,2,3,4-tetrahydro-isoquinoline (123 mg, 0.469 mmol, 11427-50-2) and o-anisidine (0.50 mL, 0.546 g, 4.43 mmol, Aldrich), using the same synthetic procedure as demonstrated in Example 11 above. Yield: 120 mg (45%), white solid. LC/MS (M+1) m/z=566.

### Example 57

### 5-Methoxy-8-(4-methyl-piperazin-1-yl)-3,4-dihydro-1H-isoquinoline-2-carboxylic acid [4-(3-methoxy-phenylsulfamoyl)-phenyl]-amide

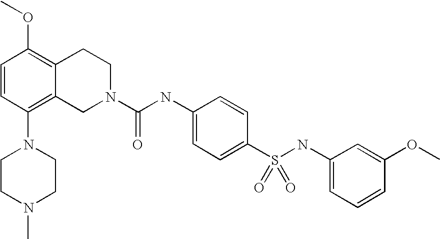

### Example 57

### 5-Methoxy-8-(4-methyl-piperazin-1-yl)-3,4-dihydro-1H-isoquinoline-2-carboxylic acid [4-(3-methoxy-phenylsulfamoyl)-phenyl]-amide

This compound was synthesized from 4-(chlorosulfonyl)phenyl isocyanate (123 mg, 0.565 mmol), 5-methoxy-8-(4-methyl-piperazin-1-yl)-1,2,3,4-tetrahydro-isoquinoline (123 mg, 0.469 mmol, 11427-50-2) and m-anisidine (0.50 mL, 0.548 g, 4.45 mmol, Aldrich), using the same synthetic procedure as demonstrated in Example 11 above. Yield: 80 mg (28%), off-white solid. LC/MS (M+1) m/z=566.

### Example 58

### 5-Methoxy-8-(4-methyl-piperazin-1-yl)-3,4-dihydro-1H-isoquinoline-2-carboxylic acid (4-benzylsulfamoyl-phenyl)-amide

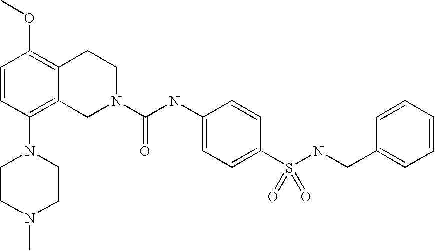

### Example 58

### 5-Methoxy-8-(4-methyl-piperazin-1-yl)-3,4-dihydro-1H-isoquinoline-2-carboxylic acid (4-benzylsulfamoyl-phenyl)-amide

This compound was synthesized from 4-(chlorosulfonyl)phenyl isocyanate (118 mg, 0.542 mmol), 5-methoxy-8-(4-methyl-piperazin-1-yl)-1,2,3,4-tetrahydro-isoquinoline (121 mg, 0.462 mmol, 11427-50-2) and benzylamine (0.50 mL, 0.490 g, 4.58 mmol, Aldrich), using the same synthetic procedure as demonstrated in Example 11 above. Yield: 140 mg (55%), white solid. LC/MS (M+1) m/z=550.

### Example 59

### 5-Methoxy-8-(4-methyl-piperazin-1-yl)-3,4-dihydro-1H-isoquinoline-2-carboxylic acid [4-(2-methoxy-benzylsulfamoyl)-phenyl]-amide

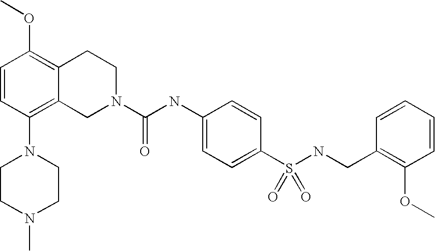

### Example 59

### 5-Methoxy-8-(4-methyl-piperazin-1-yl)-3,4-dihydro-1H-isoquinoline-2-carboxylic acid [4-(2-methoxy-benzylsulfamoyl)-phenyl]-amide

This compound was synthesized from 4-(chlorosulfonyl)phenyl isocyanate (117 mg, 0.538 mmol), 5-methoxy-8-(4-methyl-piperazin-1-yl)-1,2,3,4-tetrahydro-isoquinoline (121 mg, 0.462 mmol, 11427-50-2) and 2-methoxybenzylamine (0.50 mL, 0.525 g, 3.83 mmol, Aldrich), using the same synthetic procedure as demonstrated in Example 11 above. Yield: 134 mg (50%), white solid. LC/MS (M+1) m/z=580.

### Example 60

### 5-Methoxy-8-(4-methyl-piperazin-1-yl)-3,4-dihydro-1H-isoquinoline-2-carboxylic acid [4-(3-methoxy-benzylsulfamoyl)-phenyl]-amide

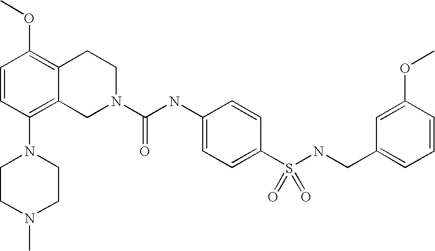

### Example 60

### 5-Methoxy-8-(4-methyl-piperazin-1-yl)-3,4-dihydro-1H-isoquinoline-2-carboxylic acid [4-(3-methoxy-benzylsulfamoyl)-phenyl]-amide

This compound was synthesized from 4-(chlorosulfonyl)phenyl isocyanate (118 mg, 0.542 mmol), 5-methoxy-8-(4-methyl-piperazin-1-yl)-1,2,3,4-tetrahydro-isoquinoline (123 mg, 0.472 mmol, 11427-50-2) and 3-methoxybenzylamine (0.50 mL, 0.54 g, 3.9 mmol, Aldrich), using the same synthetic procedure as demonstrated in Example 11 above. Yield: 130 mg (50%), white solid. LC/MS (M+1) m/z=580.

### Example 61

### 5-Methoxy-8-(4-methyl-piperazin-1-yl)-3,4-dihydro-1H-isoquinoline-2-carboxylic acid [4-(4-methoxy-benzylsulfamoyl)-phenyl]-amide

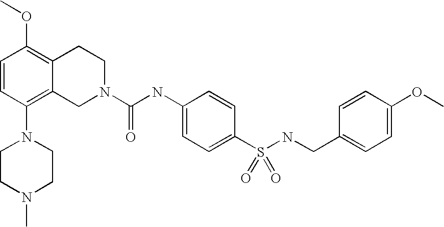

### Example 61

### 5-Methoxy-8-(4-methyl-piperazin-1-yl)-3,4-dihydro-1H-isoquinoline-2-carboxylic acid [4-(4-methoxy-benzylsulfamoyl)-phenyl]-amide

This compound was synthesized from 4-(chlorosulfonyl)phenyl isocyanate (117 mg, 0.538 mmol), 5-methoxy-8-(4-methyl-piperazin-1-yl)-1,2,3,4-tetrahydro-isoquinoline (123 mg, 0.472 mmol, 11427-50-2) and 4-methoxybenzylamine (0.50 mL, 0.52 g, 3.8 mmol, Aldrich), using the same synthetic procedure as demonstrated in Example 11 above. Yield: 122 mg (44%), white foam. LC/MS (M+1) m/z=580.

### Example 62

### 5-Methoxy-8-(4-methyl-piperazin-1-yl)-3,4-dihydro-1H-isoquinoline-2-carboxylic acid (4-propylsulfamoyl-phenyl)-amide

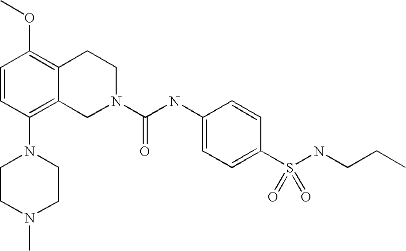

### Example 62

### 5-Methoxy-8-(4-methyl-piperazin-1-yl)-3,4-dihydro-1H-isoquinoline-2-carboxylic acid (4-propylsulfamoyl-phenyl)-amide

This compound was synthesized from 4-(chlorosulfonyl)phenyl isocyanate (117 mg, 0.538 mmol), 5-methoxy-8-(4-methyl-piperazin-1-yl)-1,2,3,4-tetrahydro-isoquinoline (121 mg, 0.462 mmol, 11427-50-2) and propylamine (0.500 mL, 0.359 g, 6.08 mmol, Acros), using the same synthetic procedure as demonstrated in Example 11 above. Yield: 115 mg (53%), white solid. LC/MS (M+1) m/z 502.

### Example 63

### 5-Methoxy-8-(4-methyl-piperazin-1-yl)-3,4-dihydro-1H-isoquinoline-2-carboxylic acid (4-isopropylsulfamoyl-phenyl)-amide

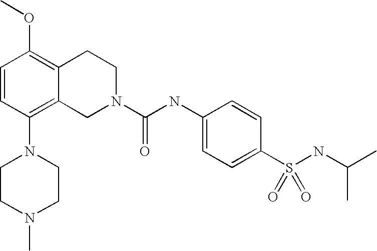

### Example 63

### 5-Methoxy-8-(4-methyl-piperazin-1-yl)-3,4-dihydro-1H-isoquinoline-2-carboxylic acid (4-isopropylsulfamoyl-phenyl)-amide

This compound was synthesized from 4-(chlorosulfonyl)phenyl isocyanate (1117 mg, 0.538 mmol), 5-methoxy-8-(4-methyl-piperazin-1-yl)-1,2,3,4-tetrahydro-isoquinoline (121 mg, 0.462 mmol, 11427-50-2) and isopropylamine (0.500 mL, 0.347 g, 5.87 mmol, Aldrich), using the same synthetic procedure as demonstrated in Example 11 above. Yield: 140 mg (64%), white solid. LC/MS (M+1) m/z=502.

### Example 64

### 5-Methoxy-8-(4-methyl-piperazin-1-yl)-3,4-dihydro-1H-isoquinoline-2-carboxylic acid (4-cyclopropylsulfamoyl-phenyl)-amide

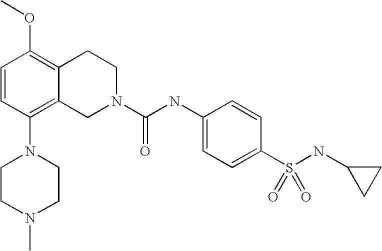

### Example 64

### 5-Methoxy-8-(4-methyl-piperazin-1-yl)-3,4-dihydro-1H-isoquinoline-2-carboxylic acid (4-cyclopropylsulfamoyl-phenyl)-amide

This compound was synthesized from 4-(chlorosulfonyl)phenyl isocyanate (115 mg, 0.528 mmol), 5-methoxy-8-(4-methyl-piperazin-1-yl)-1,2,3,4-tetrahydro-isoquinoline (121 mg, 0.462 mmol, 11427-50-2) and cyclopropylamine (0.500 mL, 0.412 g, 7.21 mmol, Aldrich), using the same synthetic procedure as demonstrated in Example 11 above. Yield: 110 mg (50%), white solid. LC/MS (M+1) m/z=500.

### Example 65

### 5-Methoxy-8-(4-methyl-piperazin-1-yl)-3,4-dihydro-1H-isoquinoline-2-carboxylic acid (4-tert-butylsulfamoyl-phenyl)-amide

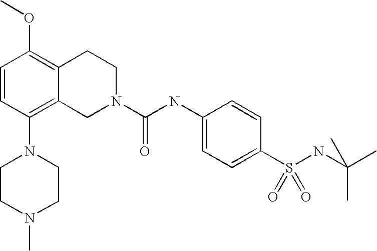

### Example 65

### 5-Methoxy-8-(4-methyl-piperazin-1-yl)-3,4-dihydro-1H-isoquinoline-2-carboxylic acid (4-tert-butylsulfamoyl-phenyl)-amide

This compound was synthesized from 4-(chlorosulfonyl)phenyl isocyanate (118 mg, 0.542 mmol), 5-methoxy-8-(4-methyl-piperazin-1-yl)-1,2,3,4-tetrahydro-isoquinoline (121 mg, 0.462 mmol, 11427-50-2) and tert-butylamine (0.500 mL, 0.348 g, 4.76 mmol, Aldrich), using the same synthetic procedure as demonstrated in Example 11 above. Yield: 120 mg (53%), white solid. LC/MS (M+1) m/z=516.

### Example 66

### 5-Methoxy-8-(4-methyl-piperazin-1-yl)-3,4-dihydro-1H-isoquinoline-2-carboxylic acid (4-methylsulfamoyl-phenyl)-amide

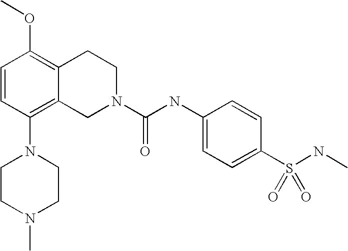

### Example 66

### 5-Methoxy-8-(4-methyl-piperazin-1-yl)-3,4-dihydro-1H-isoquinoline-2-carboxylic acid (4-methylsulfamoyl-phenyl)-amide

This compound was synthesized from 4-(chlorosulfonyl)phenyl isocyanate (116 mg, 0.533 mmol), 5-methoxy-8-(4-methyl-piperazin-1-yl)-1,2,3,4-tetrahydro-isoquinoline (121 mg, 0.464 mmol, 11427-50-2) and methylamine (2M in THF, 2.5 mL, 5.0 mmol, Aldrich), using the same synthetic procedure as demonstrated in Example 11 above. Yield: 100 mg (47%), white solid. LC/MS (M+1) m/z=474.

### Example 67

### 5-Methoxy-8-(4-methyl-piperazin-1-yl)-3,4-dihydro-1H-isoquinoline-2-carboxylic acid (4-ethylsulfamoyl-phenyl)-amide

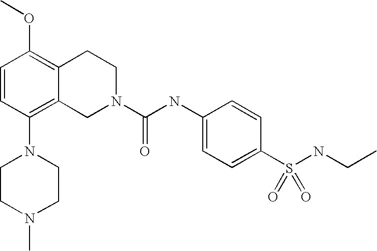

### Example 67

### 5-Methoxy-8-(4-methyl-piperazin-1-yl)-3,4-dihydro-1H-isoquinoline-2-carboxylic acid (4-ethylsulfamoyl-phenyl)-amide

This compound was synthesized from 4-(chlorosulfonyl)phenyl isocyanate (119 mg, 0.547 mmol), 5-methoxy-8-(4-methyl-piperazin-1-yl)-1,2,3,4-tetrahydro-isoquinoline (121 mg, 0.464 mmol, 11427-50-2) and ethylamine (2.0M in THF, 2.5 mL, 5.0 mmol, Aldrich), using the same synthetic procedure as demonstrated in Example 11 above. Yield: 120 mg (55%), white solid. LC/MS (M+1) m/z=488.

### Example 68

### 5-Methoxy-8-(4-methyl-piperazin-1-yl)-3,4-dihydro-1H-isoquinoline-2-carboxylic acid (4-cyclobutylsulfamoyl-phenyl)-amide

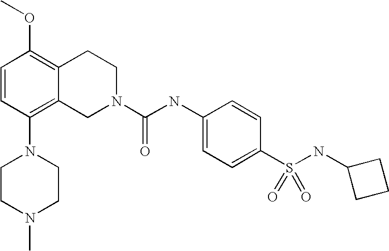

### Example 68

### 5-Methoxy-8-(4-methyl-piperazin-1-yl)-3,4-dihydro-1H-isoquinoline-2-carboxylic acid (4-cyclobutylsulfamoyl-phenyl)-amide

This compound was synthesized from 4-(chlorosulfonyl)phenyl isocyanate (116 mg, 0.533 mmol), 5-methoxy-8-(4-methyl-piperazin-1-yl)-1,2,3,4-tetrahydro-isoquinoline (121 mg, 0.464 mmol, 11427-50-2) and cyclobutylamine (0.50 mL, 0.416 g, 5.86 mmol, Aldrich), using the same synthetic procedure as demonstrated in Example 11 above. Yield: 130 mg (56%), white solid. LC/MS (M+1) m/z=514.

### Example 69

### 5-Methoxy-8-(4-methyl-piperazin-1-yl)-3,4-dihydro-1H-isoquinoline-2-carboxylic acid [4-(thiazol-2-ylsulfamoyl)-phenyl]-amide

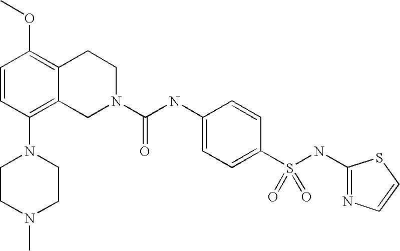

### Example 69

### 5-Methoxy-8-(4-methyl-piperazin-1-yl)-3,4-dihydro-1H-isoquinoline-2-carboxylic acid [4-(thiazol-2-ylsulfamoyl)-phenyl]-amide

4-(Chlorosulfonyl)phenyl isocyanate (127 mg, 0.583 mmol, Aldrich) was suspended in toluene (2.5 mL), cooled to 0° C., and treated dropwise with a solution of 5-methoxy-8-(4-methyl-piperazin-1-yl)-1,2,3,4-tetrahydro-isoquinoline (132 mg, 0.505 mmol) in dichloromethane (2.0 mL). After 30 min a solution of 2-aminothiazole (394 mg, 3.93 mmol, Aldrich) in DMF (1.5 mL) was added to the reaction along with a catalytic amount of DMAP. The mixture was brought to room temperature, stirred 3 h, and then diluted with 1:19 methanol/dichloromethane (25 mL) and poured into 10% aqueous sodium carbonate (25 mL). The phases were separated, and the aqueous phase was extracted with dichloromethane (2×25 mL). The combined extracts were dried (sodium sulfate), filtered, and concentrated leaving a yellow oil. The oil was triturated with ether, the resulting solid was filtered, and the crude product was purified by prep HPLC. Only fractions containing pure product were combined, leaving 15 mg (5%) yellow semi-solid.

LC/MS (M+1) m/z=543.

### Example 70

### 5-Methoxy-8-(4-methyl-piperazin-1-yl)-3,4-dihydro-1H-isoquinoline-2-carboxylic acid (4-acetylsulfamoyl-phenyl)-amide

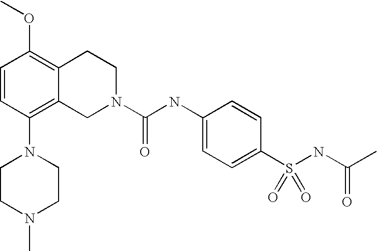

### Example 70

### 5-Methoxy-8-(4-methyl-piperazin-1-yl)-3,4-dihydro-1H-isoquinoline-2-carboxylic acid (4-acetylsulfamoyl-phenyl)-amide

5-Methoxy-8-(4-methyl-piperazin-1-yl)-3,4-dihydro-1H-isoquinoline-2-carboxylic acid (4-sulfamoyl-phenyl)-amide (98.6 mg, 0.214 mmol, 11837-68-1, as prepared in Example 28) was suspended in dichloromethane (3.0 mL), treated with triethylamine (0.313 mL, 0.227 g, 2.25 mmol) and acetyl chloride (0.199 ml, 0.220 g, 2.80 mmol). The reaction mixture was stirred at room temperature for 24 h, whereupon reaction was complete. The reaction mixture was treated with 1:1 acetonitrile/0.1% trifluoroacetic acid in water (3 mL) and allowed to stand overnight. The dichloromethane was removed under reduced pressure, and the remaining solution was filtered and purified by prep HPLC. Fractions containing the desired product were combined, concentrated, and the residue was triturated with ether. The resulting precipitate was filtered, and the product was collected as the trifluoroacetic acid salt. Yield: 45.0 mg (34%). LC/MS (M+1) m/z=502.

### Example 71

### 5-Methoxy-8-(4-methyl-piperazin-1-yl)-3,4-dihydro-1H-isoquinoline-2-carboxylic acid (4-butyrylsulfamoyl-phenyl)-amide

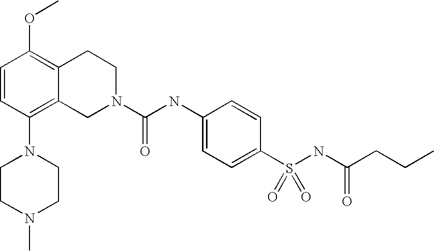

### Example 71a

### 5-Methoxy-8-(4-methyl-piperazin-1-yl)-3,4-dihydro-1H-isoquinoline-2-carboxylic acid (4-sulfamoyl-phenyl)-amide

4-(Chlorosulfonyl)phenyl isocyanate (0.479 g, 2.20 mmol, Aldrich) was suspended in toluene (10 mL), cooled to 0° C., and treated dropwise with a solution of 5-methoxy-8-(4-methyl-piperazin-1-yl)-1,2,3,4-tetrahydro-isoquinoline (0.5236 g, 2.003 mmol) in dichloromethane (10 mL). After 15 min a solution of ammonia (2.0 M in isopropyl alcohol, 8.0 mL, 16 mmol) was added, and the mixture was brought to room temperature. After 2 h the reaction was quenched by addition of 10% aqueous sodium carbonate (100 mL). The biphasic mixture was separated, and the aqueous phase was extracted with dichloromethane (2×125 mL). The combined extracts were dried (sodium sulfate), filtered, and concentrated leaving a pale yellow solid. The crude product was purified by fcc, and pure product fractions were combined and concentrated leaving 201.2 mg (21%) white solid. LC/MS (M+1) m/z=460.

### Example 71

### 5-Methoxy-8-(4-methyl-piperazin-1-yl)-3,4-dihydro-1H-isoquinoline-2-carboxylic acid (4-butyrylsulfamoyl-phenyl)-amide

5-Methoxy-8-(4-methyl-piperazin-1-yl)-3,4-dihydro-1H-isoquinoline-2-carboxylic acid (4-sulfamoyl-phenyl)-amide (0.248 g, 0.540 mmol, prepared as in Example 28) was suspended in dichloromethane (6 mL), treated with triethylamine (0.167 mL, 0.121 g, 1.19 mmol) and butyryl chloride (0.112 mL, 0.115 g, 1.08 mmol), and the mixture was heated to reflux 16 h. The reaction mixture was diluted with 1:9 methanol/chloroform (70 mL) and poured into 4% aqueous sodium bicarbonate (40 mL). The phases were separated, and the organic portion was dried (sodium sulfate), filtered and concentrated leaving a gummy semi-solid. The residue was triturated with isopropyl alcohol and ether; the solid was filtered and dried under high vacuum, leaving 118 mg (41%) white solid.

LC/MS (M+1) m/z-530. mp=185-190° C.

### Example 72

### 5-Methoxy-8-(4-methyl-piperazin-1-yl)-3,4-dihydro-1H-isoquinoline-2-carboxylic acid [4-(methyl-phenyl-sulfamoyl)-phenyl]-amide

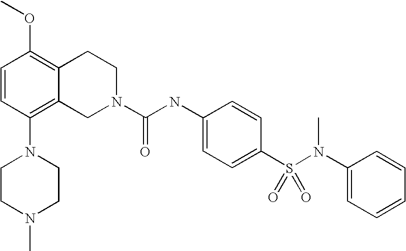

### Example 72a

### 4-{[5-Methoxy-8-(4-methyl-piperazin-1-yl)-3,4-dihydro-1H-isoquinoline-2-carbonyl]-amino}-benzenesulfonyl chloride

4-(Chlorosulfonyl)phenyl isocyanate (7.78 g, 35.7 mmol, Aldrich) was suspended in toluene (170 mL), cooled to 0° C., and treated dropwise with a solution of 5-methoxy-8-(4-methyl-piperazin-1-yl)-1,2,3,4-tetrahydro-isoquinoline (8.49 g, 32.5 mmol) in dichloromethane (180 mL). The reaction mixture was kept at 0° C. for 15 min following the addition, the entire mixture was diluted with ether, and the solid product was filtered leaving 11.51 g (74%) white solid. LC/MS (M+1) m/z=479.

### Example 72

### 5-Methoxy-8-(4-methyl-piperazin-1-yl)-3,4-dihydro-1H-isoquinoline-2-carboxylic acid [4-(methyl-phenyl-sulfamoyl)-phenyl]-amide

A solution of N-methylaniline (0.0250 mL, 24.7 mg, 0.231 mmol, Aldrich) in pyridine (2.0 mL, anhydrous) was treated with 4-{[5-methoxy-8-(4-methyl-piperazin-1-yl)-3,4-dihydro-1H-isoquinoline-2-carbonyl]-amino}-benzenesulfonyl chloride (0.108 g, 0.225 mmol, 11837-102-1, as prepared in Example 30), and the bright yellow-orange solution was heated to 60° C. for 45 min. The reaction mixture was cooled to room temperature and diluted with 1:9 methanol/chloroform (50 mL). The solution was poured into aqueous saturated sodium bicarbonate (50 mL), and the phases were separated. The aqueous phase was extracted with 1:19 methanol/chloroform (50 mL), and the combined organic portions were dried (sodium sulfate), filtered and concentrated leaving a bright yellow oil. The crude product was purified fcc on 5 g silica gel. Pure product fractions were combined and concentrated leaving 51 mg (41%) orange foam. LC/MS (M+1) m/z=550

### Example 73

### 5-Methoxy-8-(4-methyl-piperazin-1-yl)-3,4-dihydro-1H-isoquinoline-2-carboxylic acid [4-(acetyl-methyl-sulfamoyl)-phenyl]-amide

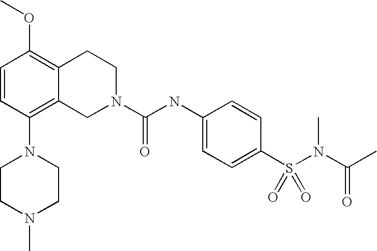

### Example 73a

### 5-Methoxy-8-(4-methyl-piperazin-1-yl)-3,4-dihydro-1H-isoquinoline-2-carboxylic acid (4-methylsulfamoyl-phenyl)-amide

4-{[5-Methoxy-8-(4-methyl-piperazin-1-yl)-3,4-dihydro-1H-isoquinoline-2-carbonyl]-amino}-benzenesulfonyl chloride (0.4924 g, 1.028 mmol, 11837-102-1, as prepared in Example 30 above) was added to a solution of methylamine (2.0M in THF, 5.0 mL, 10 mmol) at room temperature and stirred for 30 min. The reaction mixture was quenched by addition of saturated aqueous sodium bicarbonate (2 mL), diluted with 1:19 methanol/chloroform (20 mL), and poured into water (20 mL). The phases were separated, and the aqueous portion was extracted with 1:19 methanol/chloroform (20 mL). The combined organics were dried (sodium sulfate), filtered and concentrated leaving a semi-solid, which was triturated with ether. The solid residue was purified by fcc to afford 0.224 g (46%) white solid. LC/MS (M+1) m/z=474.

### Example 73

### 5-Methoxy-8-(4-methyl-piperazin-1-yl)-3,4-dihydro-1H-isoquinoline-2-carboxylic acid [4-(acetyl-methyl-sulfamoyl)-phenyl]-amide

5-Methoxy-8-(4-methyl-piperazin-1-yl)-3,4-dihydro-1H-isoquinoline-2-carboxylic acid (4-methylsulfamoyl-phenyl)-amide (0.1011 g, 0.2135 mmol, 1.1837-111-1, as prepared in Example 32) was suspended in dichloromethane (2 mL), and treated with triethylamine (0.150 mL, 0.109 g, 1.08 mmol) and acetyl chloride (0.070 mL, 77.3 mg, 0.984 mmol). The reaction mixture was warmed to 40° C. for 2.5 h; the solution was then cooled to room temperature and allowed to stir 16 h. The reaction was quenched with 4% aqueous sodium bicarbonate (20 mL) and poured into 1:9 methanol/chloroform (25 mL). The phases were separated, and the aqueous portion was extracted with chloroform (25 mL). The combined organic portions were dried (sodium sulfate), filtered and concentrated leaving a yellow semi-solid. The crude product was purified by fcc on 5 g silica gel to afford 80.1 mg (73%) yellow-white solid.

LC/MS (M+1) m/z=516.

### Example 74

### 1-[5-Methoxy-8-(4-methyl-piperazin-1-yl)-3,4-dihydro-1H-isoquinolin-2-yl]-2-(4-morpholin-4-yl-phenyl)-ethanone

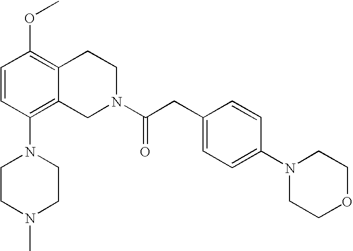

### Example 74a

### 2-(4-Bromo-phenyl)-1-[5-methoxy-8-(4-methyl-piperazin-1-yl)-3,4-dihydro-1H-isoquinolin-2-yl]-ethanone

A solution of 4-bromoacetic acid ((0.215 g, 1 mmole) in 10 mL of dichloromethane was cooled to 0 C and treated with a drop of dimethylformamide followed by oxalylchloride (0.254 g, 0.174 mL). Upon stirring at the room temperature for 3 h, the reaction mixture was concentrated under reduced pressure, dissolved in 10 mL of dichloromethane, cooled to 0 C and added to 5-Methoxy-8-(4-methyl-piperazin-1-yl)-1,2,3,4-tetrahydro-isoquinoline (Example 1d) (0.25 g, 0.94 mmole). The resulting reaction mixture was treated with triethylamine (0.28 mL, 2 mmole) and stirred at room temperature for 16 h. Upon diluting with dichloromethane and washing with potassium carbonate solution the organic layer was dried over potassium carbonate and concentrated under reduced pressure to afford the desired product (0.42 g); LC MS (M+1)) m/e 458.

### Example 74

### 1-[5-Methoxy-8-(4-methyl-piperazin-1-yl)-3,4-dihydro-1H-isoquinolin-2-yl]-2-(4-morpholin-4-yl-phenyl)-ethanone

A suspension of BINAP (6 mg, 0.025 mmol) and Pd₂(dba)₃ (9 mg, 0.01 mmole) in toluene (10 mL) was treated with a solution of 2-(4-Bromo-phenyl)-1-[5-methoxy-8-(4-methyl-piperazin-1-yl)-3,4-dihydro-1H-isoquinolin-2-yl]-ethanone (Example 74a) (0.42 g 0.92 mmole) in toluene (25 mL) followed by morpholine (0.113 g, 1.3 mmole). After 5 minutes cesium carbonate (065.1 g, 2 mole) was added and the reaction mixture was heated to 110 for 16 h. Same amount of BINAP,Pd₂(dba)₃, cesium carbonate and morpholine were added as above and the reaction mixture was heated to 110 C for additional 16 h. At the end of this period, the reaction mixture was cooled to room temperature, diluted with dichloromethane and washed with an aqueous solution of potassium carbonate. Upon drying the organic layer over anhydrous potassium carbonate and concentrating under reduced pressure the product was purified by column chromatography over 40 g of silica gel. Elution with dichloromethane containing methanol and ammonia afforded the desired material (0.19 g). This material was further purified by preparative HPLC. The fractions contain the desired product were combined lyophilized to afford the desired material (82 mg); LC/MS (M+1) m/z 465.

### Example 75

### 2-(4-Dimethylamino-phenyl)-1-[5-methoxy-8-(4-methyl-piperazin-1-yl)-3,4-dihydro-1H-isoquinolin-2-yl]-ethanone

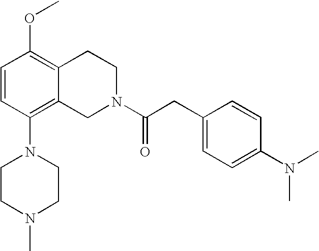

### Example 75

### 2-(4-Dimethylamino-phenyl)-1-[5-methoxy-8-(4-methyl-piperazin-1-yl)-3,4-dihydro-1H-isoquinolin-2-yl]-ethanone

A solution of 4-dimethylaminophenylacetic acid (1 mmole, 0.179 g) in 10 mL of dichloromethane was treated with 1 drop of dimethylformamide followed by oxalylchloride (2 mmole, 0.17 ml). Upon stirring for 16 h the reaction mixture was concentrated under reduced pressure and dissolved in 15 mL of dichloromethane. The resulting solution was treated with 5-Methoxy-8-(4-methyl-piperazin-1-yl)-1,2,3,4-tetrahydro-isoquinoline (Example 1d) followed by triethylamine and the reaction mixture was stirred for 16 h and diluted with dichloromethane. Washing with a solution of potassium carbonate, drying over anhydrous potassium carbonate and concentration under reduced pressure afforded the crude product which was purified by fcc to afford 0.257 g; LCMS (M+1) m/z 423.

### Example 76

### 1-[5-Methoxy-8-(4-methyl-piperazin-1-yl)-3,4-dihydro-1H-isoquinolin-2-yl]-2-(3-morpholin-4-yl-phenyl)-ethanone

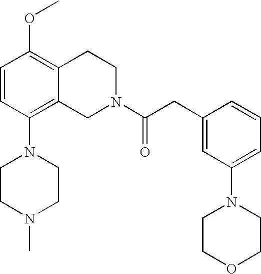

### Example 76a

### 2-(3-Bromo-phenyl)-1-[5-methoxy-8-(4-methyl-piperazin-1-yl)-3,4-dihydro-1H-isoquinolin-2-yl]-ethanone

The compound was prepared from 3-bromophenylacetic acid using a method similar to the one described for Example 74. (0.25 g); LCMS (M+1) m/z 458.

### Example 76

### 1-[5-Methoxy-8-(4-methyl-piperazin-1-yl)-3,4-dihydro-1H-isoquinolin-2-yl]-2-(3-morpholin-4-yl-phenyl)-ethanone

A suspension of sodium-tert-butoxide (0.079 g, 0.83 mmole) in morpholine (0.062 mmole; 0.062 mL) was treated with a solution of 2-(3-Bromo-phenyl)-1-[5-methoxy-8-(4-methyl-piperazin-1-yl)-3,4-dihydro-1H-isoquinolin-2-yl]-ethanone (Example 76a) (0.25 g, 0.55 mmole) in 1 mL of toluene. To this mixture was added to a suspension of Pd₂(dba)₃ (0.025 g, 0.028 mmole) and BINAP (0.052 g, 0.084 mmole) in 5 mL of toluene. The resulting mixture was heated to 100 C for 16 h under nitrogen. At the end of this period the reaction mixture was purified by fcc to afford the desired material (0.112 g); LCMS (M+1) m/z 465.

### Example 77

### 1-[5-Methoxy-8-(4-methyl-piperazin-1-yl)-3,4-dihydro-1H-isoquinolin-2-yl]-2-(4-piperidin-1-yl-phenyl)-ethanone

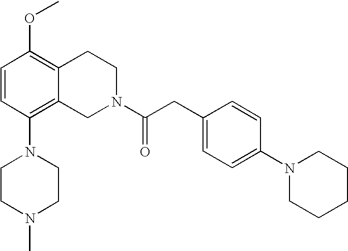

### Example 77

### 1-[5-Methoxy-8-(4-methyl-piperazin-1-yl)-3,4-dihydro-1H-isoquinolin-2-yl]-2-(4-piperidin-1-yl-phenyl)-ethanone

A solution of 2-(4-Bromo-phenyl)-1-[5-methoxy-8-(4-methyl-piperazin-1-yl)-3,4-dihydro-1H-isoquinolin-2-yl]-ethanone (Example 74a) (45.6 mg, 0.1 mmole) and 2(di-t-butylphosphino)biphenyl (0.005 mmole, 1.5 mg) and piperidine (0.012 mL, 0.12 mmole) in 2 mL of toluene was treated with sodium tert-butoxide (0.0135 g, 0.14 mmole) and Pd₂(dba)₃ (0.002 mmole, 0.0018 g). The resulting mixture was heated to 100 C for 16 h. The product obtained from three similar reactions were combined and purified preparative HPLC to afford the desired material (30 mg); LCMS (M+) m/z 464.

### Example 78

### 1-[5-Methoxy-8-(4-methyl-piperazin-1-yl)-3,4-dihydro-1H-isoquinolin-2-yl]-2-[4-(4-methyl-piperazin-1-yl)-phenyl]-ethanone

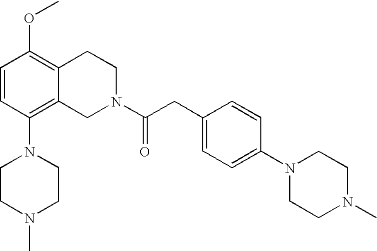

### Example 78

### 1-[5-Methoxy-8-(4-methyl-piperazin-1-yl)-3,4-dihydro-1H-isoquinolin-2-yl]-2-[4-(4-methyl-piperazin-1-yl)-phenyl]-ethanone

A solution of 2-(4-Bromo-phenyl)-1-[5-methoxy-8-(4-methyl-piperazin-1-yl)-3,4-dihydro-1H-isoquinolin-2-yl]-ethanone (Example 74a) (0.151 g, 0.33 mmole) in $ mL of toluene was treated with N-methylpiperizine (0.4 mmole, 40 mg), sodium tert-butoxide (0.46 mmole, 44 mg), 2(di-tertbutylphosphino)biphenyl (5 mg, 0.0165 mmole) and Pd₂(dba)₃ (0.0066 mmole, 6 mg). The resulting reaction mixture was heated to 100 C for 6 h and purified by prep

LCMS to afford the desired product (40 mg); LCMS (M+1) m/z 478.6.

### Example 79

### 1-[5-Methoxy-8-(4-methyl-piperazin-1-yl)-3,4-dihydro-1H-isoquinolin-2-yl]-2-[4-(4-propyl-piperidin-1-yl)-phenyl]-ethanone

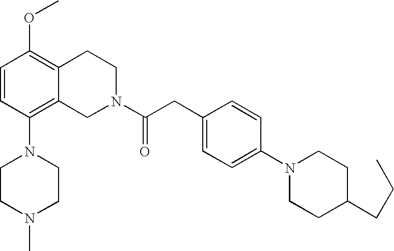

### Example 79

### 1-[5-Methoxy-8-(4-methyl-piperazin-1-yl)-3,4-dihydro-1H-isoquinolin-2-yl]-2-[4-(4-propyl-piperidin-1-yl)-phenyl]-ethanone

This compound was prepared by method similar to the one described for Example 78 except that 4-propypiperidine (0.0508 g) was used instead of N-methylpiperizine; LCMS (M+1) m/z 505.6.

### Example 80

### 2-{4-[4-(2-Methoxy-ethyl)-piperidin-1-yl]-phenyl}-1-[5-methoxy-8-(4-methyl-piperazin-1-yl)-3,4-dihydro-1H-isoquinolin-2-yl]-ethanone

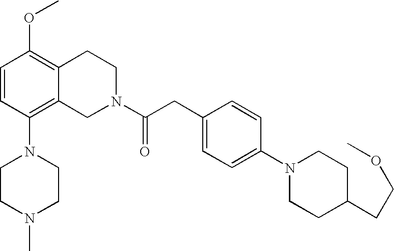

### Example 80

### 2-{4-[4-(2-Methoxy-ethyl)-piperidin-1-yl]-phenyl}-1-[5-methoxy-8-(4-methyl-piperazin-1-yl)-3,4-dihydro-1H-isoquinolin-2-yl]-ethanone

This compound was prepared by method similar to the one described for Example 78 except that 4-methoxyethylpiperidine (0.057 g) was used instead of N-methylpiperizine; LCMS (M+1) m/z 522.6.

### Example 81

### 1-[5-Methoxy-8-(4-methyl-piperazin-1-yl)-3,4-dihydro-1H-isoquinolin-2-yl]-2-[4-(4-methyl-piperidin-1-yl)-phenyl]-ethanone

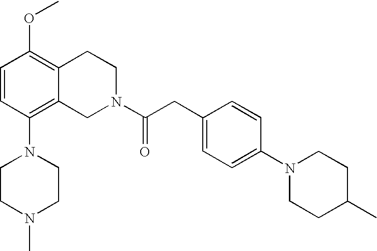

### Example 81

### 1-[5-Methoxy-8-(4-methyl-piperazin-1-yl)-3,4-dihydro-1H-isoquinolin-2-yl]-2-[4-(4-methyl-piperidin-1-yl)-phenyl]-ethanone

This compound was prepared by method similar to the one described for Example 78 except that 4-methylpiperidine (0.039 g) was used instead of N-methylpiperizine. LCMS (M+1) m/z 477.6

### Example 82

### 2-[4-(4-Hydroxy-piperidin-1-yl)-phenyl]-1-[5-methoxy-8-(4-methyl-piperazin-1-yl)-3,4-dihydro-1H-isoquinolin-2-yl]-ethanone

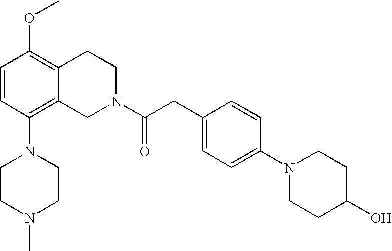

### Example 82

### 2-[4-(4-Hydroxy-piperidin-1-yl)-phenyl]-1-[5-methoxy-8-(4-methyl-piperazin-1-yl)-3,4-dihydro-1H-isoquinolin-2-yl]-ethanone

This compound was prepared by method similar to the one described for Example 78 except that 4-hydroxypiperidine (0.033 g) was used instead of N-methylpiperizine. LCMS (M+1) m/z 479.6

### Example 83

### 2-{4-[4-(2-Hydroxy-ethyl)-piperazin-1-yl]-phenyl}-1-[5-methoxy-8-(4-methyl-piperazin-1-yl)-3,4-dihydro-1H-isoquinolin-2-yl]-ethanone

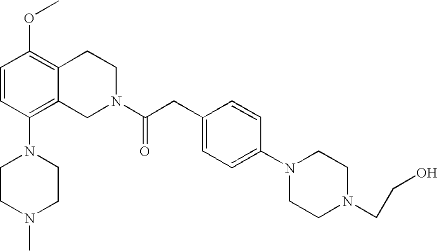

### Example 83

### 2-{4-[4-(2-Hydroxy-ethyl)-piperazin-1-yl]-phenyl}-1-[5-methoxy-8-(4-methyl-piperazin-1-yl)-3,4-dihydro-1H-isoquinolin-2-yl]-ethanone

This compound was prepared by method similar to the one described for Example 78 except that beta-hydroxyethylpiperizine (0.033 g) was used instead of N-methylpiperizine. LCMS (M+1) m/z 508.

### Example 84

### 2-(4-Amino-phenyl)-1-[5-methoxy-8-(4-methyl-piperazin-1-yl)-3,4-dihydro-1H-isoquinolin-2-yl]-ethanone

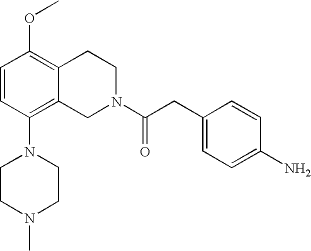

### Example 84

### 2-(4-Amino-phenyl)-1-[5-methoxy-8-(4-methyl-piperazin-1-yl)-3,4-dihydro-1H-isoquinolin-2-yl]-ethanone

A solution of 1-[5-Methoxy-8-(4-methyl-piperazin-1-yl)-3,4-dihydro-1H-isoquinolin-2-yl]-2-(4-nitro-phenyl)-ethanone (Example 84a) (1.29 mmole, 0.546 g) in 50 mL of ethanol was treated with concentrated hydrochloric acid (0.5 mL) followed by 10% Pd/C (75 mg) and hydrogenated at 40 psi of hydrogen for 16 h. At the end of this period the reaction mixture was filtered through diatomaceous earth and concentrated under reduced pressure to afford the crude product which was purified by prep HPLC to afford the desired product (80 mg);

LCMS (M+1) m/z 395.

### Example 84a

A solution of 5-Methoxy-8-(4-methyl-piperazin-1-yl)-1,2,3,4-tetrahydro-isoquinoline (Example 1d) (0.262 g, 1 mmole) in 20 mL of dichloromethane was treated with triethylamine (0.28 mL, 2 mmole), 4-nitrophenylacetic acid (0.199 g, 1.1 mmole) and HATU (0.38 g, 1 mmole). Upon stirring for 16 h the reaction mixture was diluted with dichloromethane and washed with potassium carbonate solution. The organic layer was dried over potassium carbonate and concentrated under reduced pressure to afford the desired material which was used in the next step without further purification; LCMS(M+1) m/z 425.

### Example 85

### 2-(4-Isopropyl-phenoxy)-1-[5-methoxy-8-(4-methyl-piperazin-1-yl)-3,4-dihydro-1H-isoquinolin-2-yl]-ethanone

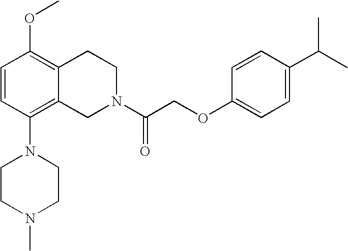

### Example 85

### 2-(4-Isopropyl-phenoxy)-1-[5-methoxy-8-(4-methyl-piperazin-1-yl)-3,4-dihydro-1H-isoquinolin-2-yl]-ethanone

A solution of 5-Methoxy-8-(4-methyl-piperazin-1-yl)-1,2,3,4-tetrahydro-isoquinoline (Example 1d) (0.262 g, 1 mmole) and 4-isopropylphenoxyacetic acid (0.194 g, 1 mmole) in 10 mL of dichloromethane was treated with triethylamine (0.28 mL, 2 mmole) followed by HATU (0.380 g, 1 mmole). Upon stirring for 16 h the reaction mixture was diluted with dichloromethane, washed with potassium carbonate solution and dried over potassium carbonate. Concentration under reduced pressure afforded the desired product. (0.405 g);

LCMS (M+1) m/z 438.6.

### Example 86

### 2-[4-(4-Benzyl-piperazin-1-yl)-phenyl]-1-[5-methoxy-8-(4-methyl-piperazin-1-yl)-3,4-dihydro-1H-isoquinolin-2-yl]-ethanone

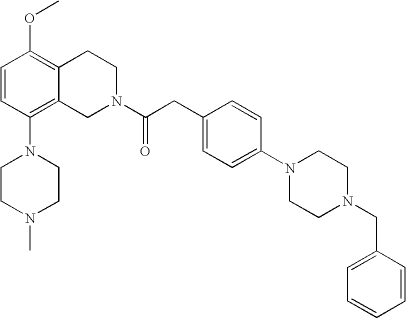

### Example 86

### 2-[4-(4-Benzyl-piperazin-1-yl)-phenyl)-1-[5-methoxy-8-(4-methyl-piperazin-1-yl)-3,4-dihydro-1H-isoquinolin-2-yl]-ethanone

This compound was prepared by method similar to the one described for Example 74 except that N-benzylpiperizine (0.070 g) was used instead of N-methylpiperizine to afford 0.036 g of the desired material; LCMS (M+1) m/z 554.6.

### Example 87

### 2-(4-Isopropyl-phenyl)-1-[5-methoxy-8-(4-methyl-piperazin-1-yl)-3,4-dihydro-1H-isoquinolin-2-yl]-ethanone

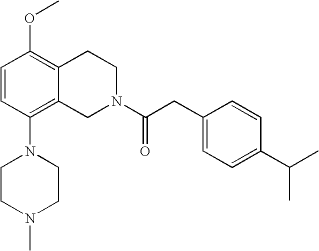

### Example 87

### 2-(4-Isopropyl-phenyl)-1-[5-methoxy-8-(4-methyl-piperazin-1-yl)-3,4-dihydro-1H-isoquinolin-2-yl]-ethanone

A solution of 5-Methoxy-8-(4-methyl-piperazin-1-yl)-1,2,3,4-tetrahydro-isoquinoline (Example 1d) (0.131 g, 0.5 mmole) in 10 mL of dichloromethane was treated with 4-isopropylphenylacetic acid (0.089 g, 0.5 mmole) followed by triethylamine (0.14 mL, 1 mmole) and HATU (0.19 g, 0.5 mmole). Upon stirring for 16 h the reaction mixture was diluted with dichloromethane and washed with potassium carbonate solution. Drying over potassium carbonate and concentration under reduced pressure afforded the desired material (220 mg); LCMS (M+1) m/z 422.2.

### Example 88

### 5-Methoxy-8-(4-methyl-piperazin-1-yl)-3,4-dihydro-1H-isoquinoline-2-carboxylic acid (4-thiomorpholin-4-yl-phenyl)-amide

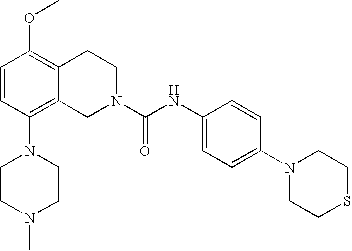

### Example 88

### 5-Methoxy-8-(4-methyl-piperazin-1-yl)-3,4-dihydro-1H-isoquinoline-2-carboxylic acid (4-thiomorpholin-4-yl-phenyl)-amide

A solution of 4-thiomorpholinoaniline (0.087 g) in 2 mL of dichloromethane was treated with carbonyldiimidazole (0.081 g, 0.5 mmole) and upon stirring for 15 min. the reaction mixture was treated with 5-Methoxy-8-(4-methyl-piperazin-1-yl)-1,2,3,4-tetrahydro-isoquinoline (Example 1d) (0.131 g, 0.5 mmole) followed by triethylamine (0.14 mL, 1 mmole). Upon stirring for 16 h the reaction mixture was purified by fcc to afford the desired product. (0.194 g); LCMS (M+D) m/z 483.

### Example 89

### 4-Amino-N-(4-{2-[5-methoxy-8-(4-methyl-piperazin-1-yl)-3,4-dihydro-1H-isoquinolin-2-yl]-2-oxo-ethyl}-phenyl)-butyramide

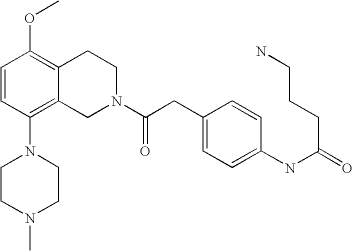

### Example 89a

### [3-(4-{2-[5-Methoxy-8-(4-methyl-piperazin-1-yl)-3,4-dihydro-1H-isoquinolin-2-yl]-2-oxo-ethyl}-phenylcarbamoyl)-propyl]-carbamic acid tert-butyl ester

A solution of 2-(4-Amino-phenyl)-1-[5-methoxy-8-(4-methyl-piperazin-1-yl)-3,4-dihydro-1H-isoquinolin-2-yl]-ethanone (Example 84) (0.1 g, 0.254 mmole) in 10 mL of dimethylformamide was treated with BOCA-aminobutyric acid (0.052 g, 0.256 mmole), triethylamine (0.14 mL, 1 mmole) and HATU (0.097 g, 1 mmole). Upon stirring for 16 h the reaction mixture was concentrated under reduced pressure, dissolved in dichloromethane and washed with potassium carbonate solution. The organic layer was dried over potassium carbonate and concentrated under reduced pressure to afford the desired material 1; LCMS (M+1) m/z 580.

### Example 89

### 4-Amino-N-(4-{2-[5-methoxy-8-(4-methyl-piperazin-1-yl)-3,4-dihydro-1H-isoquinolin-2-yl]-2-oxo-ethyl}-phenyl)-butyramide

A solution of [3-(4-{2-[5-Methoxy-8-(4-methyl-piperazin-1-yl)-3,4-dihydro-1H-isoquinolin-2-yl]-2-oxo-ethyl}-phenylcarbamoyl)-propyl]-carbamic acid tert-butyl ester (Example 89a) in 5 mL of trifluoroacetic acid was stirred for 30 min and concentrated under reduced pressure to afford the desired material 2 as trifluoroacetic acid salt (0.151 g); LCMS (M+1) m/z 480.5.

### Example 90

### 2-(4-Dibutylamino-phenyl)-1-[5-methoxy-8-(4-methyl-piperazin-1-yl)-3,4-dihydro-1H-isoquinolin-2-yl]-ethanone

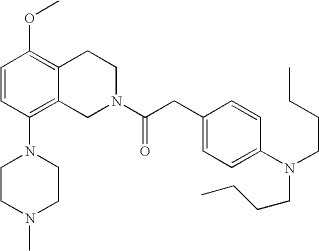

### Example 90a

### (4-Dibutylamino-phenyl)-acetic acid ethyl ester

A solution of 4-aminophenylacetic acid ethyl ester (0.896 g, 5 mmole) in 15 mL of methanol was treated with acetic acid (1.2 mL, 20 mmole) followed by butyraldehyde (0.8 g, 11 mmole) and sodiumcyanoborohydride (11.0 g). The reaction mixture was stirred for 16 h, concentrated under reduced pressure and diluted with dichloromethane. Upon washing with potassium carbonate solution the organic layer was dried over potassium carbonate, concentrated under reduced pressure and purified by chromatography over 40 g of silica gel to afford the desired material. (0.716 g); LCMS (M+1) m/z 292.

### Example 90b

### (4-Dibutylamino-phenyl)-acetic acid

A solution of (4-Dibutylamino-phenyl)-acetic acid ethyl ester (Example 90a) (0.71 g, 2.44 mmole) in 10 mL of tetrahydrofuran was treated with 6 mL of 1N lithium hydroxide solution. Upon stirring for 16 h the reaction mixture was acidified with concentrated hydrochloric acid and concentrated under reduced pressure to afford the desired material 2 (1.236 g); LCMS (M+1) m/z 264.

### Example 90

### 2-(4-Dibutylamino-phenyl)-1-[5-methoxy-8-(4-methyl-piperazin-1-yl)-3,4-dihydro-1H-isoquinolin-2-yl]-ethanone

A method similar to the one described for Example 87 was used except that 4-dibutyaminophenylacetic acid (Example 90b) was used instead of 4-isopropylphenylacetic acid to obtain the desired material. (0.09 g); LCMS (M+1) m-L/z 507.7.

### Example 91

### 2-(4-Butylamino-phenyl)-1-[5-methoxy-8-(4-methyl-piperazin-1-yl)-3,4-dihydro-1H-isoquinolin-2-yl]-ethanone

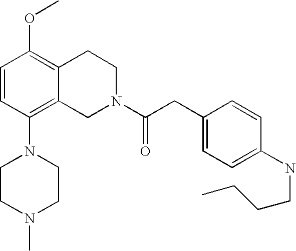

### Example 91a

### 4-butylaminphenylacetic acid ethyl ester

This compound was prepared by a method similar to the one described for 4-dibutylaminophenyacetic acid ethyl ester (Example 90a) as a by product; LCMS (M+1) m/z 236.

### Example 91b

### 4-butylaminophenylacetic acid

This compound was prepared by a method similar to the one described for 4-dibutylaminophenyacetic acid (Example 90b); LCMS (M+1) m/z 208.

### Example 91

### 2-(4-Butylamino-phenyl)-1-[5-methoxy-8-(4-methyl-piperazin-1-yl)-3,4-dihydro-1H isoquinolin-2-yl]-ethanone

This compound was prepared by a method similar to the one described for Example 90 except that 4-butylaminophenyacetic acid (Example 90b) was used instead of 4-dibutylaminphenylacetic acid; LCMS (M+1) m/z 451.6.

### Example 92

### 2-(4-Diphenethylamino-phenyl)-1-[5-methoxy-8-(4-methyl-piperazin-1-yl)-3,4-dihydro-1H-isoquinolin-2-yl]-ethanone

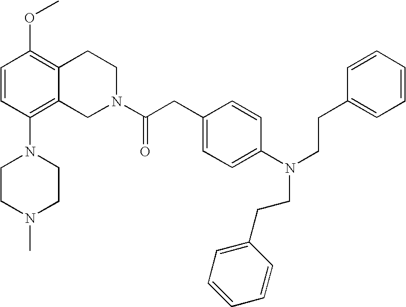

### Example 92a

### 4-di-phenethylaminphenylacetic acid ethyl ester

This compound was prepared by a method similar to the one described for 4-dibutylaminophenyacetic acid ethyl ester (Example 90a) except that phenyl acetaldehyde was used instead of butyraldehyde; LCMS (M+1) m/z 388.

### Example 92b

### 4-di-phenethylaminophenylacetic acid

This compound was prepared by a method similar to the one described for 4-dibutylaminophenyacetic acid (Example 90b) using Example 92a as starting material. LCMS (M+1) m/z 360.

### Example 92

### 2-(4-Diphenethylamino-phenyl)-1-[5-methoxy-8-(4-methyl-piperazin-1-yl)-3,4-dihydro-1H-isoquinolin-2-yl]-ethanone

A method similar to the one described for Example 87 was used except that 4-diphenethylamino-phenylacetic acid (Example 92b) was used instead of 4-isopropylphenylacetic acid to obtain the desired material (0.3 g); LCMS (M+1) m/z 603.6.

### Example 93

### 1-[5-Methoxy-8-(4-methyl-piperazin-1-yl)-3,4-dihydro-1H-isoquinolin-2-yl]-2-(4-phenethylamino-phenyl)-ethanone

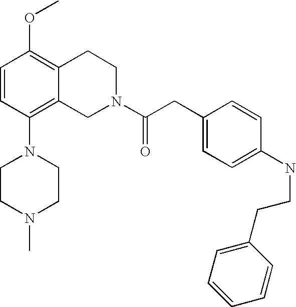

### Example 93a

### 4-phenyethylaminophenylacetic acid ethyl ester

This compound was prepared by a method similar to the one used for 4-butylaminophenylacetic acid ethyl ester (Example 91) except that phenyacetaldehyde was used instead of butyraldehyde. LCMS (M+1) m/z 284.

### Example 93b

### 4-phenylethlaminophenylacetic acid

This compound was prepared by a method similar to the one used for 4-butylaminophenylacetic acid (Example 91). LCMS (M+1) m/z 256.

### Example 93

### 1-[5-Methoxy-8-(4-methyl-piperazin-1-yl)-3,4-dihydro-1H-isoquinolin-2-yl]-2-(4-phenethylamino-phenyl)-ethanone

A method similar to the one described for Example 90 was used except that 4-phenethylaminophenylacetic acid (Example 93b) was used as the starting carboxylic acid. (0.17 g) LCMS (M+1) m/z 496.6.

### Example 94

### 2-{4-[Bis-(2-benzyloxy-ethyl)-amino]-phenyl}-1-[5-methoxy-8-(4-methyl-piperazin-1-yl)-3,4-dihydro-1H-isoquinolin-2-yl]-ethanone

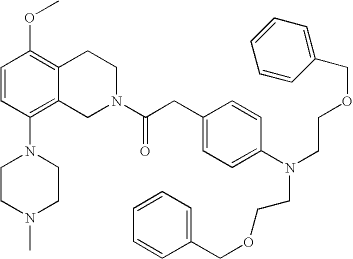

### Example 93a

### 4-(bis(2-phenoxyethyl))aminophenylacetic acid ethyl ester

This compound was prepared by a method similar to the one described for 4-dibutylaminophenyacetic acid ethyl ester (Example 90) except that benzyloxyacetaldehyde was used instead of butyraldehyde. LCMS (M+1) m/z 448.

### Example 93b

### 4-(bis(2-phenoxyethyl))aminophenylacetic acid

This compound was prepared by a method similar to the one described for 4-dibutylaminophenyacetic acid (Example 90). LCMS (M+1) m/z 420.

### Example 94

### 2-{4-[Bis-(2-benzyloxy-ethyl)-amino]-phenyl}-1-[5-methoxy-8-(4-methyl-piperazin-1-yl)-3,4-dihydro-1H-isoquinolin-2-yl]-ethanone

A method similar to the one described for Example 74 was used except that 4-(bis(2-phenoxyethyl))-aminophenylacetic acid (Example 93b) was used instead of 4-isopropylphenylacetic acid to obtain the desired material (0.193 g). LCMS (M+1) m/z 663.5.

### Example 95

### 2-[4-(2-Benzyloxy-ethylamino)-phenyl]-1-[5-methoxy-8-(4-methyl-piperazin-1-yl)-3,4-dihydro-1H-isoquinolin-2-yl]-ethanone

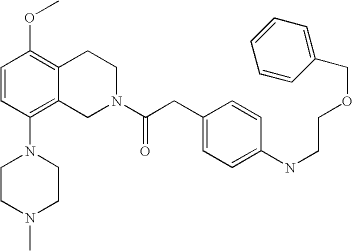

### Example 95a

### 4-(2-benzyloxyethylamino)phenylacetic acid ethyl ester

This compound was prepared by a method similar to the one used for 4-butylaminophenylacetic acid ethyl ester under Example 174 except that benzyloxyacetaldehyde was used instead of butyraldehyde. LCMS (M+1) m/z 309.

### Example 95b

### 4-(2-benzyloxyethylamino)phenylacetic acid

This compound was prepared by a method similar to the one used for 4-butylaminophenylacetic acid under Example 91. LCMS (M+1) m/z 286.

### Example 95

### 2-[4-(2-Benzyloxy-ethylamino)-phenyl]-1-[5-methoxy-8-(4-methyl-piperazin-1-yl)-3,4-dihydro-1H-isoquinolin-2-yl]-ethanone

A method similar to the one described for Example 90 was used except that 4-(2-benzyloxyethylamino)-phenylacetic acid (Example 95b) was used instead of 4-isopropylphenylacetic acid to obtain the desired material (0.197 g). LCMS (M+1) m/z 529.6.

### Example 96

### Biphenyl-4-yl-[5-methoxy-8-(4-methyl-piperazin-1-yl)-3,4-dihydro-1H-isoquinolin-2-yl]-methanone

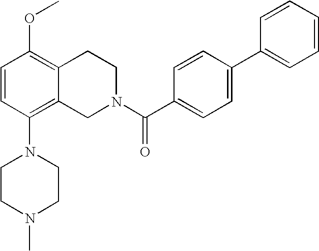

### Example 96

### Biphenyl-4-yl-[5-methoxy-8-(4-methyl-piperazin-1-yl)-3,4-dihydro-1H-isoquinolin-2-yl]-methanone

A method similar to the one described for Example 87 was used except that 4-phenylbenzoic acid was used instead of 4-isopropylphenylacetic acid to obtain the desired material (0.209 g); LCMS (M+1) m/z 442.6.

### Example 97

### 2-Biphenyl-4-yl-1-[5-methoxy-8-(4-methyl-piperazin-1-yl)-3,4-dihydro-1H-isoquinolin-2-yl)-ethanone

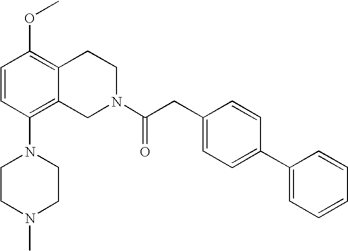

### Example 97

### 2-Biphenyl-4-yl-1-(5-methoxy-8-(4-methyl-piperazin-1-yl)-3,4-dihydro-1H-isoquinolin-2-yl]-ethanone

A method similar to the one described for Example 87 was used except that 4-phenylphenylacetic acid was used instead of 4-isopropylphenylacetic acid to obtain the desired material (0.2 g); LCMS (M+1) m/z 456.6.

### Example 98

### 1-[5-Methoxy-8-(4-methyl-piperazin-1-yl)-3,4-dihydro-1H-isoquinolin-2-yl]-2-(4-methoxy-phenyl)-ethanone

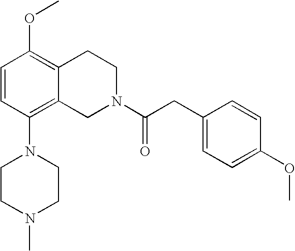

### Example 98

### 1-[5-Methoxy-8-(4-methyl-piperazin-1-yl)-3,4-dihydro-1H-isoquinolin-2-yl]-2-(4-methoxy-phenyl)-ethanone

A method similar to the one described for Example 87 was used except that 4-methoxyphenylacetic acid was used instead of 4-isopropylphenylacetic acid to obtain the desired material (0.2 g); LCMS (M+1) m/z 410.6.

### Example 99

### 2-Benzo[1,3]dioxol-5-yl-1-[5-methoxy-8-(4-methyl-piperazin-1-yl)-3,4-dihydro-1H-isoquinolin-2-yl]-ethanone

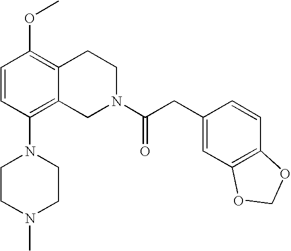

### Example 99

### 2-Benzo[1,3]dioxol-5-yl-1-[5-methoxy-8-(4-methyl-piperazin-1-yl)-3,4-dihydro-1H-isoquinolin-2-yl]-ethanone

A method similar to the one described for Example 87 was used except that 3,4-methylenedioxyphenylacetic acid was used instead of 4-isopropylphenylacetic acid to obtain the desired material (0.232 g); LCMS (M+1) m/z 424.5.

### Example 100

### 2-(3,4-Dimethoxy-phenyl)-1-[5-methoxy-8-(4-methyl-piperazin-1-yl)-3,4-dihydro-1H-isoquinolin-2-yl]-ethanone

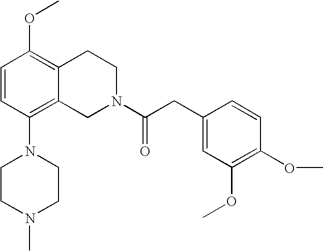

### Example 100

### 2-(3,4-Dimethoxy-phenyl)-1-[5-methoxy-8-(4-methyl-piperazin-1-yl)-3,4-dihydro-1H-isoquinolin-2-yl]-ethanone

A method similar to the one described for Example 87 was used except that 3,4-dimethoxyphenylacetic acid was used instead of 4-isopropylphenylacetic acid to obtain the desired material (0.270 g); LCMS (M+1) m/z 440.6.

### Example 101

### 2-(4-Fluoro-phenyl)-1-[5-methoxy-8-(4-methyl-piperazin-1-yl)-3,4-dihydro-1H-isoquinolin-2-yl]-ethanone

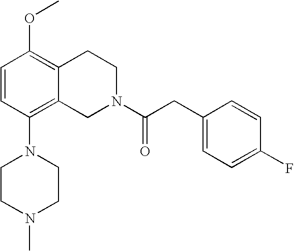

### Example 101

### 2-(4-Fluoro-phenyl)-1-[5-methoxy-8-(4-methyl-piperazin-1-yl)-3,4-dihydro-1H-isoquinolin-2-yl]-ethanone

A method similar to the one described for Example 87 was used except that 4-fluorophenylacetic acid was used instead of 4-isopropylphenylacetic acid to obtain the desired material (0.203 g); LCMS (M+1) m/z 398.5.

### Example 102

### 2-(4-Chloro-phenyl)-1-[5-methoxy-8-(4-methyl-piperazin-1-yl)-3,4-dihydro-1H-isoquinolin-2-yl]-ethanone

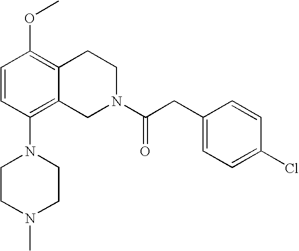

### Example 102

### 2-(4-Chloro-phenyl)-1-[5-methoxy-8-(4-methyl-piperazin-1-yl)-3,4-dihydro-1H-isoquinolin-2-yl]-ethanone

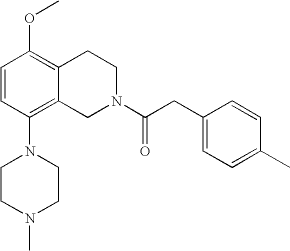

A method similar to the one described for Example 87 was used except that 4-chlorophenyl-acetic acid was used instead of 4-isopropylphenylacetic acid to obtain the desired material (0.180 g); LCMS (M+1) m/z 414.5.

### Example 103

### 2-(4-methyl-phenyl)-1-[5-methoxy-8-(4-methyl-piperazin-1-yl)-3,4-dihydro-1H-isoquinolin-2-yl]-ethanone

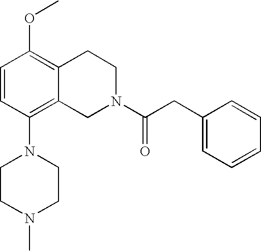

### Example 103

### 2-(4-methyl-phenyl)-1-[5-methoxy-8-(4-methyl-piperazin-1-yl)-3,4-dihydro-1H-isoquinolin-2-yl]-ethanone

A method similar to the one described for Example 87 was used except that 4-methylphenylacetic acid was used instead of 4-isopropylphenylacetic acid to obtain the desired material (0.234 g); LCMS (M+1) m/z 394.6.

### Example 104

### 2-Phenyl-1-[5-methoxy-8-(4-methyl-piperazin-1-yl)-3,4-dihydro-1H-isoquinolin-2-yl]-ethanone

### Example 104

### 2-Phenyl-1-[5-methoxy-8-(4-methyl-piperazin-1-yl)-3,4-dihydro-1H-isoquinolin-2-yl]-ethanone

A method similar to the one described for Example 87 was used except that phenylacetic acid was used instead of 4-isopropylphenylacetic acid to obtain the desired material (0.205 g);

LCMS (M+1) m/z 380.5.

### Example 105

### 1-[5-Methoxy-8-(4-methyl-piperazin-1-yl)-3,4-dihydro-1H-isoquinolin-2-yl]-2-(4-methylsulfanyl-phenyl)-ethanone

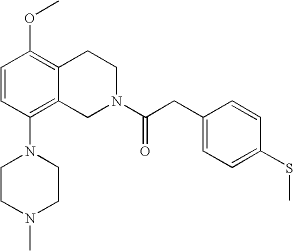

### Example 105

### 1-[5-Methoxy-8-(4-methyl-piperazin-1-yl)-3,4-dihydro-1H-isoquinolin-2-yl]-2-(4-methylsulfanyl-phenyl)-ethanone

A method similar to the one described for Example 87 was used except that 4-thiomethylphenylacetic acid was used instead of 4-isopropylphenylacetic acid to obtain the desired material (0.233 g); LCMS (M+1) m/z 426.6.

### Example 106

### 2-(4-Methanesulfinyl-phenyl)-1-[5-methoxy-8-(4-methyl-piperazin-1-yl)-3,4-dihydro-1H-isoquinolin-2-yl]-ethanone

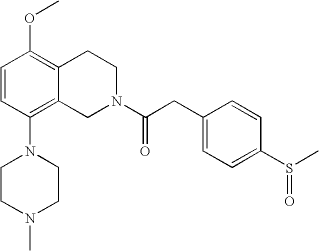

### Example 106a

### 4-sulfinylmethylphenylacetic acid

A solution of 4-(methylthio)phenylacetic acid (0.364 g, 2 mmole) in 15 mL of tetrahydrofuran was treated with a solution of sodium periodate (2.14 g, 10 mmole) in 20 mL of water. Upon stirring for 16 h the reaction mixture was diluted with dichloromethane and extracted with water. The organic layer was dried over magnesium sulfate and concentrated under reduced pressure to afford the desired product; LCMS (M+1) m/z 199.

### Example 106

### 2-(4-Methanesulfinyl-phenyl)-1-[5-methoxy-8-(4-methyl-piperazin-1-yl)-3,4-dihydro-1H-isoquinolin-2-yl]-ethanone

A method similar to the one described for Example 87 was used except that 4-sulfinylmethylphenylacetic acid (Example 106a) was used instead of 4-isopropylphenylacetic acid to obtain the desired material (0.280 g); LCMS (M+1) m/z 442.5.

### Example 107

### N-(4-{2-[5-Methoxy-8-(4-methyl-piperazin-1-yl)-3,4-dihydro-1H-isoquinolin-2-yl]-2-oxo-ethyl}-phenyl)-methanesulfonamide

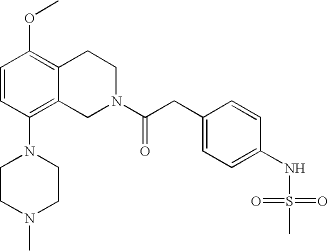

### Example 107a

### 4-sulfonamidophenylacetic acid diethyl ester

A solution of 4-aminophenylacetic acid ethyl ester (0.358 g, 2 mmole) in 20 mL of dichloromethane at 0 C was treated with triethylamine (0.56 ml, 4 mmole) and methanesulfonylchloride (0.318 g, 2.2 mmole). Upon stirring for 2 h the reaction mixture was diluted with dichloromethane, washed with 5% hydrochloric acid and sodium bicarbonate, dried over magnesium sulfate and concentrated under reduced pressure to afford the desired product. (0.546 g); LCMS (M-42) m/z 299.4.

### Example 107b

### 4-sulfonamidophenylacetic acid

A solution of 4-sulfonamidophenylacetic acid diethyl ester (Example 107a) (0.546 g) in 40 mL of methanol was treated with 3 mL of 1N lithium hydroxide and refluxed for 16 h. At the end of this period the reaction mixture was concentrated under reduced pressure, dissolved in 20 mL of water and extracted with ether. The aqueous layer was acidified with 5% hydrochloric acid, extracted three times with dichloromethane, the organic layers were dried over magnesium sulfate and concentrated under reduced pressure to afford the desired material (0.236 g) LCMS (M+1) m/z 230.

### Example 107

### N-(4-{2-[5-Methoxy-8-(4-methyl-piperazin-1-yl)-3,4-dihydro-1H-isoquinolin-2-yl]-2-oxo-ethyl}-phenyl)-methanesulfonamide

A method similar to the one described for Example 87 was used except that 4-sulfonamidophenylacetic acid (Example 107b) was used instead of 4-isopropylphenylacetic acid to obtain the desired material (0.303 g); LCMS (M+1) m/z 473.48.

### Example 108

### 2-[4-(2-Methoxy-benzylamino)-phenyl]-1-[5-methoxy-8-(4-methyl-piperazin-1-yl)-3,4-dihydro-1H-isoquinolin-2-yl]-ethanone

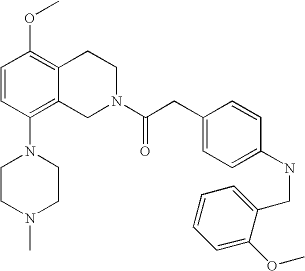

### Example 108a

### 4(2-methoxybenzylamino)phenylacetic acid ethyl ester

A solution of p-aminophenylacetic acid ethyl ester (1.79 g, 10 mmole) in 10 mL of methanol was treated with o-anisaldehyde (1.36 g, 10 mmole) and 6 mL of acetic acid followed by sodiumcyanoborohydride (1.0 g). Upon stirring for 16 h the reaction mixture was diluted with dichloromethane, washed with potassium carbonate solution, dried over potassium carbonate and concentrated under reduced pressure to obtain the crude product. Purification of 0.25 g by fcc afforded the desired product (0.14 g).

### Example 108b

### 4(2-methoxybenzylamino)phenylacetic acid

A solution of 4(2-methoxybenzylamino)phenylacetic acid ethyl ester (Example 108a) in 10 mL of methanol was treated with 2 mL of 1N lithium hydroxide and stirred for 16 h. At the end of this period the reaction mixture was acidified with concentrated hydrochloric acid and concentrated under reduced pressure to afford the desired 4(2-methoxybenzylamino)phenyl-acetic acid.

### Example 108

### 2-[4-(2-Methoxy-benzylamino)-phenyl]-1-[5-methoxy-8-(4-methyl-piperazin-1-yl)-3,4-dihydro-1H-isoquinolin-2-yl]-ethanone

A method similar to the one described for Example 87 was used except that 4(2-methoxybenzylamino)phenylacetic acid (Example 108b) was used instead of 4-isopropylphenylacetic acid to obtain the desired material (0.201 g); LCMS (M+1) m/z 515.5.

### Example 109

### 2-(4-Benzylamino-phenyl)-1-[5-methoxy-8-(4-methyl-piperazin-1-yl)-3,4-dihydro-1H-isoquinolin-2-yl]-ethanone

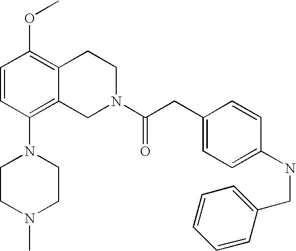

### Example 109

### 2-(4-Benzylamino-phenyl)-1-[5-methoxy-8-(4-methyl-piperazin-1-yl)-3,4-dihydro-1H-isoquinolin-2-yl]-ethanone

A method similar to the one described for Example 87 was used except that 4-benzylaminophenylacetic acid was used instead of 4-isopropylphenylacetic acid to obtain the desired material (0.201 g); LCMS (M+1) m/z 485.5.

Methods similar to those described under Example 108 were used except that benzaldehyde was used instead of o-anisaldehyde to obtain the desired 4-benzylaminophenylacetic acid.

### Example 110

### 2-[4-(3-Methoxy-benzylamino)-phenyl]-1-[5-methoxy-8-(4-methyl-piperazin-1-yl)-3,4-dihydro-1H-isoquinolin-2-yl)-ethanone

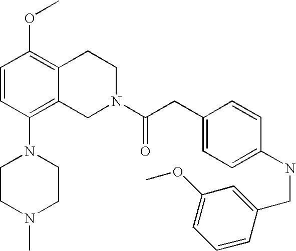

### Example 110

### 2-[4-(3-Methoxy-benzylamino)-phenyl]-1-[5-methoxy-8-(4-methyl-piperazin-1-yl)-3,4-dihydro-1H-isoquinolin-2-yl]-ethanone

A method similar to the one described for Example 87 was used except that 4(3-methoxybenzylamino)phenylacetic acid was used instead of 4-isopropylphenylacetic acid to obtain the desired material (0.201 g); LCMS (M+1) m/z 485.5.

Methods similar to those described under Example 108 were used except that m-anisaldehyde was used instead of o-anisaldehyde to obtain the desired 4(3-methoxybezylamino)phenyl-acetic acid.

### Example 111

### 2-[4-(4-Methoxy-benzylamino)-phenyl]-1-[5-methoxy-8-(4-methyl-piperazin-1-yl)-3,4-dihydro-1H-isoquinolin-2-yl]-ethanone

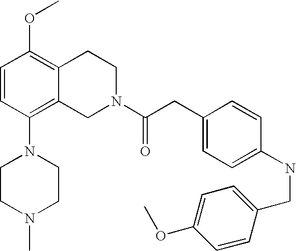

### Example 111

### 2-[4-(4-Methoxy-benzylamino)-phenyl]-1-[5-methoxy-8-(4-methyl-piperazin-1-yl)-3,4-dihydro-1H-isoquinolin-2-yl]-ethanone

A method similar to the one described for Example 87 was used except that 4(4-methoxybenzylamino)-phenylacetic acid was used instead of 4-isopropylphenylacetic acid to obtain the desired material (0.153 g); LCMS (M+1) m/z 485.5. Methods similar to those described under Example 108 were used except that p-anisaldehyde was used instead of o-anisaldehyde to obtain the desired 4(4-methoxybezylamino)phenyl-acetic acid.

### Example 112

### 2-(4-Isopropyl-phenyl)-1-[5-methoxy-8-(4-methyl-piperazine-1-carbonyl)-3,4-dihydro-1H-isoquinolin-2-yl]-ethanone

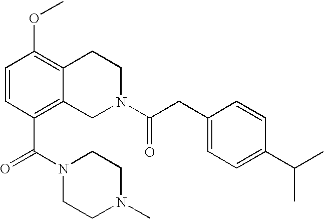

### Example 112a

### 8-Bromo-5-methoxy-1,2,3,4-tetrahydro-isoquinoline

A solution of 8-Bromo-5-methoxy-isoquinoline (2.37 g, 10 mmole) in 50 mL of methanol was treated with sodiumcyanoborohydride (2.19 g, 35 mmole) followed by boron trifluoride etherate (4.4 mL, 35 mmole) and the reaction mixture was refluxed for 2 h. At the end of this period the reaction mixture was cooled to the room temperature, treated with the same amount of sodium cyanoborohydride and boron trifluoride etherate as before, refluxed for 2 h and poured in potassium carbonate solution. Upon extracting with dichloromethane the organic layer was filtered through diatomaceous earth, dried over potassium carbonate and concentrated under reduced pressure to afford the desired product (2.34 g); LCMS (M+1) m/z 242.

### Example 112b

### 1-(8-Bromo-5-methoxy-3,4-dihydro-1H-isoquinolin-2-yl)-2-(4-isopropyl-phenyl)-ethanone

A solution of 8-Bromo-5-methoxy-1,2,3,4-tetrahydro-isoquinoline (Example 112b) (2.34 g, 9.7 mmole) in 100 ml of dichloromethane was treated with 4-isopropylphenylacetic acid (2.14 g, 12 mmole), triethylamine (2.8 mmole, 2.8 mL) and HATU (3.8 g, 10 mmole). Upon stirring for 16 h the reaction mixture was diluted with dichloromethane, washed with 5% hydrochloric acid and sodium bicarbonate and dried over magnesium sulfate. Concentration under reduced pressure and purification on silica gel afforded the desired product (3.6 g);

LCMS (M+1) m/z 403.

### Example 112c

### 2-[2-(4-Isopropyl-phenyl)-acetyl]-5-methoxy-1,2,3,4-tetrahydro-isoquinoline-8-carboxylic acid methyl ester

A solution of 1-(8-Bromo-5-methoxy-3,4-dihydro-1H-isoquinolin-2-yl)-2-(4-isopropyl-phenyl)-ethanone (Example 112c) (0.517 g, 1.28 mmole) in 10 mL of DMSO and 10 mL of methanol was treated with triethylamine (0.39 mL, 2.8 mmole), palladium acetate (14 mg, 0.064 mmole), and dppp (26 mg, 0.064 mmole). Upon passing Co for 15 min while heating to 70 C the reaction mixture was heated to 70 C for 16 h under an atmosphere of CO. At the end of this period the reaction mixture diluted with 1:1 hexane:ethylacetate, washed four times with water and dried over magnesium sulfate. Upon concentration under reduced pressure the product was purified by fcc to afford the desired product (69 mg); LCMS (M+1) m/z 382.

### Example 112d

### 2-[2-(4-Isopropyl-phenyl)-acetyl]-5-methoxy-1,2,3,4-tetrahydro-isoquinoline-8-carboxylic acid

A solution of 2-[2-(4-Isopropyl-phenyl)-acetyl]-5-methoxy-1,2,3,4-tetrahydro-isoquinoline-8-carboxylic acid methyl ester (Example 112c) (0.242 g, 0.64 mmole) in 10 mL of methanol was treated with 1 mlL of 1N lithium hydroxide, 10 mL of water and refluxed for 16 h. At the end of this period the reaction mixture was cooled to the room temperature, concentrated under reduced pressure, diluted with 20 mL of water and extracted with ether twice. The aqueous layer was acidified with 5% hydrochloric acid and extracted with ethyl acetate twice. The combined organic layers were dried over magnesium sulfate, concentrated under reduced pressure to obtain the desired product (0.176 g); LCMS (M+1) m/z 382.

### Example 112

### 2-(4-Isopropyl-phenyl)-1-[5-methoxy-8-(4-methyl-piperazine-1-carbonyl)-3,4-dihydro-1H-isoquinolin-2-yl]-ethanone

A suspension of 2-[2-(4-Isopropyl-phenyl)-acetyl]-5-methoxy-1,2,3,4-tetrahydro-isoquinoline-8-carboxylic acid (Example 112d) (0.176 g, 0.48 mmole) in 30 mL of dichloromethane was treated with N-methylpiperizine (0.11 mL, 1 mmole), triethylamine (0.14 mL, 1 mmole) and HATU (0.19 g, 0.5 mmole). Upon stirring for 16 h the reaction mixture was diluted with dichloromethane and washed with a solution of potassium carbonate, dried over potassium carbonate and concentrated under reduced pressure to afford the crude product. This material was purified by fcc to obtain the desired material (0.135 g);

LCMS (M+1) m/z 450.5.

### Example 113

### 5-Methoxy-8-(4-methyl-piperazin-1-yl)-3,4-dihydro-1H-isoquinoline-2-carboxylic acid (4-isopropyl-phenyl)-amide

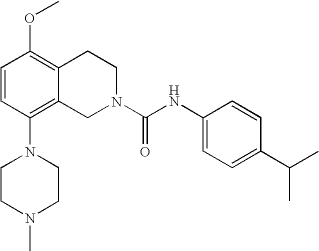

### Example 113

### 5-Methoxy-8-(4-methyl-piperazin-1-yl)-3,4-dihydro-1H-isoquinoline-2-carboxylic acid (4-isopropyl-phenyl)-amide

A solution of 4-isopropylaniline (0.135 g, 1 mmole) in 2 mL of dichloromethane was treated with carbonyldiimidazole (0.162 g, 1 mmole) and stirred for 16 h. At the end of this period 5-Methoxy-8-(4-methyl-piperazin-1-yl)-1,2,3,4-tetrahydro-isoquinoline (0.262 g, 1 mmole) was added to the reaction mixture, stirred for 2 h and the product was purified by fcc to afford the desired product (45 mg); LCMS (M+1) m/z 423.

### Example 114

### 5-Methoxy-8-(4-methyl-piperazin-1-yl)-3,4-dihydro-1H-isoquinoline-2-carboxylic acid (4-cyclohexyl-phenyl)-amide

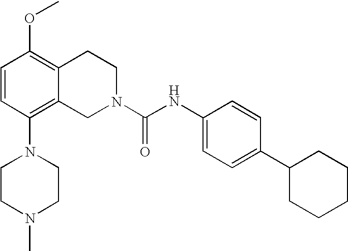

### Example 114

### 5-Methoxy-8-(4-methyl-piperazin-1-yl)-3,4-dihydro-1H-isoquinoline-2-carboxylic acid (4-cyclohexyl-phenyl)-amide

A method similar to the one used for Example 113 was used except that 4-cyclohexylaniline (0.175 g, 1 mmole) was used instead of 4-isopropylaniline to obtain the desired material (0.168 g); LCMS (M+1) m/z 463.

### Example 115

### 5-Methoxy-8-(4-methyl-piperazin-1-yl)-3,4-dihydro-1H-isoquinoline-2-carboxylic acid [4-(5-methoxy-pyrimidin-2-ylsulfamoyl)-phenyl]-amide

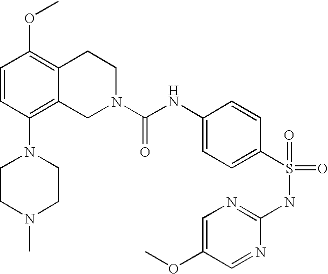

### Example 115

### 5-Methoxy-8-(4-methyl-piperazin-1-yl)-3,4-dihydro-1H-isoquinoline-2-carboxylic acid (4-(5-methoxy-pyrimidin-2-ylsulfamoyl)-phenyl]-amide

A method similar to the one used for Example 113 was used except that sulfameter (0.280 g, 1 mmole) was used instead of 4-isopropylaniline and purification was done using prepHPLC to obtain the desired material (0.230 g); LCMS (M+1) m/z 463.

### Example 116

### (4-{[5-Methoxy-8-(4-methyl-piperazin-1-yl)-3,4-dihydro-1H-isoquinoline-2-carbonyl]-amino}-benzyl)-phosphonic acid diethyl ester

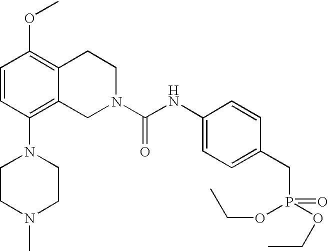

### Example 116

### (4-{[5-Methoxy-8-(4-methyl-piperazin-1-yl)-3,4-dihydro-1H-isoquinoline-2-carbonyl]-amino}-benzyl)-phosphonic acid diethyl ester

A method similar to the one used for Example 113 was used except that diethyl-4-amino-benzylphosphonate (0.243 g, 1 mmole) was used instead of 4-isopropylaniline and purification was done using prepHPLC to obtain the desired material (0.300 g); LCMS (M+1) m/z 532.

### Example 117

### 5-Methoxy-8-(4-methyl-piperazin-1-yl)-3,4-dihydro-1H-isoquinoline-2-carboxylic acid [4-(3,4-dimethyl-isoxazol-5-ylsulfamoyl)-phenyl]-amide

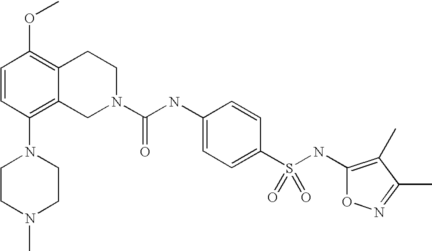

### Example 117

### 5-Methoxy-8-(4-methyl-piperazin-1-yl)-3,4-dihydro-1H-isoquinoline-2-carboxylic acid [4-(3,4-dimethyl-isoxazol-5-ylsulfamoyl)-phenyl]-amide

A method similar to the one used for Example 113 was used except that sulfisooxazole was used instead of 4-isopropylaniline and the reaction was done on twice the scale to obtain the desired material (95 mg); LCMS (M+1) m/z 554.4

### Example 118

### 5-Methoxy-8-(4-methyl-piperazin-1-yl)-3,4-dihydro-1H-isoquinoline-2-carboxylic acid [4-(6-methyl-benzothiazol-2-yl)-phenyl]-amide

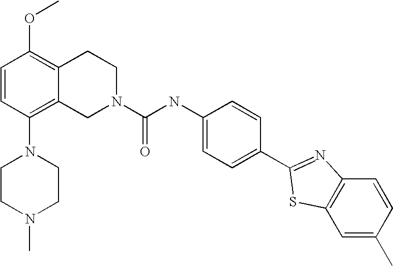

### Example 118

### 5-Methoxy-8-(4-methyl-piperazin-1-yl)-3,4-dihydro-1H-isoquinoline-2-carboxylic acid [4-(6-methyl-benzothiazol-2-yl)-phenyl]-amide

A method similar to the one used for Example 113 was used except that 2(4-aminophenyl)-6-methylbenzothiazole was used instead of 4-isopropylaniline and the reaction was done on twice the scale to obtain the desired material (230 mg); LCMS (M+1) m/z 528.4

### Example 119

### 5-Methoxy-8-(4-methyl-piperazin-1-yl)-3,4-dihydro-1H-isoquinoline-2-carboxylic acid (4-tert-butyl-phenyl)-amide

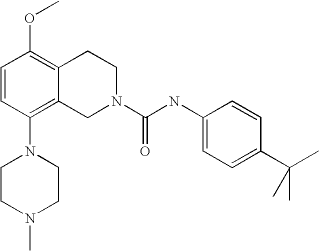

### Example 119

### 5-Methoxy-8-(4-methyl-piperazin-1-yl)-3,4-dihydro-1H-isoquinoline-2-carboxylic acid (4-tert-butyl-phenyl)-amide

A method similar to the one used for Example 113 was used except that 4-tert-butylaniline was used instead of 4-isopropylaniline and the reaction was done on twice the scale to obtain the desired material (252 mg); LCMS (M+1) m/z 437.5.

### Example 120

### 5-Methoxy-8-(4-methyl-piperazin-1-yl)-3,4-dihydro-1H-isoquinoline-2-carboxylic acid (4-sulfamoyl-phenyl)-amide

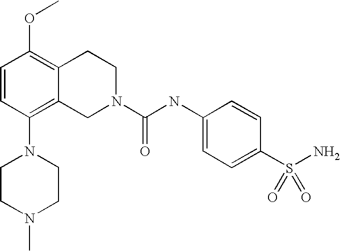

### Example 120

### 5-Methoxy-8-(4-methyl-piperazin-1-yl)-3,4-dihydro-1H-isoquinoline-2-carboxylic acid (4-sulfamoyl-phenyl)-amide

A method similar to the one used for Example 113 was used except that sulfanilamide was used instead of 4-isopropylaniline and the reaction was done on twice the scale. Purification was done using prepHPLC to obtain the desired material (10 mg); LCMS (M+1) m/z 460.

### Example 121

### 5-Methoxy-8-(4-methyl-piperazin-1-yl)-3,4-dihydro-1H-isoquinoline-2-carboxylic acid [4-(2-phenyl-2H-pyrazol-3-ylsulfamoyl)-phenyl]-amide

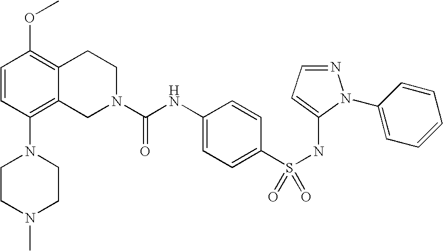

### Example 121

### 5-Methoxy-8-(4-methyl-piperazin-1-yl)-3,4-dihydro-1H-isoquinoline-2-carboxylic acid [4-(2-phenyl-2H-pyrazol-3-ylsulfamoyl)-phenyl]-amide

A method similar to the one used for Example 113 was used except that sulfaphenazole was used instead of 4-isopropylaniline and the reaction was done on twice the scale. Purification was done using fcc to obtain the desired material (265 mg); LCMS (M+1) m/z 602.35.

### Example 122

### 5-Methoxy-8-(4-methyl-piperazin-1-yl)-3,4-dihydro-1H-isoquinoline-2-carboxylic acid [4-(pyrrolidine-1-sulfonyl)-phenyl]-amide

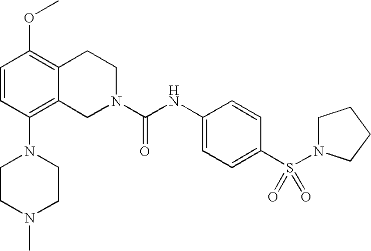

### Example 122

### 5-Methoxy-8-(4-methyl-piperazin-1-yl)-3,4-dihydro-1H-isoquinoline-2-carboxylic acid [4-(pyrrolidine-1-sulfonyl)-phenyl]-amide

A method similar to the one used for Example 113 was used except that N(4-aminophenylsulfonyl)-pyrrolidine was used instead of 4-isopropylaniline and the reaction was done on twice the scale. Purification was done using fcc and preparativeHPLC to obtain the desired material (30 mg); LCMS (M+1) m/z 514.49.

### Example 123

### 5-Methoxy-8-(4-methyl-piperazin-1-yl)-3,4-dihydro-1H-isoquinoline-2-carboxylic acid [4-(5-methyl-[1,3,4]thiadiazol-2-ylsulfamoyl)-phenyl]-amide

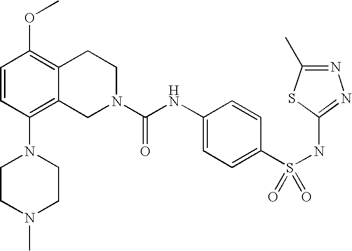

### Example 123

### 5-Methoxy-8-(4-methyl-piperazin-1-yl)-3,4-dihydro-1H-isoquinoline-2-carboxylic acid [4-(5-methyl-[1,3,4]thiadiazol-2-ylsulfamoyl)-phenyl]-amide

A method similar to the one used for Example 113 was used except that sulfmethiozole was used instead of 4-isopropylaniline and the reaction was done on twice the scale. Purification was done using preparative HPLC to obtain the desired material (43 mg); LCMS (M+1) m/z 558.38.

### Example 124

### 5-Methoxy-8-(4-methyl-piperazin-1-yl)-3,4-dihydro-1H-isoquinoline-2-carboxylic acid [4-(4,5-dimethyl-oxazol-2-ylsulfamoyl)-phenyl]-amide

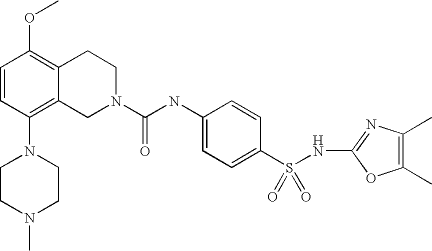

### Example 124

### 5-Methoxy-8-(4-methyl-piperazin-1-yl)-3,4-dihydro-1H-isoquinoline-2-carboxylic acid [4-(4,5-dimethyl-oxazol-2-ylsulfamoyl)-phenyl]-amide

A method similar to the one used for Example 113 was used except that sulfamoxole was used instead of 4-isopropylaniline and the reaction was done on twice the scale. Purification was done using preparative HPLC to obtain the desired material (27 mg); LCMS (M+1) m/z 555.26.

### Example 125

### 5-Methoxy-8-(4-methyl-piperazin-1-yl)-3,4-dihydro-1H-isoquinoline-2-carboxylic acid [4-(2-phenyl-2H-pyrazol-3-ylsulfamoyl)-phenyl]-amide

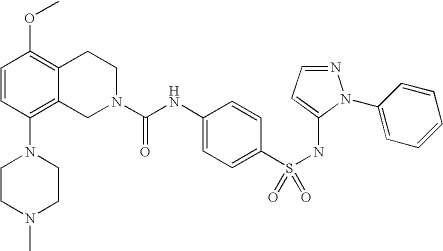

### Example 125

### 5-Methoxy-8-(4-methyl-piperazin-1-yl)-3,4-dihydro-1H-isoquinoline-2-carboxylic acid [4-(2-phenyl-2H-pyrazol-3-ylsulfamoyl)-phenyl]-amide

A method similar to the one used for Example 113 was used except that sulfaphenazole was used instead of 4-isopropylaniline and the reaction was done on twice the scale. Purification was done by fcc to obtain the desired material (265 mg); LCMS (M+1) m/z 602.35.

### Example 126

### 5-Methoxy-8-(4-methyl-piperazin 1-yl)-3,4-dihydro-1H-isoquinoline-2-carboxylic acid [4-(4-methyl-pyrimidin-2-ylsulfamoyl)-phenyl]-amide

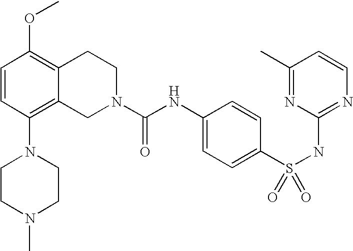

### Example 126

### 5-Methoxy-8-(4-methyl-piperazin-1-yl)-3,4-dihydro-1H-isoquinoline-2-carboxylic acid [4-(4-methyl-pyrimidin-2-ylsulfamoyl)-phenyl]-amide

A suspension of sulfamerazine (0.369 g, 1.4 mmole) in 10 mL of dichloromethane at 0 C was treated with triphosgene (0.123 g, 0.466 mmole) followed by triethylamine (0.42 mL, 3 mmole). The reaction mixture was allowed to reach room temperature while stirring for 1 h and treated with 5-Methoxy-8-(4-methyl-piperazin-1-yl)-1,2,3,4-tetrahydro-isoquinoline (Example 1d) (0.367 g, 1.4 mmole) followed by triethylamine (0.42 mL, 3 mmole). After stirring for 1 h the reaction mixture was diluted with dichloromethane and washed with sodium carbonate solution. The organic layer was dried over potassium carbonate and concentrated under reduced pressure to afford the crude product which was purified by fcc to obtain the desired material (0.223). LCMS (M+1) m/z 552.41.

### Example 127

### 5-Methoxy-8-(4-methyl-piperazin-1-yl)-3,4-dihydro-1H-isoquinoline-2-carboxylic acid [4-(2,6-dimethyl-pyrimidin-4-ylsulfamoyl)-phenyl]-amide

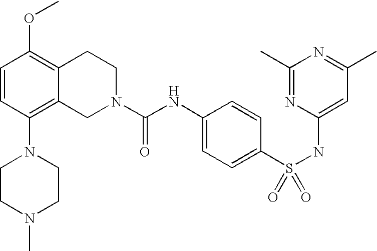

### Example 127

### 5-Methoxy-8-(4-methyl-piperazin-1-yl)-3,4-dihydro-1H-isoquinoline-2-carboxylic acid [4-(2,6-dimethyl-pyrimidin-4-ylsulfamoyl)-phenyl]-amide

This compound was prepared using a method similar to the one described for Example 125 except that 1 mmole of sulfisomidine was used in place of sulfamerazine and other reagents were adjusted accordingly. The product was purified by fcc to obtain the desired compound (38 mg); LCMS (M+1) m/z 566.

### Example 128

### 5-Methoxy-8-(4-methyl-piperazin-1-yl)-3,4-dihydro-1H-isoquinoline-2-carboxylic acid [4-(pyrimidin-2-ylsulfamoyl)-phenyl]-amide

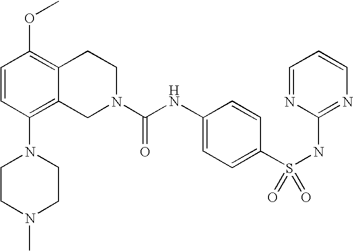

### Example 128

### 5-Methoxy-8-(4-methyl-piperazin-1-yl)-3,4-dihydro-1H-isoquinoline-2-carboxylic acid [4-(pyrimidin-2-ylsulfamoyl)-phenyl]-amide

This compound was prepared using a method similar to the one described for Example 125 except that 1 mmole of sulfadiazine was used in place of sulfamerazine and other reagents were adjusted accordingly. The product was purified by fcc to obtain the desired compound (102 mg); LCMS (M+1) m/z 538.

### Example 129

### 5-Methoxy-8-(4-methyl-piperazin-1-yl)-3,4-dihydro-1H-isoquinoline-2-carboxylic acid [4-(2,6-dimethoxy-pyrimidin-4-ylsulfamoyl)-phenyl]-amide

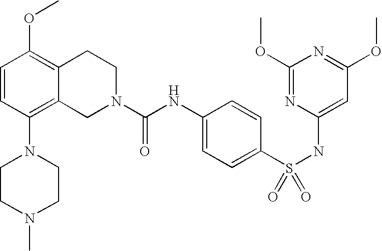

### Example 129

### 5-Methoxy-8-(4-methyl-piperazin-1-yl)-3,4-dihydro-1H-isoquinoline-2-carboxylic acid [4-(2,6-dimethoxy-pyrimidin-4-ylsulfamoyl)-phenyl]-amide

This compound was prepared using a method similar to the one described for Example 125 except that 1 mmole of sulfadimethoxin was used in place of sulfamerazine and other reagents were adjusted accordingly. The product was purified by fcc to obtain the desired compound. (315 mg); LCMS (M+1) m/z 597.

### Example 130

### 5-Methoxy-8-(4-methyl-piperazin-1-yl)-3,4-dihydro-1H-isoquinoline-2-carboxylic acid [4-(6-methoxy-pyridazin-3-ylsulfamoyl)-phenyl]-amide

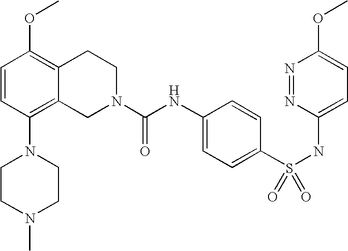

### Example 130

### 5-Methoxy-8-(4-methyl-piperazin-1-yl)-3,4-dihydro-1H-isoquinoline-2-carboxylic acid [4-(6-methoxy-pyridazin-3-ylsulfamoyl)-phenyl]-amide

This compound was prepared using a method similar to the one described for Example 125 except that 1 mmole of sulfamethoxypyridazine was used in place of sulfamerazine and other reagents were adjusted accordingly. The product was purified by fcc to obtain the desired compound. (286 mg); LCMS (M+1) m/z 567.

### Example 131

### 5-Methoxy-8-(4-methyl-piperazin-1-yl)-3,4-dihydro-1H-isoquinoline-2-carboxylic acid [4-(4,6-dimethyl-pyrimidin-2-ylsulfamoyl)-phenyl]-amide

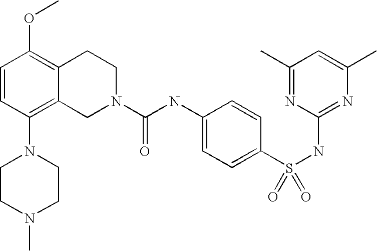

### Example 131

### 5-Methoxy-8-(4-methyl-piperazin-1-yl)-3,4-dihydro-1H-isoquinoline-2-carboxylic acid [4-(4,6-dimethyl-pyrimidin-2-ylsulfamoyl)-phenyl]-amide

This compound was prepared using a method similar to the one described for Example 125 except that 1 mmole of sufanethazine was used in place of sulfamerazine and other reagents were adjusted accordingly. The product was purified by fcc to obtain the desired compound. (140 mg); LCMS (M+1) m/z 565

### Example 132

### 5-Methoxy-8-(4-methyl-piperazin-1-yl)-3,4-dihydro-1H-isoquinoline-2-carboxylic acid [4-(6-methoxy-pyrimidin-4-ylsulfamoyl)-phenyl]-amide

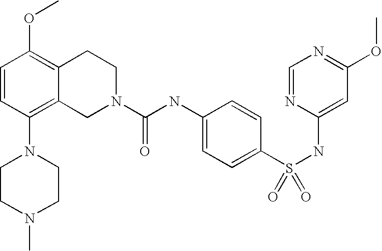

### Example 132

### 5-Methoxy-8-(4-methyl-piperazin-1-yl)-3,4-dihydro-1H-isoquinoline-2-carboxylic acid [4-(6-methoxy-pyrimidin-4-ylsulfamoyl)-phenyl]-amide

This compound was prepared using a method similar to the one described for Example 125 except that 1 mmole of sulfamonomethoxine was used in place of sulfamerazine and other reagents were adjusted accordingly. The product was purified by fcc to obtain the desired compound. (140 mg); LCMS (M+1) m/z 565.

### Example 133

### 5-Methoxy-8-(4-methyl-piperazin-1-yl)-3,4-dihydro-1H-isoquinoline-2-carboxylic acid [4-(pyridin-2-ylsulfamoyl)-phenyl]-amide

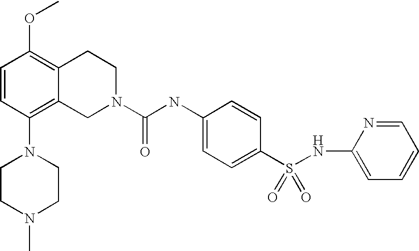

### Example 133

### 5-Methoxy-8-(4-methyl-piperazin-1-yl)-3,4-dihydro-1H-isoquinoline-2-carboxylic acid [4-(pyridin-2-ylsulfamoyl)-phenyl]-amide

This compound was prepared using a method similar to the one described for Example 125 except that 1 mmole of sulfapyridine was used in place of sulfamerazine and other reagents were adjusted accordingly. The product was purified by fcc to obtain the desired compound (140 mg); LCMS (M+1) m/z 565.

### Example 134

### 4-{2-[5-Methoxy-8-(4-methyl-piperazin-1-yl)-3,4-dihydro-1H-isoquinolin-2-yl]-2-oxo-ethyl}-benzoic acid methyl ester

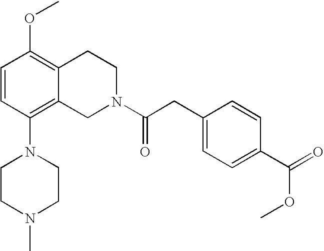

### Example 134

### 4-{2′-[5-Methoxy-8-(4-methyl-piperazin-1-yl)-3,4-dihydro-1H-isoquinolin-2-yl]-2-oxo-ethyl}-benzoic acid methyl ester

A solution of 2-(4-Bromo-phenyl)-1-[5-methoxy-8-(4-methyl-piperazin-1-yl)-3,4-dihydro-1H-isoquinolin-2-yl]-ethanone (Example 74a) (10 mmole) in 40 mL of 1:1 mixture of methanol and DMSO was treated with triethylamine (3.08 mL, 22 mmole), palladium acetate (0.224 g, 1.0 mmole), dppp (0.412 g, 1 mmole) and Co was for 15 min passed while heating to 70. The resulting reaction mixture was heated to 70 C for 16 h under an atmosphere of CO. At the end of this period the reaction mixture was concentrated under reduced pressure and chromatographer over silica gel to afford the desired product (3.28 g); LCMS (M+1) m/z 438.

### Example 135

### 4-{2-[5-Methoxy-8-(4-methyl-piperazin-1-yl)-3,4-dihydro-1H-isoquinolin-2-yl]-2-oxo-ethyl}-N-methyl-benzamide

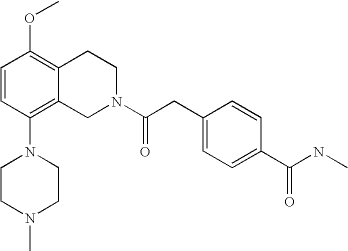

### Example 135a

### 4-{2-[5-Methoxy-8-(4-methyl-piperazin-1-yl)-3,4-dihydro-1H-isoquinolin-2-yl]-2-oxo-ethyl}-benzoic acid

A solution of 4-{2-[5-Methoxy-8-(4-methyl-piperazin-1-yl)-3,4-dihydro-1H-isoquinolin-2-yl]-2-oxo-ethyl}-benzoic acid methyl ester 2 (3.0 g, 6.86 mmole) in 50 mL of methanol was treated with 20 mL of 1N sodium hydroxide and refluxed for 16 h. At the end of this period the reaction mixture was treated with 2 mL of concentrated hydrochloric acid and concentrated under reduce pressure to afford the desired product (6.594 g) which was stirred in 50 mL of dichloromethane to afford a solid (2.11 g); LCMS (M+1) m/z 424.

### Example 135

### 4-{2-(5-Methoxy-8-(4-methyl-piperazin-1-yl)-3,4-dihydro-1H-isoquinolin-2-yl]-2-oxo-ethyl}-N-methyl-benzamide

A method similar to the one described for Example 85 was used except that 4-{2-[5-Methoxy-8-(4-methyl-piperazin-1-yl)-3,4-dihydro-1H-isoquinolin-2-yl]-2-oxo-ethyl}-benzoic acid (Example 135a) was used instead of 4-isopropylphenylacetic acid to obtain the desired material; LCMS (M+1) m/z 437.33.

### Example 136

### 8-(4-Ethyl-piperazin-1-yl)-5-methoxy-3,4-dihydro-1H-isoquinoline-2-carboxylic acid (4-propylsulfamoyl-phenyl)-amide

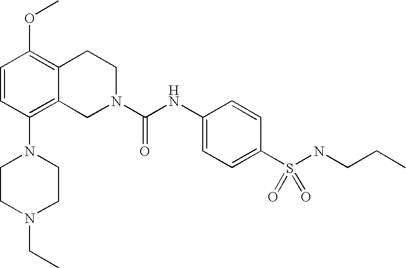

### Example 136a

### 8-(4-Ethyl-piperazin-1-yl)-5-methoxy-isoquinoline

To 30 mL of toluene were added Pd₂(dba)₃ (0.195 g, 0.21 mmole), BINAP (0.293 g, 0.47 mmole), sodium tert-butoxide (1.21 g, 12.6 mmole), 8-Bromo-5-methoxy-isoquinoline (Example 1b) (2.38 g, 10 mmole) and the reaction mixture was evacuated under nitrogen three times. Upon adding N-ethylpiperizine (1.54 g, 13.5 mmole) the reaction mixture was heated to reflux for 16 h. At the end of this period the reaction mixture was cooled to room temperature, diluted with ethyl acetate and washed with sodium carbonate solution. Upon drying over potassium carbonate and concentration under reduced pressure the product was purified by fcc to obtain the desired 8-(4-Ethyl-piperazin-1-yl)-5-methoxy-isoquinoline (2.16 g); LCMS (M+1) m/z 272.23.

### Example 136b

### 8-(4-Ethyl-piperazin-1-yl)-5-methoxy-1,2,3,4-tetrahydro-isoquinoline

A solution of 8-(4-Ethyl-piperazin-1-yl)-5-methoxy-isoquinoline Example 136a (2.16 g, 8 mmole) in 50 mL of acetic acid was treated with Pt(IV)O (35 mg) and hydrogenated at 40 psi for 16 h. At the end of this period the reaction mixture was filtered through diatomaceous earth, concentrated under reduced pressure, diluted with dichloromethane and washed with sodium carbonate solution. Upon drying over potassium carbonate and concentration under reduced pressure the organic layer afforded the crude product which was purified by fcc to obtain the desired 8-(4-Ethyl-piperazin-1-yl)-5-methoxy-1,2,3,4-tetrahydroisoquinoline. (0.717 g); LCMS (M+1) m/z 276.

### Example 136

### 8-(4-Ethyl-piperazin-1-yl)-5-methoxy-3,4-dihydro-1H-isoquinoline-2-carboxylic acid (4-propylsulfamoyl-phenyl)-amide

A method similar to the one described for Example 85 was used except that 8-(4-Ethyl-piperazin-1-yl)-5-methoxy-1,2,3,4-tetrahydro-isoquinoline (Example 136b) was used in place of corresponding N-methylpiperizenyltetrahydroisoquinoline, 4-(n-propylsulfonamido)-phenylacetic acid was used instead of 4-isopropylphenylacetic acid and the reaction was carried out on 1 mmole scale to obtain the desired material. (0.295 g); LCMS (M+1) m/z 515.35.

### Example 137

### 8-(4-Cyclohexyl-piperazin-1-yl)-5-methoxy-3,4-dihydro-1H-isoquinoline-2-carboxylic acid (4-propylsulfamoyl-phenyl)-amide

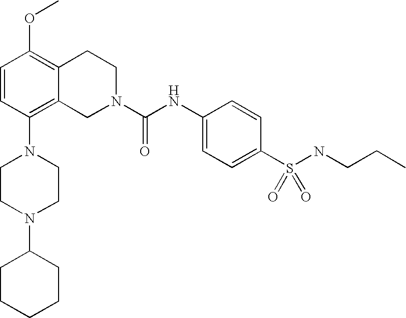

### Example 137a

### 5-Methoxy-8-(4-phenyl-piperazin-1-yl)-isoquinoline

A method similar to the one used for 8-(4-Ethyl-piperazin-1-yl)-5-methoxy-isoquinoline (Example 136) was used except that N-phenylpiperizine was used instead of N-ethylpiperizine to obtain the desired 5-Methoxy-8-(4-phenyl-piperazin-1-yl)-isoquinoline.

LCMS(M+1) m/z 320.

### Example 137b

### 8-N-cyclohexyllpiperazin-1-yltetrahydroisoquinolineisoquinoline

A method similar to the one used for 8-(4-Ethyl-piperazin-1-yl)-5-methoxy-1,2,3,4-tetrahydro-isoquinoline (Example 136) was used except that 5-Methoxy-8-(4-phenyl-piperazin-1-yl)-isoquinoline (Example 137a) was used instead of 8-(4-Ethyl-piperazin-1-yl)-5-methoxy-isoquinoline to obtain the desired 8-N-cyclohexyllpiperazinyltetrahydroisoquinolineisoquinoline. LCMS(M+1) m/z 330.41.

### Example 137

### 8-(4-Cyclohexyl-piperazin-1-yl)-5-methoxy-3,4-dihydro-1H-isoquinoline-2-carboxylic acid (4-propylsulfamoyl-phenyl)-amide

A method similar to the one described for Example 85 was used except that 8-(4-Cyclohexyl-piperazin-1-yl)-5-methoxy-1,2,3,4-tetrahydro-isoquinoline (Example 137b) was used in place of N-methylpiperizenyltetrahydroisoquinoline, 4-(n-propylsulfonamido)phenylacetic acid was used instead of 4-isopropylphenylacetic acid and the reaction was carried out on 1 mmole scale to obtain the desired material. (0.295 g); LCMS (M+1) m/z 569.31.

### Example 138

### 2-(4-Isopropyl-phenyl)-1-(5-methoxy-8-piperazin-1-yl-3,4-dihydro-1H-isoquinolin-2-yl)-ethanone

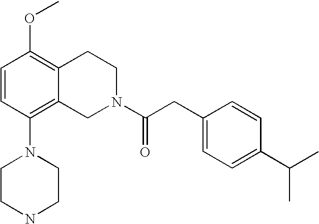

### Example 138a

### 8-N-benzhydryllpipeperizenylisoquinoline

A method similar to the one used for 8-(4-Ethyl-piperazin-1-yl)-5-methoxy-isoquinoline under Example 136 was used except that N-benzhydrylpiperizine was used instead of N-ethylpiperizine to obtain the desired 8-N-benzhydryllpipeperizenylisoquinoline. LCMS(M+1) m/z 410.29.

### Example 138b

### 1-[8-(4-Benzhydryl-piperazin-1-yl)-5-methoxy-3,4-dihydro-1H-isoquinolin-2-yl]-2′-(4-isopropyl-phenyl)-ethanone

A method similar to the one used for 8-(4-Ethyl-piperazin-1-yl)-5-methoxy-1,2,3,4-tetrahydro-isoquinoline (Example 136) was used except that N-benzhydrilpiperizinylisoquinoline (Example 138a) was used instead of 8-(4-ethyl-piperazin-1-yl)-5-methoxy-isoquinoline to obtain the desired 8-N-benzhydrylpipeperizenyltetrahydroisoquinolineisoquinoline. LCMS(M+1) m/z 414.3.

### Example 138c

### 1-[8-(4-Benzhydryl-piperazin-1-yl)-5-methoxy-3,4-dihydro-1H-isoquinolin-2-yl]-2-(4-isopropyl-phenyl)-ethanone

A method similar to the one described for Example 85 was used except that 8-N-benzhydrylpipeperizenyltetrahydroisoquinolineisoquinoline 5 was used instead of 8-N-methylpiperizinyltetrahydroisoquinoline and the reaction was carried out in 1 mmole scale to obtain the desired material 1-[8-(4-Benzhydryl-piperazin-1-yl)-5-methoxy-3,4-dihydro-1H-isoquinolin-2-yl]-2-(4-isopropyl-phenyl)-ethanone. (0.51 g).

### Example 138

### 2-(4-Isopropyl-phenyl)-1-(5-methoxy-8-piperazin-1-yl-3,4-dihydro-1H-isoquinolin-2-yl)-ethanone

A solution of 1-[8-(4-Benzhydryl-piperazin-1-yl)-5-methoxy-3,4-dihydro-1H-isoquinolin-2-yl]-2-(4-isopropyl-phenyl)-ethanone (Example 138b) (0.510 g, 0.89 mmole) in 10 mL of trethylsilane was treated with 10 mL of trifluoroacetic acid and refluxed for 4 h. At the end of this period the reaction mixture was concentrated under reduced pressure, dissolved in 20 mL of ether and extracted with 10 mL of water. The aqueous layer was diluted with 10 mL of acetonitrile and purified by prep HPLC to afford the desired product. (0.4 g); LCMS (M+1) m/z 408.38.

### Example 139

### H-(4-{2-[5-Methoxy-8-(4-methyl-piperazin-1-yl)-3,4-dihydro-1H-isoquinolin-2-yl]-2-oxo-ethyl}-phenyl)-2-phenyl-acetamide

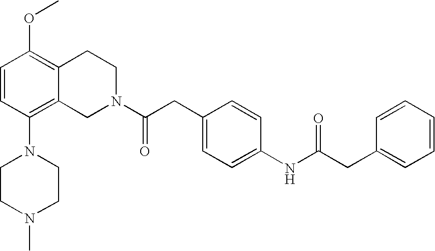

### Example 139a

### 1-[5-Methoxy-8-(4-methyl-piperazin-1-yl)-3,4-dihydro-1H-isoquinolin-2-yl]-2-(4-nitro-phenyl)-ethanone

A solution of 4-nitrophenylacetic acid (2.174 g, 12 mmole) in 100 mL of dichloromethane was cooled to 0 C and treated with a drop of dimethylformamide followed by oxalylchloride (1.74 mL, 20 mmole). The reaction mixture was allowed to warm to room temperature and stirred for 4 h and at the end of this period it was concentrated under reduced pressure. The residue was dissolved in 100 mL of dichloromethane, cooled to 0 C and treated with trethylamine (2.8 mL, 20 mmole) followed by 5-Methoxy-8-(4-methyl-piperazin-1-yl)-1,2,3,4-tetrahydro-isoquinoline (Example 1d) (2.61 g, 10 mmole) and stirred at room temperature for 16 h. At the end of this period the reaction mixture was diluted with dichloromethane, washed with a solution of sodium carbonate, dried over potassium carbonate and concentrated under reduced pressure. The crude product thus obtained was chromatographer over silica gel to afford the desired 1-[5-Methoxy-8-(4-methyl-piperazin-1-yl)-3,4-dihydro-1H-isoquinolin-2-yl]-2-(4-nitro-phenyl)-ethanone. (3.14 g): LCMS (M+1) m/z 425.32.

### Example 139b

### 2-(4-Amino-phenyl)-1-[5-methoxy-8-(4-methyl-piperazin-1-yl)-3,4-dihydro-1H-isoquinolin-2-yl]-ethanone

The amide Example 139a was dissolved in 100 mL methanol and treated with 1 mL of hydrochloric acid followed by 10% Pd/C (50 mg) and hydrogenated at 40 PSI for 16 h. Filtration through diatomaceous earth and concentration under reduced pressure afforded the desired amine 2-(4-Amino-phenyl)-1-[5-methoxy-8-(4-methyl-piperazin-1-yl)-3,4-dihydro-1H-isoquinolin-2-yl]-ethanone. LCMS(M+1) m/z 395.31.

### Example 139

### H-(4-{2-[5-Methoxy-8-(4-methyl-piperazin-1-yl)-3,4-dihydro-1H-isoquinolin-2-yl]-2-oxo-ethyl}-phenyl)-2-phenyl-acetamide

A solution of phenylacetylchloride (0.093 g, 1.2 mmole) in 10 mL of dichloromethane was treated with 2-(4-Amino-phenyl)-1-[5-methoxy-8-(4-methyl-piperazin-1-yl)-3,4-dihydro-1H-isoquinolin-2-yl]-ethanone (0.394 g, 1 mmole) (Example 139b) followed by triethylamine (0.42 mL, 3 mmole). The reaction mixture was stirred for 16 h and upon treatment with methanol was purified by fcc to afford the desired product N-(4-{2-[5-Methoxy-8-(4-methyl-piperazin-1-yl)-3,4-dihydro-1H-isoquinolin-2-yl]-2-oxo-ethyl}-phenyl)-2-phenyl-acetamide. (88 mg); LCMS 513.4.

### Example 140

### N-(4-{2-[5-Methoxy-8-(4-methyl-piperazin-1-yl)-3,4-dihydro-1H-isoquinolin-2-yl]-2-oxo-ethyl}-phenyl)-3-phenyl-propionamide

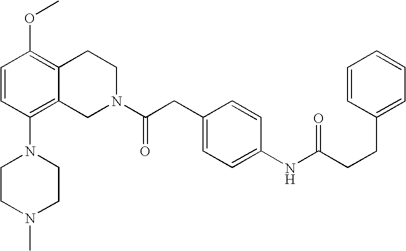

### Example 140

### N-(4-{2-[5-Methoxy-8-(4-methyl-piperazin-1-yl)-3,4-dihydro-1H-isoquinolin-2-yl]-2-oxo-ethyl}-phenyl)-3-phenyl-propionamide

A method similar to the one described for Example 139 was used except that hydrocinnamoylchloride was used instead of phenacetylchloride to obtain the desired material H-(4-{2-[5-Methoxy-8-(4-methyl-piperazin-1-yl)-3,4-dihydro-1H-isoquinolin-2-yl]-2-oxo-ethyl}-phenyl)-3-phenyl-propionamide 3 (164 mg); LCMS (M+1) m/z 527.25.

### Example 141

### N-(4-{2-[5-Methoxy-8-(4-methyl-piperazin-1-yl)-3,4-dihydro-1H-isoquinolin-2-yl]-2-oxo-ethyl}-phenyl)-benzamide

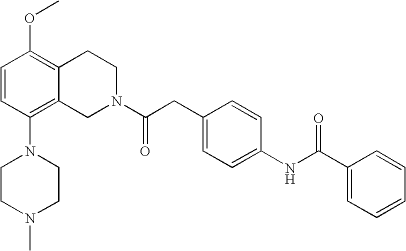

### Example 141

### N-(4-{2-[5-Methoxy-8-(4-methyl-piperazin-1-yl)-3,4-dihydro-1H-isoquinolin-2-yl]-2-oxo-ethyl}-phenyl)-benzamide

A method similar to the one described for Example 139 was used except that benzoylchloride was used instead of phenacetylchloride to obtain the desired material N-(4-{2-[5-Methoxy-8-(4-methyl-piperazin-1-yl)-3,4-dihydro-1H-isoquinolin-2-yl]-2-oxo-ethyl}-phenyl)-benzamide (84 mg); LCMS (M+1) m/z 499.24.

### Example 142

### N-(4-{2-[5-Methoxy-8-(4-methyl-piperazin-1-yl)-3,4-dihydro-1H-isoquinolin-2-yl]-2-oxo-ethyl}-phenyl)-benzenesulfonamide

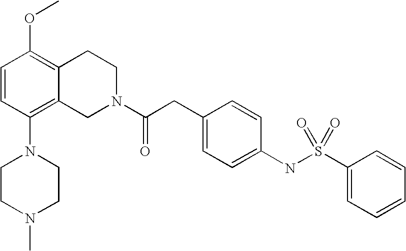

### Example 142

### N-(4-{2-[5-Methoxy-8-(4-methyl-piperazin-1-yl)-3,4-dihydro-1H-isoquinolin-2-yl]-2-oxo-ethyl}-phenyl)-benzenesulfonamide

A method similar to the one described for Example 139 was used except that benzesulfonyl chloride was used instead of phenacetylchloride to obtain the desired material N-(4-{2-[5-Methoxy-8-(4-methyl-piperazin-1-yl)-3,4-dihydro-1H-isoquinolin-2-yl]-2-oxo-ethyl}-phenyl)-benzenesulfonamide (100 mg), LCMS (M+1) m/z 535.26.

### Example 143

### 1-[5-Methoxy-8-(4-methyl-piperazin-1-yl)-3,4-dihydro-1H-isoquinolin-2-yl]-2-(4-phenylmethanesulfonylmethyl-phenyl)-ethanone

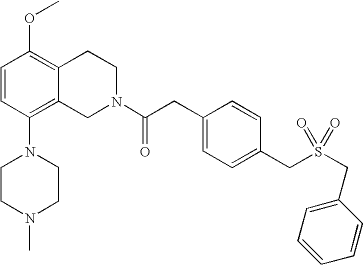

### Example 143

### 1-[5-Methoxy-8-(4-methyl-piperazin-1-yl)-3,4-dihydro-1H-isoquinolin-2-yl]-2-(4-phenylmethanesulfonyl-methyl-phenyl)-ethanone

A method similar to the one described for Example 139 was used except that alpha-toluenesulfonyl chloride was used instead of phenacetylchloride to obtain the desired material 1-[5-Methoxy-8-(4-methyl-piperazin-1-yl)-3,4-dihydro-1H-isoquinolin-2-yl]-2-(4-phenylmethanesulfonylmethyl-phenyl)-ethanone (40 mg); LCMS (M+1) m/z 549.27.

### Example 144

### 4-Chloro-N-(4-{2-[5-methoxy-8-(4-methyl-piperazin-1-yl)-3,4-dihydro-1H-isoquinolin-2-yl]-2-oxo-ethyl}-phenyl)-benzenesulfonamide

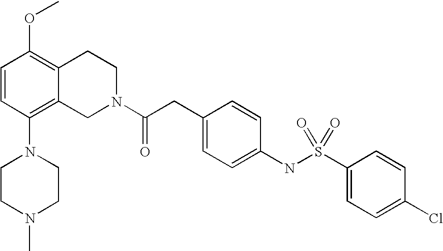

### Example 144

### 4-Chloro-N-(4-{2-[5-methoxy-8-(4-methyl-piperazin-1-yl)-3,4-dihydro-1H-isoquinolin-2-yl]-2-oxo-ethyl}-phenyl)-benzenesulfonamide

A method similar to the one described for Example 139 was used except that p-chloro-benzenesulfonyl chloride was used instead of phenacetyl chloride to obtain the desired material 4-Chloro-N-(4-{2-[5-methoxy-8-(4-methyl-piperazin-1-yl)-3,4-dihydro-1H-isoquinolin-2-yl]-2-oxo-ethyl}-phenyl)-benzenesulfonamide (70 mg); LCMS (M+1) m/z 568.99.

### Example 145

### 4-tert-Butyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide

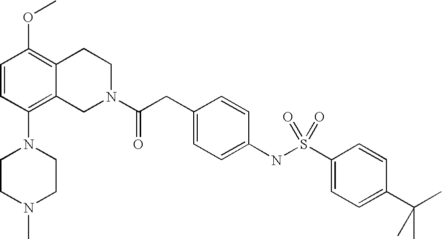

### Example 145

### 4-tert-Butyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide

A method similar to the one described for Example 139 was used except that p-tert-butylbenzenesulfonyl chloride was used instead of phenacetyl chloride to obtain the desired material 4-tert-Butyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide (130 mg); LCMS (M+1) m/Z 591.12.

### Example 146

### H-Benzyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide

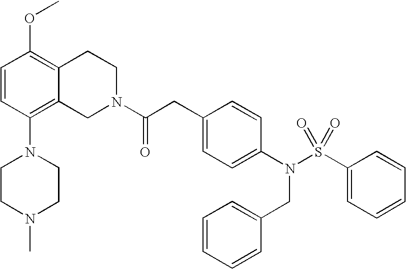

### Example 146a

### 2-(4-Benzylamino-phenyl)-1-[5-methoxy-8-(4-methyl-piperazin-1-yl)-3,4-dihydro-1H-isoquinolin-2-yl]-ethanone

A solution of 2-(4-Amino-phenyl)-1-[5-methoxy-8-(4-methyl-piperazin-1-yl)-3,4-dihydro-1H-isoquinolin-2-yl]-ethanone (0.431 g, 1 mmole) in 10 mL of methanol was treated with triethylamine (0.28 mL, 2 mmole) followed by benzaldehyde (0.106 g, 1 mmole) and acetic acid (0.24 mL, 4 mmole). The resulting reaction mixture was treated with sodium cyanoborohydride (100 mg) and stirred for 16 h. Upon concentration under reduced pressure the reaction product was dissolved in dichloromethane and washed with sodium carbonate solution. The organic layer was dried over potassium carbonate and concentrated under reduced pressure to afford the desired material. LCMS (M+1) m/z 485.37.

### Example 146

### H-Benzyl-N-(4-{2-[5-methoxy-8-(4-methyl-piperazin-1-yl)-3,4-dihydro-1H-isoquinolin-2-yl]-2-oxo-ethyl}-phenyl)-benzenesulfonamide

A method similar to the one described for Example 139 was used except that benzenesulfonyl chloride was used instead of phenacetyl chloride and 2-(4-Benzylamino-phenyl)-1-[5-methoxy-8-(4-methyl-piperazin-1-yl)-3,4-dihydro-1H-isoquinolin-2-yl]-ethanone (Example 146a) was used instead of 2-(4-Amino-phenyl)-1-[5-methoxy-8-(4-methyl-piperazin-1-yl)-3,4-dihydro-1H-isoquinolin-2-yl]-ethanone to obtain the desired material. (130 mg); LCMS (M+1) m/z 591.12.

### Example 147

### 1-(4-{2-[5-Methoxy-8-(4-methyl-piperazin-1-yl)-3,4-dihydro-1H-isoquinolin-2-yl]-2-oxo-ethyl}-phenyl)-3-(4-methoxy-phenyl)-urea

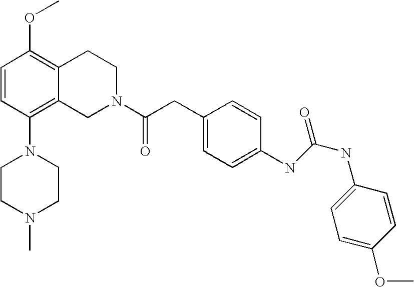

### Example 147

### 1-(4-{2-[5-Methoxy-8-(4-methyl-piperazin-1-yl)-3,4-dihydro-1H-isoquinolin-2-yl]-2-oxo-ethyl}-phenyl)-3-(4-methoxy-phenyl)-urea

To a solution of 2-(4-amino-phenyl)-1-[5-methoxy-8-(4-methyl-piperazin-1-yl)-3,4-dihydro-1H-isoquinolin-2-yl]-ethanone (Example 84) (0.5 mmole, 0.197 g) in 5 mL of acetonitrile was added p-methoxyphenylisocyanate (0.075 g, 0.5 mmole) and triethylamine (0.21 g, 1.5 mmole) and the reaction mixture was stirred for 16 h. At the end of this period the product was purified by prep HPLC to obtain the desired compound. (0.23 g) LCMS (M+1) m/z 544.33.

### Example 148

### 1-(4-{2-[5-Methoxy-8-(4-methyl-piperazin-1-yl)-3,4-dihydro-1H-isoquinolin-2-yl]-2-oxo-ethyl}-phenyl)-3-(3-methoxy-phenyl)-urea

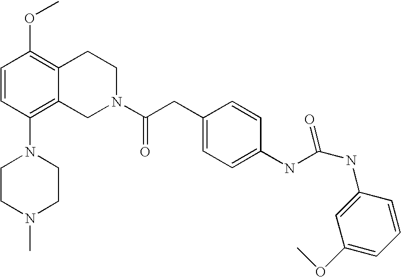

### Example 148

### 1-(4-{2-[5-Methoxy-8-(4-methyl-piperazin-1-yl)-3,4-dihydro-1H-isoquinolin-2-yl]-2-oxo-ethyl}-phenyl)-3-(3-methoxy-phenyl)-urea

A method similar to the one described for Example 147 was used except that 3-methoxyphenyisocyanate was used instead of 4-methoxyisocyanate to afford the desired, product (253 mg). LCMS (M+1) m/z 544.36.

### Example 149

### [5-Methoxy-8-(4-methyl-piperazin-1-yl)-3,4-dihydro-1H-isoquinolin-2-yl]-[2′-methyl-4′-(5-methyl-[1,2,4]oxadiazol-3-yl)-biphenyl-4-yl]-methanone

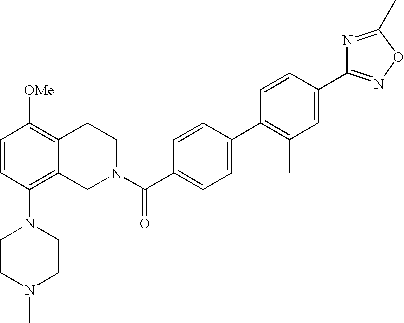

### Example 149a

### 2′-Methyl-4′-(5-methyl-[1,2,4]oxadiazol-3-yl)-biphenyl-4-carboxylic acid

This compound was prepared as described by Oxford, A. W.; Mitchell, W. L.; Bradshaw, J.; Clitherow, J. W.; Carter, M. Azotylpiperazine benzamide derivatives as 5-HT_{1D} antagonists. Eur. Pat. Appl 0 533 268 A1, March 1993; Chem. Abstr. 1993, 119, 1178270e.

### Example 149

### [5-Methoxy-8-(4-methyl-piperazin-1-yl)-3,4-dihydro-1H-isoquinolin-2-yl]-[2′-methyl-4′-(5-methyl-[1,2,4]oxadiazol-3-yl)-biphenyl-4-yl]-methanone

2′-Methyl-4′-(5-methyl-[1,2,4]oxadiazol-3-yl)-biphenyl-4-carboxylic acid (147 mg, 0.50 mmol) was reacted with 5-methoxy-8-(4-methyl-piperizin-1-yl)-1,2,3,4-tetrahydro-isoquinoline (133 mg, 0.51 mmol) using standard HATU coupling conditions as described in example 1. Product was purified by fcc on silica to give 220 mg of an off-white foam. MS: m/z 538 (M+H).

### Example 150

### 5-Methoxy-8-(4-methyl-piperazin-1-yl)-3,4-dihydro-1H-isoquinoline-2-carboxylic acid (4-piperidin-1-yl-phenyl)-amide

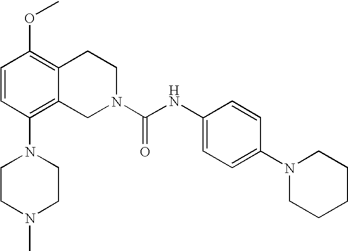

### Example 150a

### 1-(4-Nitro-phenyl)-piperidine

To an ace pressure tube was added 1.0 mL (9.4 mmol) 4-nitrofluorobenzene, 1.4 mL (10 mmol) triethylamine, and 1.0 mL (10 mmol) piperidine. Flask was sealed, heated to 80° C. for 2 h, then cooled to room temperature. Solidified mass was dissolved in 50 mL DCM and extracted with 20% K₂CO₃. Organic layer was dried over Na₂SO₄, filtered, and evaporated. Solid product was pumped down under high vacuum overnight (yielded 1.95 g). MS: m/z 207 (M+H).

### Example 150b

### 4-Piperidin-1-yl-phenylamine

To a 250 mL Parr shaker flask was added 200 mg 5% Pd/C followed by 1-(4-nitro-phenyl)-piperidine (Example 150a) (1.95 g, 9.46 mmol) dissolved in 90 mL EtOH:THF (2:1). Mixture was degassed and backfilled with hydrogen (3 cycles), pressurized to 50 psi hydrogen, and agitated for 5 h. Mixture was filtered through diatomaceous earth (filter cake was washed with EtOH). Filtrate and washings were combined and evaporated. Product was pumped down under high vacuum overnight. Crude product was purified by fcc on silica (eluent—CH₂Cl₂ [symbol] 10:1, CH₂Cl₂:EtOAc [symbol] 5:1, CH₂Cl₂:EtOAc, CH₂Cl₂:EtOAc [symbol] 1:1) to give 0.72 g of an oil. ¹H NMR (CDCl₃) δ 6.87-6.77 (dm), 6.67-6.58 (dm), 3.92 (br s), 3.05-2.94 (m), 1.79-1.63 (m), 1.57-1.47 (m).

### Example 150

### 5-Methoxy-8-(4-methyl-piperazin-1-yl)-3,4-dihydro-1H-isoquinoline-2-carboxylic acid (4-piperidin-1-yl-phenyl)-amide

5-Methoxy-8-(4-methyl-piperizin-1-yl)-1,2,3,4-tetrahydro-isoquinoline (251 mg, 0.96 mmol) was reacted with 4-piperidin-1-yl-phenylamine (Example 150b) (179 mg, 1.02 mmol) and 1,1′-carbonyldiimidazole (160 mg, 0.99 mmol) using a standard method described in example 3. Product was purified by fcc on silica to give 332 mg of an off-white powder. MS: m/z 464 (M+H).

### Example 151

### 5-Methoxy-8-(4-methyl-piperazin-1-yl)-3,4-dihydro-1H-isoquinoline-2-carboxylic acid [4-(4-methyl-piperazin-1-yl)-phenyl]-amide

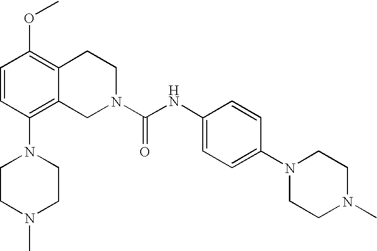

### Example 151a

### 1-Methyl-4-(4-nitro-phenyl)-piperazine

4-Nitrofluorobenzene (1.0 mL, 9.4 mmol) was reacted with N-methylpiperazine (1.1 mL, 9.9 mmol) using a standard method described in example 149. Solid product was pumped down under high vacuum overnight (yielded 2.02 g). MS: m/z 222 (M+H).

### Example 151b

### 4-(4-Methyl-piperazin-1-yl)-phenylamine

1-Methyl-4-(4-nitro-phenyl)-piperazine (Example 151a) was reduced under hydrogen atmosphere in the presence of 5% Pd/C using a standard method described in example 150. Product was purified by fcc on silica to give 0.89 g of a light purple solid. ¹H NMR (CDCl₃) δ 6.86-6.77 (dm), 6.68-6.57 (dm), 3.42 (br s), 3.10-3.04 (m), 2.60-2.54 (m), 2.34 (s).

### Example 151

### 5-Methoxy-8-(4-methyl-piperazin-1-yl)-3,4-dihydro-1H-isoquinoline-2-carboxylic acid [4-(4-methyl-piperazin-1-yl)-phenyl]-amide

5-Methoxy-8-(4-methyl-piperizin-1-yl)-1,2,3,4-tetrahydro-isoquinoline (248 mg, 0.95 mmol) was reacted with 4-(4-methyl-piperazin-1-yl)-phenylamine (Example 151b) (196 mg, 1.02 mmol) and 1,1′-carbonyldiimidazole (154 mg, 0.95 mmol) using a standard method described in example 3. Product was purified by fcc on silica to give 323 mg of an off-white powder. MS: m/z 479 (M+H).

1. A composition of the formula I: 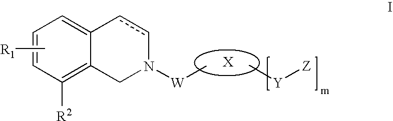
wherein
X is aryl, substituted aryl, heterocyclic or substituted heterocyclic;
W is —(C═O)—, —C(═O)NR^{a}—, —NR^{a}C(═O)—, —C(═O)(CH₂)ₙNR^{a}C(═O)—, —C(═S)NR^{a}—, —C(═O)CH₂O—, —SO₂NR^{a}—, —NR^{a}SO₂—, —CH₂NR^{a}—, —C(═O)CH₂—, —CH₂C(═O)— or 5-membered heterocyclic;
R^{a} is —H, alkyl or substituted alkyl;
n is an integer selected from 0, 1, 2, 3 and 4;
Y is —CH₂—, —O—, —S—, —S(═O)—, —C(═O)—, —SO₂—, —N(R^{b})—, —N(R^{b})SO₂—, —SO₂NR^{b}— or a single bond;
Z is −R^{b}, aryl, substituted aryl, heterocyclic, substituted heterocyclic, aryl(C₁-C₄)alkyl, substituted aryl(C₁-C₄)alkyl, —C(═O)OR^{a}, —C(═O)NR^{a}₂, —NHR^{b}, (R^{a})₂N(C₁-C₆)alkyl or —SO₂R^{c};
R^{b} is —H, alkyl, alkanoyl, (C₁-C₆)alkylsulfanyl, aryl, aryl(C₁-C₄)alkyl or aryl(C₁-C₃)alkoxy(C₁-C₄)alkyl;
R^{c} is alkyl, aryl or heterocyclic;
m is an integer selected from 0 and 1;
R¹ is alkyl, halogen, —OR^{a}, —SOₚR^{a}, —NR^{a}₂ or —CN;
p is an integer selected from 0, 1 and 2;
R² is aryl, heterocyclic or a carboxamide wherein the two substituents of the carboxamide nitrogen form a heterocycle containing said amide nitrogen; and [symbol]
indicates that the bond represented includes single bonds and double bonds.

2. The composition as recited in claim 1 wherein R² is represented by the formula II; 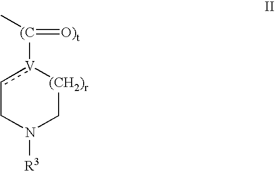
wherein V is N or C;
t is an integer selected from 0 and 1;
r is an integer selected from 1, 2 and 3; [symbol]
indicates that the bond represented includes single bonds and double bonds; and
R³ is —H, alkyl, substituted alkyl, cycloalkyl, substituted cycloalkyl, aryl(C₁-C₄)alkyl or substituted aryl(C₁-C₄)alkyl.

3. The composition as recited in claim 1 wherein [symbol] within formula I represents a single bond.

4. The composition as recited in claim 1 wherein X is phenyl or a 6-membered heterocycle.

5. The composition as recited in claim 1 wherein m is 1.

6. The composition as described in claim 2 wherein;
r is 1;
V is N;
R³ is (C₁-C₄)alkyl or substituted (C₁-C₄)alkyl; and [symbol]
is a single bond.

7. A composition of the formula I: 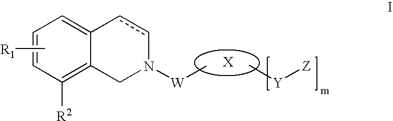
wherein
X is represented by aryl rings, heteroaryl rings or bicyclic heteroaryl ring systems;
W is represented by —(C═O)—, —C(═O)NR^{a}—, —NR^{a}C(═O)—, —C(═O)(CH₂)ₙNR^{a}C(═O)—, —C(═S)NR^{a}—, —C(═O)CH₂O—, —SO₂NR^{a}—, —NR^{a}SO₂—, —CH₂NR^{a}—, —C(═O)CH₂—, —CH₂C(═O)— or 5-membered heteroaryl rings;
R^{a} is represented by H or (C₁-C₆)alkyl;
n is an integer selected from 0, 1, 2, 3 and 4;
Y is represented by —CH₂—, —O—, —S—, —S(═O)—, —C(═O)—, —SO₂—, —N(R^{b})—, —N(R)SO₂—, —SO₂NR^{b}—, or a single bond;
Z is represented by —R^{b}, optionally substituted aryl rings, optionally substituted heteroaryl rings, optionally substituted heterocyclyl rings, optionally substituted aryl(C₁-C₄)alkyl, —C(═O)OR^{a}, —C(═O)NR^{a}₂, NHR^{b}, (R^{a})₂N(C₁-C₆)alkyl or —SO₂R^{c};
R^{b} is represented by H, (C₁-C₆)alkyl, (C₁-C₆)alkanoyl, (C₁-C₆)sulfanyl or aryl rings;
R^{c} is represented by (C₁-C₆)alkyl or aryl rings;
m is an integer selected from 0 and 1;
R¹ is (C₁-C₆)alkyl, (C₃-C₈)cycloalkyl, halogen, —OR^{a}, SOₚR^{a}, NR^{a}₂, or
p is an integer selected from 0, 1 and 2;
R² is aryl or heterocyclic; [symbol]
indicates that the bond represented includes single bonds and double bonds.

8. The composition as recited in claim 7 wherein R² is represented by the formula II; 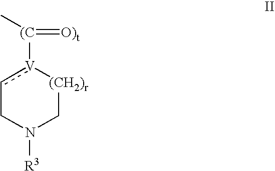
wherein V is N or C;
t is an integer selected from 0 and 1;
r is an integer selected from 1, 2 and 3; [symbol]
indicates that the bond represented includes single bonds and double bonds; and
R³ is —H, (C₁-C₆)alkyl, (C₃-C₆)cycloalkyl or aryl(C₁-C₄)alkyl.

9. The composition as recited in claim 7 wherein X is phenyl or a 6-membered heterocycle.

10. The composition as recited in claim 7 wherein m is 1.

11. The composition as recited in claim 9 wherein W and Y are para to one another on ring X.

12. The composition as recited in claim 8 wherein;
r is 1;
V is N;
R³ is optionally substituted (C₁-C₄)alkyl; and [symbol]
is a single bond.

13. A composition of the formula I: 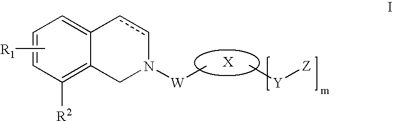
wherein
X is phenyl or pyridyl;
W is —(C═O)—, —C(═O)NR^{a}—, —NR^{a}C(═O)—, —C(═O)(CH₂)ₙNR^{a}C(═O)—, —C(═S)NR^{a}—, —C(═O)CH₂O—, —SO₂NR^{a}—, —NR^{a}SO₂—, —CH₂NR^{a}—, —C(═O)CH₂—, —CH₂C(═O)— or 5-membered heterocyclic;
R^{a} is —H or (C₁-C₆)alkyl;
n is an integer selected from 0, 1, 2, 3 and 4;
Y is —CH₂—, —O—, —S—, —S(═O)—, —C(═O)—, —SO₂—, —N(R^{b})—, —N(R^{b})SO₂—, —SO₂NR^{b}—, or a single bond;
W and Y are oriented para to one-another on ring X;
Z is —R^{b}, aryl, substituted aryl, heterocyclic, substituted heterocyclic, aryl(C₁-C₄)alkyl, substituted aryl(C₁-C₄)alkyl, —C(═O)OR^{a}, —C(═O)NR^{a}₂, —NHR^{b}, (R^{a})₂N(C₁-C₆)alkyl or —SO₂R^{c};
R^{b} is —H, (C₁-C₆)alkyl, (C₁-C₆)alkanoyl, (C₁-C₆)sulfanyl or aryl;
R^{c} is (C₁-C₆)alkyl or aryl;
m is an integer selected from 0 and 1;
R¹ is (C₁-C₆)alkyl, (C₃-C₈)cycloalkyl, halogen, —OR^{a}, —SOₚR^{a}, —NR^{a}₂, or —CN;
p is an integer selected from 0, 1 and 2;
R² is phenyl or a heterocycle containing at least one nitrogen; and [symbol]
indicates that the bond represented includes single bonds and double bonds.

14. The composition as recited in claim 13 wherein R² is represented by the formula II; 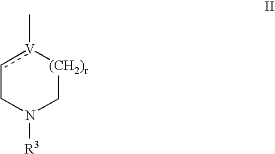
wherein V is represented by N or C;
r is an integer selected from 1, 2 and 3; [symbol]
indicates that the bond represented includes single bonds and double bonds; and
R³ is —H, (C₁-C₄)alkyl, (C₃-C₆)cycloalkyl or aryl(C₁-C₄)alkyl.

15. The composition as recited in claim 13 wherein R¹ is at the 5-position on the isoquinoline ring system;

16. The composition as recited in claim 15 wherein; R¹ is —OR^{a}.

17. The composition as recited in claim 16 wherein;
r is 1;
V is N;
R³ is (C₁-C₄)alkyl; and [symbol]
is a single bond.

18. A compound of any one of claims 1-17 for use in the treatment of depression, generalized anxiety, eating disorders, dementia, panic disorder, sleep disorders, gastrointestinal disorders, motor disorders, endocrine disorders, vasospasm and sexual dysfunction of an animal in need of such therapy.

19. A method of treatment of a human or animal suffering from depression, generalized anxiety, eating disorders, dementia, panic disorder, sleep disorders, gastrointestinal disorders, motor disorders, endocrine disorders, vasospasm and sexual dysfunction administering to such animal an effective amount of a compound of Formula I according to any one of claims 1-17 or a pharmaceutically acceptable salt of said compound.

20. (canceled)
